(19) United States
(12) Patent Application Publication
(10) Pub. No.: US 2011/0136666 A1
(43) Pub. Date: Jun. 9, 2011

(54) CHEMICAL COMPOUNDS

(75) Inventors: William Guy Whittingham, Bracknell (GB); Caroline Louise Winn, Bracknell (GB); John Williams, Bracknell (GB); Harry Glithro, Bracknell (GB)

(73) Assignee: SYNGENTA CROP PROTECTION, INC., Greensboro, NC (US)

(21) Appl. No.: 12/992,270

(22) PCT Filed: Apr. 30, 2009
(86) PCT No.: PCT/GB2009/001100

§ 371 Date: Feb. 24, 2011

(30) Foreign Application Priority Data
May 13, 2008 (GB) 0808664.7

(51) Int. Cl. A01N 25/32; C07D 409/04; A01N 43/54; A01P 13/02; A01P 13/00; C07D 403/04; A01N 43/84; C07D 413/14

(52) U.S. Cl. 504/103; 544/327; 504/239; 544/328; 504/225; 544/122

(57) ABSTRACT

The present invention relates to substituted pyrimidine derivatives as well as N-oxides and agriculturally acceptable salts thereof, and their use to control undesired plant growth, in particular in crops of useful plants. The invention extends to herbicidal compositions comprising such compounds, N-oxides and/or salts as well as mixtures of the same with one or more further active ingredient (such as, for example, an herbicide, fungicide, insecticide and/or plant growth regulator) and/or a safener.

The present invention relates to novel substituted pyrimidine derivatives, as well as N-oxides thereof and agriculturally acceptable salts thereof, and their use to control undesired plant growth, in particular in crops of useful plants. The invention extends to herbicidal compositions comprising such compounds, N-oxides and/or salts as well as mixtures of the same with one or more further active ingredient (such as, for example, an herbicide, fungicide, insecticide and/or plant growth regulator) and/or a safener.

A large number of substituted pyrimidine derivatives are known in the art. For example, WO 2007/083692 discloses aminopyrimidine derivatives wherein inter alia position 2 of the pyrimidine ring is substituted with an optionally substituted imidazole ring. Such compounds are fungicidal and used as plant disease control agents. Additionally, substituted pyrimidine derivatives comprising an optionally substituted cyclopropyl or optionally substituted phenyl group at position 2 in combination with inter alia a nitro or optionally substituted amino group at the position 6 of the pyrimidine ring and their use as herbicides are disclosed in International Patent Publication No. WO 2005/063721. International Patent Publication No. WO 2007/082076 discloses a number of 2-(poly-substituted aryl)-6-amino-5-halo-4-pyrimidine carboxylic acids and their use as herbicides, whilst International Patent Publication No. WO 2007/092184 discloses certain substituted pyrimidine carboxylic acid derivatives as compounds capable of improving the harvestability of crops.

In part, due to the evolution of herbicide-resistant weed populations, and herbicide-resistant crops becoming volunteer weeds, there is a continuing need to control such undesired plant growth in particular in crops of useful plants. Other factors, for example, the demand for cheaper, more effective herbicides, and for herbicides with an improved environmental profile (e.g. safer, less toxic etc.) also drive the need to identify novel herbicidal compounds.

The present invention is based on the finding that certain substituted pyrimidine derivatives, in particular those comprising a 5-6 membered heteroaromatic ring as a substituent at the 2-position of the pyrimidine ring, are particularly good herbicidal compounds.

Thus in a first aspect, the invention provides a method of controlling plant growth (in particular undesired plant growth) which comprises applying to said plants (in particular said undesired plants), a compound of formula (I)

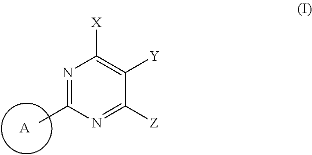

wherein: A is a 5- or 6-membered heteroaromatic ring containing 1-4 heteroatoms and optionally substituted by 1-4 groups R¹, wherein said heteroatom(s) is (are) selected from O, N and S provided said heteroaromatic ring contains only one O or one S atom; each R¹ is independently: halogen, cyano, nitro, azido, hydroxy, alkyl optionally substituted by one or more R^{a}, alkenyl optionally substituted by one or more R^{b}, alkynyl optionally substituted by one or more R^{c}, cycloalkyl optionally substituted by one or more R^{d}, OR^{aa}, S(O)ₐR^{bb}, C(O)R^{cc}, NR^{dd}R^{ee}, SiR^{ff}R^{gg}R^{hh}, P(O)RⁱⁱR^{jj}, or B(OR^{kk})(OR^{LL}); or 2 adjacent R¹ groups together with the atoms to which they are joined form a 5-7 membered ring, said ring optionally containing 1 or 2 heteroatoms selected from O, S and N, and being optionally substituted with 1-4 groups R³; each R³ is independently: halogen; cyano; nitro; hydroxy; alkyl optionally substituted by one or more R^{a}; alkenyl optionally substituted by one or more R^{b}; alkynyl optionally substituted by one or more R^{c}; cycloalkyl optionally substituted by one or more R^{d}; OR^{aa}; S(O)ₐR^{bb}; C(O)R^{cc}; NR^{dd}R^{ee}; SiR^{ff}R^{gg}R^{hh}; (P(O)RⁱⁱR^{jj}; or B(OR^{kk})(OR^{LL}); or any two geminal groups R³ together form a group selected from: oxo; ═CR^{mm}Rⁿⁿ, ═NOR^{oo}, and ═NNR^{pp}R^{qq}; each R^{a} is independently: halogen, cyano, nitro, hydroxy, cycloalkyl, OR^{aa}, S(O)ₐR^{bb}, C(O)R^{cc}, or NR^{dd}R^{ee}; each R^{b} is independently: halogen, cyano, nitro, hydroxy, alkoxy, S(O)₂R^{bb}, C(O)R^{cc}, or P(O)RⁱⁱR^{jj}; each R^{c} is independently: halogen, cyano, alkoxy, S(O)₂R^{bb}, C(O)R^{cc}, or SiR^{ff}R^{gg}R^{hh}; each R^{d} is independently: halogen, cyano, nitro, hydroxy, alkyl, alkenyl, alkynyl, cycloalkyl, alkoxy, S(O)ₐR^{bb}, or C(O)R^{cc}; each R^{aa} is independently: alkyl, haloalkyl, alkoxyalkyl, cycloalkylalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, dialkylaminocarbonyl, alkylimino, or dialkylimino; a is an integer selected from 0, 1 and 2; each R^{bb} is independently: alkyl, haloalkyl, alkoxyalkyl, alkenyl, haloalkenyl, alkynyl, haloalkynyl, cycloalkyl, or alkylcarbonylamino; R^{cc} is: hydrogen, hydroxy, alkyl, haloalkyl, alkoxyalkyl, alkenyl, haloalkenyl, alkynyl, haloalkynyl, cycloalkyl, alkoxy, haloalkoxy, alkoxyalkoxy, arylalkoxy, cycloalkoxy, amino, alkylamino, dialkylamino, or alkylsulphonylamino; R^{dd} is: hydrogen, alkyl, haloalkyl, alkoxyalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl; R^{ee} is: hydrogen, alkyl, haloalkyl, alkoxyalkyl, alkenyl, haloalkenyl, alkynyl, haloalkynyl, or cycloalkyl; or R^{dd} and R^{ee}, together with the N atom to which they are joined form a 4-6 membered ring, optionally containing one further heteroatom selected from O, N and S, said ring being optionally substituted by 1-4 groups selected from: halogen, alkyl, alkoxy, alkylcarbonyl, alkoxycarbonyl, and oxo; R^{ff}, R^{gg}, and R^{hh} are each independently alkyl or haloalkyl; Rⁱⁱ is alkyl, haloalkyl, alkoxy, or haloalkoxy; R^{jj} is alkoxy or haloalkoxy; R^{kk} and R^{LL} are each independently hydrogen or alkyl; or R^{kk} and R^{LL} together with the O atoms and B atom to which they are joined form a 5- or 6-membered heterocyclic ring optionally substituted by 1-4 alkyl groups; R^{mm} is: hydrogen, halogen, cyano, nitro, alkyl, haloalkyl, alkoxyalkyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl; Rⁿⁿ is: hydrogen, halogen, cyano, nitro, alkyl, haloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl or dialkylaminocarbonyl; R^{oo} is: hydrogen, alkyl, haloalkyl, alkoxyalkyl, cycloalkyl, alkylcarbonyl, haloalkylcarbonyl, alkoxycarbonyl, haloalkoxycarbonyl, aminocarbonyl, alkylaminocarbonyl, or dialkylaminocarbonyl; R^{pp} is: hydrogen, alkyl, haloalkyl, alkoxyalkyl, or cycloalkyl; R^{qq} is: hydrogen, alkyl, haloalkyl, alkylcarbonyl, haloalkylcarbonyl, alkoxycarbonyl, aminocarbonyl, alkylaminocarbonyl, or dialkylaminocarbonyl; or R^{pp} and R^{qq} together with the nitrogen to which they are attached form a 5- or 6-membered optionally substituted ring, said ring optionally containing a further heteroatom selected from O, S and N, said substitution(s) being selected from halogen and alkyl; X is nitro, azido, halogen, optionally substituted alkoxy, optionally substituted aminoxy, or NR⁵R⁶, wherein: R⁵ is: hydrogen, optionally substituted alkyl provided said substitution does not comprise a ring system, formyl, optionally substituted alkylcarbonyl provided said substitution does not comprise an aryl moiety, optionally substituted cycloalkylcarbonyl, optionally substituted alkenylcarbonyl, optionally substituted alkynylcarbonyl, optionally substituted phenylcarbonyl, optionally substituted heteroarylcarbonyl, optionally substituted aminocarbonyl, optionally substituted alkoxycarbonyl, optionally substituted alkylsulphonyl, optionally substituted cycloalkylsulphonyl, optionally substituted alkenylsulphonyl, optionally substituted alkynylsulphonyl, optionally substituted phenylsulphonyl, optionally substituted amino, hydroxy, optionally substituted alkoxy, optionally substituted cycloalkoxy, optionally substituted alkylcarbonyloxy, optionally substituted alkoxycarbonyloxy, optionally substituted aminocarbonyloxy, optionally substituted phenoxy, optionally substituted alkenyl provided said substitution does not comprise a ring system, optionally substituted alkynyl provided said substitution does not comprise a ring system, N═CR^{g}R^{h}, cyano, optionally substituted silyl, or optionally substituted phosphonyl; and R⁶ is: hydrogen, optionally substituted alkyl provided said substitution does not comprise a ring system, optionally substituted alkenyl provided said substitution does not comprise a ring system, or optionally substituted alkynyl provided said substitution does not comprise a ring system; or R⁵ and R⁶ together form a group ═C(Rⁱ)OR^{j}, ═C(R^{k})SR^{L}, ═C(R^{m})NRⁿR^{o}; or R⁵ and R⁶ together with the N atom to which they are attached form a 3-8 membered optionally substituted ring system, said ring system optionally containing 1-3 further heteroatoms independently selected from O, S and N; R^{g} is: hydrogen, optionally substituted alkyl, optionally substituted cycloalkyl, optionally substituted phenyl, optionally substituted heteroaryl, optionally substituted amino, optionally substituted alkoxy, optionally substituted phenoxy, optionally substituted alkylthio, or optionally substituted phenylthio; R^{h} is: optionally substituted alkyl, optionally substituted cycloalkyl, optionally substituted phenyl, optionally substituted heteroaryl, optionally substituted amino, optionally substituted alkoxy, or optionally substituted alkylthio; or R^{g} and R^{h} together with the C atom to which they are joined form an optionally substituted 5-7 membered ring system; Rⁱ is hydrogen, optionally substituted alkyl provided said substitution does not comprise a ring system, optionally substituted amino, optionally substituted alkoxy, or optionally substituted alkylthio; R^{j} is optionally substituted alkyl or optionally substituted cycloalkyl; R^{k} is hydrogen, optionally substituted alkyl provided said substitution does not comprise a ring system, optionally substituted amino, or optionally substituted alkylthio; R^{L} is optionally substituted alkyl, or optionally substituted cycloalkyl; R^{m} is hydrogen, optionally substituted alkyl, optionally substituted cycloalkyl, optionally substituted phenyl, optionally substituted heteroaryl, or optionally substituted amino; Rⁿ and R^{o} are each independently hydrogen, optionally substituted alkyl or optionally substituted cycloalkyl; Y is: halogen, cyano, optionally substituted alkyl, optionally substituted cycloalkyl, optionally substituted alkenyl, optionally substituted alkynyl, optionally substituted phenyl, optionally substituted heteroaryl, optionally substituted alkoxy, optionally substituted alkylthio, optionally substituted alkylsulphinyl, optionally substituted alkylsulphonyl, optionally substituted dialkyl phosphonyl, or optionally substituted trialkylsilyl; Z is: (T)ₘ-(U)ₙ—V; wherein, m is an integer of 0 or 1; n is an integer selected from 0, 1, 2 and 3, and n≧m; T is an oxygen or sulphur atom; U is CR^{w}R^{x}; each R^{w} is independently hydrogen, halogen, hydroxy, optionally substituted alkyl, optionally substituted alkoxycarbonyl, or OR^{y} wherein each R^{y} is independently an optionally substituted alkyl or an optionally substituted alkylcarbonyl; each R^{x} is independently hydrogen, halogen, optionally substituted alkyl, or OR^{Z} wherein each R^{Z} is independently an optionally substituted alkyl; or any geminal R^{w} and R^{x} together form a group selected from oxo, or ═NOR^{ca}, wherein R^{ca} is hydrogen or optionally substituted alkyl; or any geminal, vicinal or non-adjacent R^{w} and/or R^{x} together with the C atom(s) to which they are attached and any intervening atom form an optionally substituted 3-6 membered ring; or wherein when at least one R^{w} is OR^{y} and at least one R^{x} is OR^{Z}, said OR^{y} and OR^{Z} groups together with the C atom(s) to which they are attached and any intervening atom form an optionally substituted 5-6 membered heterocyclic ring; V is C(O)R^{cb}, C(S)R^{cd}, C(═NR^{ce})R^{cf}, CHR^{cg}R^{ch}, CH(S[O]ₚR^{ci})(S[O]_{q}R^{cj}), CR^{ck}R^{cl}R^{cm}, or CH₂OR^{cn}; R^{cb} is hydrogen, hydroxy, optionally substituted alkoxy, optionally substituted cycloalkoxy, optionally substituted alkylthio, or optionally substituted amino; R^{cd} is optionally substituted alkoxy, optionally substituted cycloalkoxy, optionally substituted alkylthio, or optionally substituted amino; R^{ce} is hydrogen, optionally substituted alkyl, optionally substituted alkoxy, optionally substituted cycloalkoxy, or optionally substituted amino; R^{cd} is hydrogen, optionally substituted alkoxy, optionally substituted cycloalkoxy, optionally substituted alkylthio, or optionally substituted amino; R^{cg} and R^{ch} are each independently an optionally substituted alkoxy group; or R^{cg} and R^{ch}, together with the carbon to which they are joined, form a dioxolane or dioxane ring, which ring is optionally substituted; R^{ci} and R^{cj} are each independently an optionally substituted alkyl group; or R^{ci} and R^{cj}, together with the carbon and sulphur atoms to which they are joined, form an optionally substituted 5-6 membered ring; p and q are each independently an integer of 0, 1, or 2; R^{ck}, R^{ci}, and R^{cm} are each independently an optionally substituted alkoxy group; or R^{ck} and R^{ci} and R^{cm} together with the carbon to which they are attached form an optionally substituted trioxabicyclo[2.2.2]octane ring system; and R^{cn} is hydrogen or an optionally substituted alkylcarbonyl group.

The compounds of formula (I) may exist in different geometric or optical isomers or different tautomeric forms. One or more centres of chirality may be present, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers or mixtures of isomers. Centres of tautomerisation may be present. This invention covers all such isomers and tautomers and mixtures thereof in all proportions as well as isotopic forms such as deuterated compounds.

For the avoidance of doubt, the term “compound” as used herein includes all salts and N-oxides of said compound.

Suitable salts include those formed by contact with acids or bases. Suitable acid addition salts include those with an inorganic acid such as hydrochloric, hydrobromic, sulfuric, nitric and phosphoric acids, or an organic carboxylic acid such as oxalic, tartaric, lactic, butyric, toluic, hexanoic and phthalic acids, or sulphonic acids such as methane, benzene and toluene sulphonic acids. Other examples of organic carboxylic acids include haloacids such as trifluoroacetic acid.

Suitable salts also include those formed by strong bases (e.g. metal hydroxides—in particular sodium, potassium or lithium—or quaternary ammonium hydroxide) as well as those formed with amines.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

Each alkyl moiety either alone or as part of a larger group (such as alkoxyalkyl, alkylthio, alkylsulphinyl, alkylsulphonyl, alkylcarbonyl, haloalkylcarbonyl etc.) is a straight or branched chain and is, for example, methyl, ethyl, n-propyl, n-butyl, n-pentyl, n-hexyl, iso-propyl, sec-butyl, iso-butyl, tert-butyl or neo-pentyl. The alkyl groups are suitably C₁ to C₁₀ alkyl groups, but are preferably C₁-C₈, even more preferably C₁-C₆ and most preferably C₁-C₄ alkyl groups.

Ring or chain forming alkylene, alkenylene and alkynylene groups can optionally be further substituted by one or more halogen, C₁₋₃alkyl and/or C₁₋₃ alkoxy group.

When present, the optional substituents on an alkyl moiety (alone or as part of a larger group) include one or more of halogen, nitro, cyano, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkylsilyl(C₁₋₆)alkoxy, C₁₋₆ alkoxycarbonyl(C₁₋₁₀)alkoxy, C₁₋₁₀ haloalkoxy, aryl(C₁₋₄)alkoxy (where the aryl group is optionally substituted), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio (where the aryl group is optionally substituted), C₃₋₇ cycloalkylthio (where the cycloalkyl group is optionally substituted with C₁₋₆ alkyl or halogen), tri(C₁₋₄alkylsilyl(C₁₋₆)alkylthio, arylthio (where the aryl group is optionally substituted), C₁₋₆ alkylsulfonyl, C₁₋₆ haloalkylsulfonyl, C₁₋₆ alkylsulfinyl, C₁₋₆ haloalkylsulfinyl, arylsulfonyl (where the aryl group may be optionally substituted), tri(C₁₋₄)alkylsilyl, aryldi(C₁₋₄)alkylsilyl, (C₁₋₄)alkyldiarylsilyl, triarylsilyl, aryl(C₁₋₄)alkylthio(C₁₋₄alkyl, aryloxy(C₁₋₄)alkyl, formyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)aminocarbonyl, N—(C₁₋₃ alkyl)-N—(C₁₋₃ alkoxy)aminocarbonyl, C₁₋₆ alkylcarbonyloxy, arylcarbonyloxy (where the aryl group is optionally substituted), di(C₁₋₆)alkylaminocarbonyloxy, oximes and oxime-ethers such as ═NOalkyl, ═NOhaloalkyl and ═NOaryl (itself optionally substituted), aryl (itself optionally substituted), heteroaryl (itself optionally substituted), heterocyclyl (itself optionally substituted with C₁₋₆ alkyl or halogen), aryloxy (where the aryl group is optionally substituted), heteroaryloxy, (where the heteroaryl group is optionally substituted), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, C₂₋₆ alkenylcarbonyl, C₂₋₆ alkynylcarbonyl, C₃₋₆ alkenyloxycarbonyl, C₃₋₆ alkynyloxycarbonyl, aryloxycarbonyl (where the aryl group is optionally substituted) and arylcarbonyl (where the aryl group is optionally substituted).

Alkenyl and alkynyl moieties can be in the form of straight or branched chains, and the alkenyl moieties, where appropriate, can be of either the (E)- or (Z)-configuration. Examples are vinyl, allyl and propargyl. Alkenyl and alkynyl moieties can contain one or more double and/or triple bonds in any combination. It is understood, that allenyl and alkynylalkenyl are included in these terms.

When present, the optional substituents on alkenyl or alkynyl include those optional substituents given above for an alkyl moiety.

In the context of this specification acyl is optionally substituted C₁₋₆ alkylcarbonyl (for example acetyl), optionally substituted C₂₋₆ alkenylcarbonyl, optionally substituted C₃₋₆ cycloalkylcarbonyl (for example cyclopropylcarbonyl, optionally substituted C₂₋₆ alkynylcarbonyl, optionally substituted arylcarbonyl (for example benzoyl) or optionally substituted heteroarylcarbonyl.

Halogen is fluorine, chlorine, bromine or iodine.

Haloalkyl groups are alkyl groups which are substituted with one or more of the same or different halogen atoms and are, for example, CF₃, CF₂Cl, CF₂H, CCl₂H, FCH₂, ClCH₂, BrCH₂, CH₃CHF, (CH₃)₂CF, CF₃CH₂ or CHF₂CH₂.

In the context of the present specification ring systems may be saturated, unsaturated, or aromatic, and may also be fused, Spiro or bridging ring systems. The terms “aryl”, “aromatic ring” and “aromatic ring system” as used herein refer to ring systems which may be mono-, bi- or tricyclic. Examples of such rings include phenyl, naphthalenyl, anthracenyl, indenyl or phenanthrenyl. A preferred aryl group is phenyl. In addition, the terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl, pyrimidyl, triazinyl, thienyl, furyl, oxazolyl, isoxazolyl, 2,1,3-benzoxadiazole and thiazolyl.

The terms heterocycle and heterocyclyl refer to a non-aromatic preferably monocyclic or bicyclic ring systems containing up to 10 atoms including one or more (preferably one or two) heteroatoms selected from O, S and N. Examples of such rings include 1,3-dioxolane, oxetane, tetrahydrofuran, morpholine, thiomorpholine and piperazine.

In the case of heteroaromatic or heterocyclic rings containing S as a heteroatom, the S atom may also be in the form of a mono- or di-oxide.

When present, the optional substituents on heterocyclyl include C₁₋₆ alkyl and C₁₋₆ haloalkyl, an oxo-group (allowing one of the carbon atoms in the ring to be in the form of a keto group), as well as those optional substituents given above for an alkyl moiety.

Cycloalkyl includes cyclopropyl, cyclobutyl, cyclopentyl and cyclohexyl. Cycloalkylalkyl is preferentially cyclopropylmethyl. Cycloalkenyl includes cyclopentenyl and cyclohexenyl.

When present, the optional substituents on cycloalkyl or cycloalkenyl include C₁₋₃ alkyl as well as those optional substituents given above for an alkyl moiety.

Carbocyclic rings include aryl, cycloalkyl and cycloalkenyl groups.

When present, the optional substituents on aryl or heteroaryl are selected independently, from halogen, nitro, cyano, C₁₋₆ alkyl, C₁₋₆ haloalkyl, C₁₋₆ alkoxy-(C₁₋₆)alkyl, C₂₋₆ alkenyl, C₂₋₆ haloalkenyl, C₂₋₆ alkynyl, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkyl-silyl(C₁₋₆)alkoxy, C₁₋₆ alkoxycarbonyl(C₁₋₁₀)alkoxy, C₁₋₁₀ haloalkoxy, aryl(C₁₋₄)alkoxy (where the aryl group is optionally substituted with halogen or C₁₋₆ alkyl), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio, C₃₋₇ cycloalkylthio (where the cycloalkyl group is optionally substituted with C₁₋₆ alkyl or halogen), tri(C₁₋₄)-alkylsilyl(C₁₋₆)alkylthio, arylthio, C₁₋₆ alkylsulfonyl, C₁₋₆ haloalkylsulfonyl, C₁₋₆ alkylsulfinyl, C₁₋₆ haloalkylsulfinyl, arylsulfonyl, C₁₋₄ alkylsulphonyloxy, tri(C₁₋₄)alkylsilyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)aminocarbonyl, N—(C₁₋₃ alkyl)-N—(C₁₋₃ alkoxy)aminocarbonyl, C₁₋₆ alkylcarbonyloxy, arylcarbonyloxy, di(C₁₋₆)alkylamino-carbonyloxy, aryl (itself optionally substituted with C₁₋₆ alkyl or halogen), heteroaryl (itself optionally substituted with C₁₋₆ alkyl or halogen), heterocyclyl (itself optionally substituted with C₁₋₆ alkyl or halogen), aryloxy (where the aryl group is optionally substituted with C₁₋₆ alkyl or halogen), heteroaryloxy (where the heteroaryl group is optionally substituted with C₁₋₆ alkyl or halogen), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, arylcarbonyl, (where the aryl group is itself optionally substituted with halogen or C₁₋₆ alkyl) or two adjacent positions on an aryl or heteroaryl system may be cyclised to form a 5, 6 or 7 membered carbocyclic or heterocyclic ring, itself optionally substituted with halogen or C₁₋₆ alkyl. Further substituents for aryl or heteroaryl include arylcarbonylamino (where the aryl group is substituted by C₁₋₆ alkyl or halogen), C₁₋₆alkoxycarbonylamino, C₁₋₆alkoxycarbonyl-N—(C₁₋₆)alkylamino, aryloxycarbonylamino (where the aryl group is substituted by C₁₋₆ alkyl or halogen), aryloxycarbonyl-N—(C₁₋₆)alkylamino (where the aryl group is substituted by C₁₋₆ alkyl or halogen), arylsulphonylamino (where the aryl group is substituted by C₁₋₆ alkyl or halogen), arylsulphonyl-N—(C₁₋₆)alkylamino (where the aryl group is substituted by C₁₋₆ alkyl or halogen), aryl-N—(C₁₋₆)alkylamino (where the aryl group is substituted by C₁₋₆ alkyl or halogen), arylamino (where the aryl group is substituted by C₁₋₆ alkyl or halogen), heteroarylamino (where the heteroaryl group is substituted by C₁₋₆ alkyl or halogen), heterocyclylamino (where the heterocyclyl group is substituted by C₁₋₆ alkyl or halogen), aminocarbonylamino, C₁₋₆alkylaminocarbonylamino, di(C₁₋₆)alkylaminocarbonylamino, arylaminocarbonylamino where the aryl group is substituted by C₁₋₆ alkyl or halogen), aryl-N—(C₁₋₆)alkylaminocarbonylamino (where the aryl group is substituted by C₁₋₆ alkyl or halogen), C₁₋₆alkylaminocarbonyl-N—(C₁₋₆)alkylamino, di(C₁₋₆)alkylaminocarbonyl-N—(C₁₋₆)alkylamino, arylaminocarbonyl-N—(C₁₋₆)alkylamino (where the aryl group is substituted by C₁₋₆ alkyl or halogen) and aryl-N—(C₁₋₆)alkylaminocarbonyl-N—(C₁₋₆)alkylamino (where the aryl group is substituted by C₁₋₆ alkyl or halogen).

For substituted phenyl moieties, heterocyclyl and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alkynyl, C₃₋₇ cycloalkyl, nitro, cyano, CO₂H, C₁₋₆ alkylcarbonyl, C₁₋₆ alkoxycarbonyl, aryl, heteroaryl, C₁₋₆ alkylamino, di(C₁₋₆ alkyl)amino, C₁₋₆ alkylaminocarbonyl, or di(C₁₋₆ alkyl)aminocarbonyl.

Haloalkenyl groups are alkenyl groups which are substituted with one or more of the same or different halogen atoms.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

In particularly preferred embodiments of the invention, the preferred groups for A, X, Y, and Z, in any combination thereof, are as set out below.

As described above, A is a 5- or 6-membered heteroaromatic ring containing 1-4 heteroatoms and optionally substituted by 1-4 groups R¹, wherein said heteroatom(s) is (are) selected from O, N and S provided said heteroaromatic ring contains only one O or one S atom.

Preferably A is a ring system selected from: thiophene, furan, pyrrole, isoxazole, isothiazole, oxazole, thiazole, imidazole, pyrazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,3,4-oxatriazole, 1,2,3,4-thiatriazole, tetrazole, pyridine, pyridazine, pyrimidine, pyrazine, 1,2,3-triazine, 1,2,4-triazine, 1,3,5-triazine, and 1,2,4,5-tetrazine, wherein said ring system is optionally substituted by 1-3 groups R¹.

More preferably A is a ring system selected from thiophene, furan, pyrrole, isoxazole, isothiazole, oxazole, thiazole, imidazole, pyrazole, 1,2,4-oxadiazole, 1,3,4-oxadiazole, 1,2,4-thiadiazole, 1,3,4-thiadiazole, 1,2,3-triazole, 1,2,4-triazole, pyridine, pyridazine, pyrimidine, pyrazine, 1,2,4-triazine, and 1,3,5-triazine, wherein said ring system is optionally substituted by 1-3 groups R¹.

Most preferably A is a ring selected from: thiophene, thiazole, 1,2,3-triazole, and pyridine, wherein said ring is optionally substituted by 1-3 groups R¹.

Each R¹ is independently: halogen, cyano, nitro, azido, hydroxy, alkyl optionally substituted by one or more R^{a}, alkenyl optionally substituted by one or more R^{b}, alkynyl optionally substituted by one or more R^{c}, cycloalkyl optionally substituted by one or more R^{d}, OR^{aa}, S(O)ₐR^{bb}, C(O)R^{cc}, NR^{dd}R^{ee}, SiR^{ff}R^{gg}R^{hh}, P(O)RⁱⁱR^{jj}, or B(OR^{kk})(OR^{LL}); or 2 adjacent R¹ groups together with the atoms to which they are joined form a 5-7 membered ring, said ring optionally containing 1 or 2 heteroatoms selected from O, S and N, and being optionally substituted with 1-4 groups R³.

Preferably each R¹ is independently: halogen; cyano; nitro; hydroxy; C₁₋₆ alkyl optionally substituted by 1-4 groups R^{a}; C₁₋₆ haloalkyl optionally substituted by 1-4 groups R^{a}; OR^{aa}; S(O)ₐR^{bb}; C(O)R^{cc}; or NR^{dd}R^{ee}; or 2 adjacent groups R¹ together with the atoms to which they are joined form a 5-6 membered ring, optionally containing 1 or 2 heteroatoms selected from O, N and S, said 5-6 membered ring being optionally substituted with 1-4 groups R³.

More preferably each R¹ is independently: halogen, cyano, C₁₋₄ alkyl, C₁₋₄ haloalkyl, C₁₋₄ alkoxy, C₁₋₄ haloalkoxy, amino, C₁₋₄alkylamino, di(C₁₋₂alkyl)amino, C₁₋₄ alkylthio, or C₁₋₄ haloalkylthio; or 2 adjacent groups R¹ together with the atoms to which they are joined form a 6 membered aromatic ring, optionally substituted with 1-2 groups selected from halogen, cyano, C₁₋₄ alkyl, C₁₋₄ haloalkyl, C₁₋₄ alkoxy, C₁₋₄ haloalkoxy, C₁₋₄ alkylthio, and C₁₋₄ haloalkylthio.

Most preferably each R¹ is independently selected from: halogen, cyano, C₁₋₂ alkyl, C₁₋₂ haloalkyl, C₁₋₂ alkoxy, C₁₋₂ haloalkoxy, and di(C₁₋₂alkyl)amino; or 2 adjacent groups R¹ together with the atoms to which they are joined form a 6 membered aromatic ring optionally substituted with 1-2 groups independently selected from: halogen, cyano, C₁₋₂ alkyl, C₁₋₂ haloalkyl, C₁₋₂ alkoxy, and C₁₋₂ haloalkoxy.

Each R³ is independently: halogen; cyano; nitro; hydroxy; alkyl optionally substituted by one or more R^{a}; alkenyl optionally substituted by one or more R^{b}; alkynyl optionally substituted by one or more R^{c}; cycloalkyl optionally substituted by one or more R^{d}; OR^{aa}; S(O)ₐR^{bb}; C(O)R^{cc}; NR^{dd}R^{ee}; SiR^{ff}R^{gg}R^{hh}; P(O)RⁱⁱR^{jj}; B(OR^{kk})(OR^{LL}); or any two geminal groups R³ together form a group selected from: oxo; ═CR^{mm}Rⁿⁿ, ═NOR^{oo}, and ═NNR^{pp}R^{qq}.

Preferably each R³ is independently: halogen; cyano; nitro; hydroxy; C₁₋₆alkyl optionally substituted by 1-4 groups R^{a}; C₁₋₆haloalkyl optionally substituted by 1-4 groups R^{a}; OR^{aa}; S(O)ₐR^{bb}; C(O)R^{cc}; NR^{dd}R^{ee}; or any two geminal groups R³ together form a group selected from: oxo, ═CR^{mm}Rⁿⁿ, ═NOR^{oo}, and ═NNR^{pp}R^{qq};

Each R^{a} is independently: halogen; cyano; nitro; hydroxy; cycloalkyl; OR^{aa}; S(O)ₐR^{bb}; C(O)R^{cc}; or NR^{dd}R^{ee}.

Preferably each R^{a} is independently: cyano, hydroxy, C₃₋₆ cycloalkyl, OR^{aa}, S(O)ₐR^{bb}, C(O)R^{cc}, or NR^{dd}R^{ee}.

Each R^{b} is independently: halogen; cyano; nitro; hydroxy; alkoxy; S(O)₂R^{bb}; C(O)R^{cc}; or P(O)RⁱⁱR^{jj}.

Each R^{c} is independently: halogen; cyano; alkoxy; S(O)₂R^{bb}; C(O)R^{cc}; or SiR^{ff}R^{gg}R^{hh}.

Each R^{d} is independently: halogen; cyano; nitro; hydroxy; alkyl; alkenyl; alkynyl; cycloalkyl; alkoxy; S(O)ₐR^{bb}; or C(O)R^{cc}.

Each R^{aa} is independently: alkyl, haloalkyl, alkoxyalkyl, cycloalkylalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, dialkylaminocarbonyl, alkylimino, or dialkylimino.

Preferably each R^{aa} is independently: C₁₋₆ alkyl, C₁₋₆ haloalkyl, C₂₋₈ alkoxyalkyl, or C₁₋₆ alkylcarbonyl;

a is an integer selected from 0, 1, and 2.

Each R^{bb} is independently alkyl, haloalkyl, alkoxyalkyl, alkenyl, haloalkenyl, alkynyl, haloalkynyl, cycloalkyl, or alkylcarbonylamino. Preferably each R^{bb} is independently: C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino.

Each R^{cc} is independently hydrogen, hydroxy, alkyl, haloalkyl, alkoxyalkyl, alkenyl, haloalkenyl, alkynyl, haloalkynyl, cycloalkyl, alkoxy, haloalkoxy, alkoxyalkoxy, arylalkoxy, cycloalkoxy, amino, alkylamino, dialkylamino, or alkylsulphonylamino.

Preferably each R^{cc} is independently: hydrogen, hydroxy, C₁₋₆ alkyl, C₁₋₆haloalkyl, C₃₋₆ cycloalkyl, C₁₋₆ alkoxy, phenyl(C₁₋₆)alkoxy, C₃₋₆ cycloalkoxy, amino, C₁₋₆ alkylamino, di(C₁₋₄)alkylamino (also referred to herein as C₂₋₈dialkylamino), or C₁₋₆ alkylsulphonylamino.

R^{dd} is hydrogen, alkyl, haloalkyl, alkoxyalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

R^{ee} is hydrogen, alkyl, haloalkyl, alkoxyalkyl, alkenyl, haloalkenyl, alkynyl, haloalkynyl, or cycloalkyl.

Or R^{dd} and R^{ee}, together with the N atom to which they are joined form a 4-6 membered ring, optionally containing one further heteroatom selected from O, N and S, said ring being optionally substituted by 1-4 groups selected from halogen, alkyl, alkoxy, alkylcarbonyl, alkoxycarbonyl, and oxo.

Preferably R^{dd} is hydrogen, C₁₋₆ alkyl, C₁₋₆ alkylcarbonyl, C₂₋₆ alkoxycarbonyl, C₁₋₆ alkylsulphonyl, C₁₋₆ haloalkylsulphonyl, aminocarbonyl, C₁₋₆ alkylaminocarbonyl, or C₂₋₈ dialkylaminocarbonyl (also referred to herein as di(C₁₋₄alkylaminocarbonyl).

Preferably each R^{ee} is independently: hydrogen, C₁₋₆ alkyl, or C₃₋₆ cycloalkyl; or R^{dd} and R^{ee}, together with the N atom to which they are joined form a 5- or 6-membered saturated ring optionally containing one further heteroatom selected from O, N and S, said ring being optionally substituted by 1 or 2 C₁₋₆ alkyl or oxo groups.

R^{ff}, R^{gg} and R^{hh} are each independently alkyl or haloalkyl. Rⁱⁱ is alkyl, haloalkyl, alkoxy, or haloalkoxy. R^{jj} is alkoxy or haloalkoxy. R^{kk} and R^{LL} are each independently hydrogen or alkyl; or R^{kk} and R^{LL} together with the O atoms and B atom to which they are joined form a 5- or 6-membered heterocyclic ring optionally substituted by 1-4 alkyl groups.

R^{mm} is hydrogen, halogen, cyano, nitro, alkyl, haloalkyl, alkoxyalkyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

Preferably each R^{mm} is independently: hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl.

Rⁿⁿ is hydrogen, halogen, cyano, nitro, alkyl, haloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

Preferably each Rⁿⁿ is independently: hydrogen, halogen, cyano, nitro, C₁₋₆ alkyl, C₁₋₆ alkylcarbonyl, C₁₋₆ alkoxycarbonyl, C₁₋₆ alkylsulphonyl, or aminocarbonyl.

R^{oo} is hydrogen, alkyl, haloalkyl, alkoxyalkyl, cycloalkyl, alkylcarbonyl, haloalkylcarbonyl, alkoxycarbonyl, haloalkoxycarbonyl, aminocarbonyl, alkylaminocarbonyl, or dialkylaminocarbonyl. Preferably each R^{oo} is independently hydrogen, C₁₋₆ alkyl, or C₃₋₆ cycloalkyl.

R^{pp} is hydrogen, alkyl, haloalkyl, alkoxyalkyl, or cycloalkyl.

R^{qq} is hydrogen, alkyl, haloalkyl, alkylcarbonyl, haloalkylcarbonyl, alkoxycarbonyl, aminocarbonyl, alkylaminocarbonyl, or dialkylaminocarbonyl, or R^{pp} and R^{qq} together with the nitrogen to which they are attached form a 5- or 6-membered optionally substituted ring, said ring optionally containing a further heteroatom selected from oxygen, sulphur and nitrogen, said substitution(s) being independently selected from halogen and alkyl.

Preferably each R^{pp} is independently hydrogen, C₁₋₆ alkyl, or C₃₋₆ cycloalkyl.

Preferably each R^{qq} is independently hydrogen or C₁₋₆ alkyl.

As defined herein X is nitro, azido, halogen, optionally substituted alkoxy, optionally substituted aminoxy, or NR⁵R⁶. Preferably X is NR⁵R⁶ or halogen. More preferably X is NR⁵R⁶. Most preferably X is amino.

R⁵ is hydrogen, optionally substituted alkyl provided said substitution does not comprise a ring system, formyl, optionally substituted alkylcarbonyl provided said substitution does not comprise an aryl moiety, optionally substituted cycloalkylcarbonyl, optionally substituted alkenylcarbonyl, optionally substituted alkynylcarbonyl, optionally substituted phenylcarbonyl, optionally substituted heteroarylcarbonyl, optionally substituted aminocarbonyl, optionally substituted alkoxycarbonyl, optionally substituted alkylsulphonyl, optionally substituted cycloalkylsulphonyl, optionally substituted alkenylsulphonyl, optionally substituted alkynylsulphonyl, optionally substituted phenylsulphonyl, optionally substituted amino, hydroxy, optionally substituted alkoxy, optionally substituted cycloalkoxy, optionally substituted alkylcarbonyloxy, optionally substituted alkoxycarbonyloxy, optionally substituted aminocarbonyloxy, optionally substituted alkenyl provided said substitution does not comprise a ring system, optionally substituted alkynyl provided said substitution does not comprise a ring system, N═CR^{g}R^{h}, cyano, optionally substituted silyl, or optionally substituted phosphonyl.

R⁶ is hydrogen, optionally substituted alkyl provided said substitution does not comprise a ring system, optionally substituted alkenyl provided said substitution does not comprise a ring system, or optionally substituted alkynyl provided said substitution does not comprise a ring system; or R⁵ and R⁶ together form a group ═C(Rⁱ)OR^{j}, ═C(R^{k})SR^{L}, or ═C(R^{m})NRⁿR^{o}; or R⁵ and R⁶ together with the N atom to which they are attached form a 3-8 membered optionally substituted ring system, said ring system optionally containing 1-3 further heteroatoms independently selected from O, S and N.

Preferably R⁵ is: hydrogen; alkyl optionally substituted by one or more R^{p}; alkenyl optionally substituted by one or more R^{t}; alkynyl optionally substituted by one or more groups R^{u}; OR^{rr}; SO₂R^{ss}; C(O)R^{uu}; NR^{vv}R^{ww}; N═CR^{g}R^{h}; cyano; trialkylsilyl; or dialkyl phosphonyl.

More preferably R⁵ is: hydrogen; C₁₋₆ alkyl optionally substituted by 1-4 groups R^{p}; C₁₋₆ haloalkyl optionally substituted by 1-4 groups R^{p}; SO₂R^{ss}; or C(O)R^{uu}.

Most preferably R⁵ is: hydrogen; C₁₋₄ alkyl optionally substituted by 1-2 groups R^{p}; C₁₋₄ haloalkyl optionally substituted by 1-2 groups R^{p}; SO₂R^{ss}; or C(O)R^{uu}.

In preferred embodiments R⁶ is hydrogen, alkyl optionally substituted by one or more R^{p}, alkenyl optionally substituted by one or more R^{t}, or alkynyl optionally substituted by one or more groups R^{u}; or R⁵ and R⁶ together form a group ═C(Rⁱ)OR^{j}, ═C(R^{k})SR^{L}, ═C(R^{m})NRⁿR^{o}; or R⁵ and R⁶ together with the N atom to which they are attached form a 3-8 membered ring system, said ring system optionally containing 1-3 further heteroatoms independently selected from O, N and S and said ring system being optionally substituted by 1-4 groups R^{v}.

In more preferred embodiments R⁶ is hydrogen; C₁₋₆ alkyl optionally substituted by 1-4 groups R^{p}; or C₁₋₆haloalkyl optionally substituted by 1-4 groups R^{p}; or R⁵ and R⁶ together with the N atom to which they are joined form a 3-8 membered ring system, said ring system optionally containing 1 or 2 further heteroatoms independently selected from O, N and S and being optionally substituted by 1-2 groups R^{v}; or R⁵ and R⁶ together form a group ═C(Rⁱ)OR^{j}, ═C(R^{k})SR^{L}, or ═C(R^{m})NRⁿR^{o}.

In the most preferred embodiments R⁶ is: hydrogen; C₁₋₄ alkyl optionally substituted by 1-2 groups R^{p}; or C₁₋₄ haloalkyl optionally substituted by 1-2 groups R^{p}; or R⁵ and R⁶ together with the N atom to which they are joined form a 4-6 membered ring system, said ring system optionally containing 1 further heteroatom selected from O, S and N and being optionally substituted by 1-2 groups R^{v}; or R⁵ and R⁶ together form a group selected from: ═C(Rⁱ)OR^{j}, ═C(R^{k})SR^{L}, and ═C(R^{m})NRⁿR^{o}.

R^{g} is: hydrogen, optionally substituted alkyl, optionally substituted cycloalkyl, optionally substituted phenyl, optionally substituted heteroaryl, optionally substituted amino, optionally substituted alkoxy, optionally substituted phenoxy, optionally substituted alkylthio, or optionally substituted phenylthio.

R^{h} is: optionally substituted alkyl, optionally substituted cycloalkyl, optionally substituted phenyl, optionally substituted heteroaryl, optionally substituted amino, optionally substituted alkoxy, or optionally substituted alkylthio. Alternatively R^{g} and R^{h} together with the C atom to which they are joined form an optionally substituted 5-7 membered ring system.

Preferably R^{g} and R^{h} are each independently: hydrogen; alkyl optionally substituted by one or more R^{xx}; cycloalkyl substituted by one of more R^{yy}; phenyl substituted by one or more R^{zz}; heteroaryl substituted by one of more R^{ab}; NR^{ac}R^{ad}; OR^{ae}; or SR^{af}; or R^{g} and R^{h} together with the C atom to which they are joined form a 5-7 membered ring system optionally substituted by 1-4 groups R^{ag}.

Rⁱ is hydrogen, optionally substituted alkyl provided said substitution does not comprise a ring system, optionally substituted amino, optionally substituted alkoxy, or optionally substituted alkylthio;

Preferably Rⁱ is: hydrogen; alkyl optionally substituted by one or more R^{xx}; NR^{ac}R^{ad}; OR^{ae}; or SR^{af}. More preferably Rⁱ is: hydrogen; C₁₋₄ alkyl optionally substituted by 1-4 groups R^{xx}; C₁₋₄ haloalkyl optionally substituted by 1-4 groups R^{xx}; C₁₋₄ alkoxy, C₁₋₄ alkylthio; or NR^{ac}R^{ad}. Most preferably Rⁱ is hydrogen, C₁₋₄ alkyl, C₁₋₄ alkoxy, C₁₋₄ alkylthio, or NR^{ac}R^{ad}.

R^{j} is optionally substituted alkyl or optionally substituted cycloalkyl. Preferably R^{j} is alkyl optionally substituted by one or more R^{ah} or cycloalkyl substituted by one of more R^{ai}. More preferably R^{j} is C₁₋₄ alkyl, or C₁₋₄ haloalkyl, and most preferably Rⁱ is C₁₋₄ alkyl.

R^{k} is hydrogen, optionally substituted alkyl provided said substitution does not comprise a ring system, optionally substituted amino, or optionally substituted alkylthio. Preferably R^{k} is: hydrogen; alkyl optionally substituted by one or more R^{xx}; NR^{ac}R^{ad}; or SR^{af}. More preferably R^{k} is: hydrogen; C₁₋₄ alkyl optionally substituted by 1-4 groups R^{xx}; C₁₋₄ haloalkyl optionally substituted by 1-4 groups R^{xx}; C₁₋₄ alkylthio; or NR^{ac}R^{ad}. Most preferably R^{k} is hydrogen, C₁₋₄ alkyl, C₁₋₄ alkylthio, or NR^{ac}R^{ad}.

R^{L} is optionally substituted alkyl, or optionally substituted cycloalkyl. Preferably R^{L} is alkyl optionally substituted by one or more R^{ah} or cycloalkyl optionally substituted by one or more R^{ai}. More preferably R^{L} is C₁₋₄ alkyl, or C₁₋₄ haloalkyl. Most preferably R^{L} is C₁₋₄ alkyl.

R^{m} is hydrogen, optionally substituted alkyl, optionally substituted cycloalkyl, optionally substituted phenyl, optionally substituted heteroaryl, or optionally substituted amino. Preferably R^{m} is: hydrogen; alkyl optionally substituted by one or more R^{xx}; cycloalkyl optionally substituted by one or more R^{yy}; phenyl optionally substituted by one or more R^{zz}; heteroaryl substituted by one or more R^{ab}; or NR^{ac}R^{ad}. More preferably R^{m} is: hydrogen; C₁₋₄ alkyl optionally substituted by 1-4 groups R^{xx}; C₁₋₄ haloalkyl optionally substituted by 1-4 groups R^{xx}; C₃₋₆ cycloalkyl optionally substituted by 1-4 groups R^{yy}: phenyl optionally substituted by 1-3 groups R^{zz}; or NR^{ac}R^{ad}. Most preferably R^{m} is hydrogen, C₁₋₄ alkyl, C₃₋₆ cycloalkyl, phenyl, or NR^{ac}R^{ad}.

Rⁿ and R^{o} are each independently hydrogen, optionally substituted alkyl or optionally substituted cycloalkyl. Preferably Rⁿ and R^{o} are each independently hydrogen, alkyl optionally substituted by one or more R^{ah}, or cycloalkyl optionally substituted by one or more R^{ai}. More preferably Rⁿ and R^{o} are each independently hydrogen, C₁₋₄ alkyl, or C₁₋₄ haloalkyl. Most preferably Rⁿ and R^{o} are each independently hydrogen, or C₁₋₄ alkyl.

R^{p} is halogen, cyano, nitro, hydroxy, alkoxy, alkoxyalkoxy, S(O)_{b}R^{am}, C(O)R^{an}, or NR^{ao}R^{ap}. Preferably R^{p} is halogen, cyano, hydroxy, C₁₋₄ alkoxy, or C₂₋₈ alkoxyalkoxy. More preferably R^{p} is halogen, hydroxy, or C₁₋₄ alkoxy.

R^{t} is halogen, cyano, or alkoxycarbonyl.

R^{u} is halogen, cyano, alkoxy, or alkoxycarbonyl;

Each R^{v} is independently: halogen; cyano; nitro; hydroxy; alkyl optionally substituted by one or more R^{aq}; alkenyl optionally substituted by one or more R^{ar}; alkynyl optionally substituted by one or more R^{as}; cycloalkyl substituted by one or more R^{al}; OR^{al}; S(O)_{b}R^{am}; C(O)R^{an}; or NR^{au}R^{av}; or any two geminal groups R^{v} together form an oxo group. Preferably each R^{v} is independently: halogen; cyano; hydroxy; C₁₋₄ alkyl; C₁₋₄ haloalkyl; C₂₋₈alkoxyalkyl; C₂₋₄ alkenyl optionally substituted by one or more R^{ar}; C₁₋₄ alkoxy; C₁₋₄ haloalkoxy; C₁₋₄ alkylsulphonyl; C₁₋₄ haloalkylsulphonyl; or C(O)R^{an}; or any two geminal groups R^{v} together form an oxo group. Most preferably each R^{v} is independently halogen, or C₁₋₄ alkyl;

Each R^{rr} is hydrogen, alkyl, haloalkyl, cycloalkyl, alkylcarbonyl, haloalkylcarbonyl, alkoxycarbonyl, aminocarbonyl, alkylaminocarbonyl, dialkylaminocarbonyl, or phenyl optionally substituted by one or more groups R^{zz}.

Each R^{ss} is alkyl, haloalkyl, alkenyl, haloalkenyl, alkynyl, haloalkynyl, cycloalkyl, or phenyl optionally substituted by one or more groups R^{zz}. Preferably R^{ss} is C₁₋₄ alkyl, phenyl optionally substituted with 1-3 groups R^{zz}, or C₁₋₄ haloalkyl. More preferably R^{ss} is C₁₋₄ alkyl, or phenyl optionally substituted with 1-3 groups R^{zz}.

Each R^{uu} is: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; NR^{ac}R^{ad}; or OR^{ae}. Preferably R^{uu} is: hydrogen; C₁₋₄ alkyl optionally substituted by 1-4 groups R^{xx}; C₁₋₄ haloalkyl optionally substituted by 1-4 groups R^{xx}; C₃₋₆ cycloalkyl optionally substituted by 1-4 groups R^{yy}, phenyl optionally substituted by 1-3 groups R^{zz}; heteroaryl optionally substituted by 1-3 groups R^{ab}; NR^{ac}R^{ad}; or OR^{ae}. More preferably R^{uu} is: C₁₋₄ alkyl; phenyl optionally substituted with 1-3 groups R^{zz}; C₁₋₄ alkoxy; or NR^{ac}R^{ad}.

Each R^{vv} is independently: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; SO₂R^{ss}; or C(O)R^{uu}.

Each R^{ww} is independently: hydrogen, alkyl optionally substituted by one or more R^{xx}; cycloalkyl optionally substituted by one or more R^{yy}; phenyl optionally substituted by one or more R^{zz}; or heteroaryl optionally substituted by one or more R^{ab}; or R^{vv} and R^{ww} together with the N atom to which they are attached form a 5-6 membered ring, said ring optionally containing one further heteroatom selected from O, S and N and being optionally substituted by 1-2 groups selected from alkyl and alkylcarbonyl.

Each R^{xx} is independently halogen, cyano, alkoxy, or alkoxycarbonyl. Preferably each R^{xx} is independently cyano, C₁₋₄ alkoxy, or C₁₋₄ alkoxycarbonyl.

Each R^{yy} is independently: halogen; cyano; alkyl; cycloalkyl; phenyl optionally substituted by one or more R^{zz}; heteroaryl optionally substituted by one or more R^{ab}; or alkoxycarbonyl. Preferably each R^{yy} is independently halogen, cyano or C₁₋₄ alkyl.

Each R^{zz} is independently: halogen; cyano; nitro; hydroxy; alkyl optionally substituted by one or more R^{aq}; alkenyl optionally substituted by one or more R^{ar}, alkynyl optionally substituted by one or more R^{as}; OR^{al}, S(O)_{b}R^{am}; or C(O)R^{an}. Preferably each R^{zz} is independently halogen, cyano, nitro, C₁₋₄alkyl, C₁₋₄ haloalkyl, C₂₋₈alkoxyalkyl, C₁₋₄ alkoxy, C₁₋₄haloalkoxy, C₁₋₄alkylsulphonyl, C₁₋₄haloalkylsulphonyl or C(O)R^{an}. More preferably R^{zz} is halogen, C₁₋₄ alkyl, C₁₋₄ alkoxy, or C₁₋₄ alkylsulphonyl.

Each R^{ab} is independently: halogen; cyano; hydroxy; alkyl optionally substituted by one or more R^{aq}; alkenyl optionally substituted by one or more R^{ar}, alkynyl optionally substituted by one or more R^{as}; OR^{al}, S(O)_{b}R^{am}; or C(O)R^{an}. Preferably each R^{ab} is independently halogen, cyano, nitro, C₁₋₄alkyl, C₁₋₄ haloalkyl, C₂₋₈alkoxyalkyl, C₁₋₄ alkoxy, C₁₋₄haloalkoxy, C₁₋₄alkylsulphonyl, C₁₋₄haloalkylsulphonyl, or C(O)R^{an}.

R^{ac} is hydrogen, alkyl, haloalkyl, alkoxyalkyl, alkylcarbonyl, alkoxycarbonyl, or phenyl optionally substituted by one or more R^{zz}. Preferably R^{ac} is hydrogen, C₁₋₄ alkyl, C₁₋₄ haloalkyl, or C₂₋₈ alkoxyalkyl. More preferably R^{ac} is hydrogen or C₁₋₄ alkyl.

R^{ad} is hydrogen, alkyl, haloalkyl, alkoxyalkyl, or phenyl optionally substituted by one or more R^{zz}. Preferably R^{ad} is hydrogen, C₁₋₄ alkyl, C₁₋₄ haloalkyl, or C₂₋₈ alkoxyalkyl. More preferably R^{ad} is hydrogen or C₁₋₄ alkyl;

R^{ae} is alkyl, phenylalkyl optionally substituted by one or more R^{zz}, or phenyl optionally substituted by one or more R^{zz}. Preferably R^{ae} is: C₁₋₄ alkyl; phenylC₁₋₄alkyl optionally substituted by 1-3 groups R^{zz}; or phenyl optionally substituted by 1-3 groups R^{zz}.

R^{af} is alkyl or phenyl.

Each R^{ag} is independently alkyl or haloalkyl.

Each R^{ah} is independently halogen or phenyl.

Each R^{ai} is independently halogen or alkyl.

R^{al} is alkyl, haloalkyl, alkoxyalkyl, or alkylsulphonyl.

R^{am} is alkyl, haloalkyl, alkenyl, alkynyl, phenyl, or alkylcarbonylamino.

b is an integer selected from 0, 1, and 2.

R^{an} is hydrogen, alkyl, haloalkyl, alkoxy, haloalkoxy, amino, alkylamino, dialkylamino, or alkylsulphonylamino. Preferably R^{an} is C₁₋₄ alkyl, C₁₋₄ haloalkyl, C₁₋₄ alkoxy, C₁₋₄ haloalkoxy, amino, C₁₋₄ alkylamino, or C₂₋₈ dialkylamino.

R^{ao} is hydrogen, alkyl, alkoxyalkyl, phenylalkyl, formyl, alkylcarbonyl, alkoxycarbonyl, aminocarbonyl, alkylaminocarbonyl, dialkylaminocarbonyl.

R^{ap} is hydrogen, alkyl, alkoxyalkyl, or phenylalkyl.

Each R^{aq} is independently halogen, alkoxy, cyano, alkoxycarbonyl, or alkylsulphonyl.

Each R^{ar} is independently halogen, cyano, nitro, alkoxy, alkylcarbonyl, alkoxycarbonyl, aminocarbonyl, alkylsulphonyl, or dialkyl phosphonyl. Preferably each each R^{ar} is independently halogen, cyano, nitro, or C₁₋₄ alkoxycarbonyl.

Each R^{as} is independently halogen, cyano, trialkylsilyl, alkylcarbonyl, alkoxycarbonyl, aminocarbonyl, or alkylsulphonyl.

Each R^{at} is independently halogen, alkyl, or alkoxycarbonyl.

R^{au} is hydrogen, alkyl, haloalkyl, alkoxyalkyl, alkylcarbonyl, alkoxycarbonyl, aminocarbonyl, or phenyl optionally substituted by one or more R^{zz}.

R^{av} is hydrogen, alkyl, haloalkyl, or alkoxyalkyl. Alternatively R^{au} and R^{av} together with the N atom to which they are attached form a 5-6 membered ring, said ring optionally containing one further heteroatom selected from O, S and N and being optionally substituted by 1-2 groups selected from alkyl and alkylcarbonyl;

According to the invention Y is defined as halogen, cyano, optionally substituted alkyl, optionally substituted cycloalkyl, optionally substituted alkenyl, optionally substituted alkynyl, optionally substituted phenyl, optionally substituted heteroaryl, optionally substituted alkoxy, optionally substituted amino, optionally substituted alkylthio, optionally substituted alkylsulphinyl, optionally substituted alkylsulphonyl, optionally substituted dialkyl phosphonyl, or optionally substituted trialkylsilyl.

Preferably Y is halogen; cyano; C₁₋₆ alkyl optionally substituted by 1-3 groups R^{ba}; C₁₋₆ haloalkyl optionally substituted by 1-3 groups R^{ba}; C₃₋₆ cycloalkyl optionally substituted by 1-3 groups R^{bc}; C₂₋₆ alkenyl optionally substituted by 1-3 groups R^{bd}; C₂₋₆ alkynyl optionally substituted by 1-3 groups R^{be}; phenyl optionally substituted by 1-4 groups R^{bf}; heteroaryl optionally substituted by 1-3 groups R^{bg}; OR^{bh}; NR^{bi}R^{bj}; S(O)_{c}R^{bk}; C₂₋₈dialkyl phosphonyl; or C₃₋₁₂-trialkylsilyl.

More preferably Y is halogen, C₁₋₃ alkyl, C₁₋₃ haloalkyl, C₂₋₅ alkoxyalkyl, cyclopropyl optionally substituted by 1-2 groups R^{bc}, C₂₋₄ alkenyl, C₂₋₄ haloalkenyl, or C₂₋₄ alkynyl optionally substituted by 1-2 groups R^{be};

Most preferably Y is halogen, C₁₋₂ alkyl, C₁₋₂ haloalkyl, C₂₋₃ alkoxyalkyl, or C₂₋₃ alkenyl.

Each R^{ba} is independently: cyano; nitro; hydroxy; C₁₋₄ alkoxy; C₁₋₄ alkylthio; C₁₋₄ alkylcarbonyl; or C₁₋₄ alkoxycarbonyl.

Each R^{bc} is independently: halogen; cyano; C₁₋₄ alkyl; C₁₋₄ alkoxy; or C₁₋₄ alkoxycarbonyl. Preferably each R^{bc} is independently halogen or C₁₋₂ alkyl.

Each R^{bd} is independently: halogen; cyano; C₁₋₄ alkylcarbonyl; or C₁₋₄ alkoxycarbonyl.

Each R^{be} is independently: halogen; cyano; hydroxy; C₁₋₄ alkoxycarbonyl; or tri(C₁₋₄)alkylsilyl (also referred to herein as C₃₋₁₂-trialkylsilyl). Preferably each R^{be} is independently halogen or tri(C₁₋₃)alkylsilyl(C₃₋₉trialkylsilyl).

Each R^{bf} is independently: halogen; cyano; C₁₋₄ alkyl; C₁₋₄ haloalkyl; C₁₋₄ alkoxy(C₁₋₄)alkyl (also referred to as C₂₋₈alkoxyalkyl); C₁₋₄ alkoxy; C₁₋₄ haloalkoxy; C₁₋₄ alkylsulphonyl; or C₁₋₄ alkoxycarbonyl.

Each R^{bg} is independently: halogen; cyano; C₁₋₄ alkyl; C₁₋₄ haloalkyl; C₁₋₄ alkoxy(C₁₋₄)alkyl (also referred to as C₂₋₈alkoxyalkyl); C₁₋₄ alkoxy; C₁₋₄ haloalkoxy; C₁₋₄ alkylsulphonyl; or C₁₋₄ alkoxycarbonyl.

Each R^{bh} is independently: C₁₋₄ alkyl; C₁₋₄ haloalkyl; or C₁₋₄ alkylsulphonyl.

Each R^{bi} is independently: hydrogen; C₁₋₄ alkyl; C₁₋₄ alkylcarbonyl; C₂₋₄ alkoxycarbonyl; aminocarbonyl; C₁₋₄ alkylaminocarbonyl; or di(C₁₋₄)alkylaminocarbonyl (C₂₋₈ dialkylaminocarbonyl).

Each R^{bj} is independently hydrogen or C₁₋₄ alkyl.

Each R^{bk} is independently C₁₋₄ alkyl or C₁₋₄ haloalkyl;

c is an integer selected from 0, 1 and 2.

According to the invention Z is defined as (T)ₘ-(U)ₙ—V. Preferably Z is (O)ₘ—(U)ₙ—V, more preferably Z is (O)ₘ—(CR^{w}R^{x})ₙ—C(O)R^{cb}, and most preferably Z is C(O)R^{cb}.

T is an oxygen or sulphur atom, preferably an oxygen atom.

m is an integer of 0 to 1; n is an integer selected from 0, 1, 2, and 3, and n≧m. Preferably n has the value of 0 or 1

U is CR^{w}R^{x}. Each R^{w} is independently hydrogen, halogen, hydroxy, optionally substituted alkyl, optionally substituted alkoxycarbonyl, or OR^{y}.

Each R^{x} is independently hydrogen, halogen, optionally substituted alkyl, or OR^{Z}.

Alternatively, any geminal R^{w} and R^{x} together form a group selected from oxo, or ═NOR^{ca}; or any geminal, vicinal or non-adjacent R^{w} and/or R^{x} together with the C atom(s) to which they are attached and any intervening atom form an optionally substituted 3-6 membered ring.

R^{ca} is hydrogen or optionally substituted alkyl.

Furthermore, when at least one R^{w} is OR^{y} and at least one R^{x} is OR^{Z}, said OR^{y} and OR^{Z} groups together with the C atom(s) to which they are attached and any intervening atom may form an optionally substituted 5-6 membered heterocyclic ring.

Preferably R^{w} is hydrogen, halogen, hydroxy, C₁₋₄ alkyl, C₁₋₄ haloalkyl, C₁₋₃ alkoxy(C₁₋₃)alkyl, C₁₋₆ alkoxycarbonyl, C₁₋₄ alkoxy, C₁₋₄ alkylcarbonyloxy.

More preferably R^{w} is hydrogen or C₁₋₄ alkyl.

Preferably R^{x} is hydrogen, halogen, C₁₋₄ alkyl, C₁₋₄ haloalkyl, C₁₋₃ alkoxy(C₁₋₃)alkyl, C₁₋₄ alkoxy; or any geminal R^{w} and R^{x} together form a group selected from oxo, or ═NOR^{ca}. More preferably R^{x} is hydrogen.

Each R^{y} is independently an optionally substituted alkyl or an optionally substituted alkylcarbonyl.

Each R^{Z} is independently an optionally substituted alkyl group.

According to the invention V is C(O)R^{cb}, C(S)R^{cd}, C(═NR^{ce})R^{cf}, CHR^{cg}R^{ch}, CH(S[O]ₚR^{ci})(S[O]_{q}R^{cj}), CR^{ck}R^{cl}R^{cm}, or CH₂OR^{cn}, wherein R^{cb}, R^{cd}, R^{ce}, R^{cf}, R^{cg}, R^{ch}, R^{ci}, R^{cj}, R^{ck}, R^{cl}, R^{cm} and R^{cn} are as defined below.

Each R^{cb} is hydrogen, hydroxy, optionally substituted alkoxy, optionally substituted cycloalkoxy, optionally substituted alkylthio, or optionally substituted amino. Preferably each R^{cb} is hydrogen, hydroxy, C₁₋₁₀ alkylthio, amino, C₁₋₆ alkylamino, di(C₁₋₄)alkylamino, or OR^{co}. More preferably each R^{cb} is hydroxy, C₁₋₁₀ alkylthio, or OR^{co}. Most preferably each R^{cb} is hydroxy, C₁₋₁₀alkoxy, or phenyl(C₁₋₂)alkoxy.

Each R^{cd} is optionally substituted alkoxy, optionally substituted cycloalkoxy, optionally substituted alkylthio, or optionally substituted amino. Preferably each R^{cd} is C₁₋₂₀ alkoxy, C₁₋₁₀ alkylthio, amino, C₁₋₆ alkylamino, or di(C₁₋₄)alkylamino.

Each R^{ce} is hydrogen, optionally substituted alkyl, optionally substituted alkoxy, optionally substituted cycloalkoxy, or optionally substituted amino. Preferably each R^{ce} is hydrogen, C₁₋₆ alkyl, amino, C₁₋₆ alkylamino, di(C₁₋₄alkylamino, C₁₋₆ alkoxy, or C₃₋₆ cycloalkoxy.

Each R^{cf} is hydrogen, optionally substituted alkoxy, optionally substituted cycloalkoxy, optionally substituted alkylthio, or optionally substituted amino. Preferably each R^{cf} is hydrogen, C₁₋₆ alkoxy, C₁₋₆ alkylthio, amino, C₁₋₆ alkylamino, or di(C₁₋₄)alkylamino.

R^{cg} and R^{ch} are each independently an optionally substituted alkoxy group. Alternatively R^{cg} and R^{ch}, together with the carbon to which they are joined, form a dioxolane or dioxane ring, which ring is optionally substituted, preferably by 1-2 C₁₋₂ alkyl groups. Preferably R^{cg} and R^{ch} are each independently C₁₋₄ alkoxy.

R^{ci} and R^{cj} are each independently an optionally substituted alkyl group. Alternatively R^{ci} and R^{cj}, together with the carbon and sulphur atoms to which they are joined, form an optionally substituted 5-6 membered ring, said ring is preferably substituted with 1 or 2 C₁₋₂ alkyl groups. Preferably R^{ci} and R^{cj} are each independently C₁₋₄ alkyl.

p and q are each independently an integer of 0, 1, or 2. Preferably p and q are each independently 0 or 1.

R^{ck}, R^{cl}, and R^{cm} are each independently an optionally substituted alkoxy group. Alternatively, R^{ck} and R^{cl} and R^{cm} together with the carbon to which they are attached form an optionally substituted trioxabicyclo[2.2.2]octane ring system. Preferably R^{ck}, R^{cl}, and R^{cm} are each independently C₁₋₄ alkoxy.

R^{cn} is hydrogen or an optionally substituted alkylcarbonyl group. Preferably R^{cn} is hydrogen or C₁₋₄alkylcarbonyl.

R^{co} is: C₁₋₂₀ alkyl optionally substituted by 1-3 groups R^{cq}; C₁₋₂₀ haloalkyl optionally substituted by 1-3 groups R^{cq}; or C₃₋₆ cycloalkyl. Preferably R^{co} is C₁₋₂₀ alkyl optionally substituted by 1-2 groups R^{cq} or C₁₋₂₀ haloalkyl optionally substituted by 1-2 groups R^{cq}.

Each R^{cq} is independently: C₃₋₆ cycloalkyl, C₁₋₆ alkoxy, phenyl optionally substituted with 1-4 groups R^{cr}, or heteroaryl optionally substituted with 1-3 groups R^{cs}. Preferably each R^{cq} is independently phenyl optionally substituted with 1-3 groups R^{cr}, or heteroaryl optionally substituted with 1-2 groups R^{cs}.

Each R^{cr} is independently: halogen, cyano, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, C₁₋₄ alkoxycarbonyl, or amino. Preferably each R^{cr} is independently halogen, cyano, C₁₋₄alkyl, C₁₋₄ haloalkyl, C₁₋₃ alkoxy(C₁₋₃)alkyl, C₁₋₄alkoxy, C₁₋₄ haloalkoxy, C₁₋₄ alkylsulphonyl, or C₁₋₄ alkoxycarbonyl.

Each R^{cs} is independently: halogen, cyano, C₁₋₄haloalkyl, C₁₋₃ alkoxy(C₁₋₃)alkyl, C₁₋₄ alkoxy, C₁₋₄ haloalkoxy, C₁₋₄ alkylsulphonyl, C₁₋₄ alkoxycarbonyl, or amino. Preferably each R^{cs} is independently halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, or C₁₋₄ alkoxycarbonyl.

Certain compounds of formula (I) are novel. Thus, in a further aspect the invention provides novel compounds of formula (I), which are compounds of formula (IA)

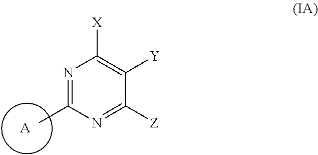

wherein A, Y, and Z are as defined hereinbefore in respect of compounds of formula (I), X is NR⁵R⁶ or halogen, and R⁵ and R⁶ are as defined hereinbefore in respect of compounds of formula (I). Preferred substituents for A, Y, Z, R⁵ and R⁶, in any combination, are as defined above in respect of compounds of formula (I).

Certain particularly preferred compounds of formula (IA) are those wherein A is selected from the group of substituents given in Table 1 below, and/or X is selected from C₁, NH₂, NHCH₃, N(CH₃)₂, NH(COCH₃), NH(CO₂CH₃), NH(SO₂CH₃), NCH₃(COCH₃), NCH₃(SO₂CH₃) and NH(CH[CH₃]₂), and/or Y is selected from the group consisting of Cl, Br and vinyl, and/or Z is selected from the group consisting of CO₂H, CO₂CH₃, and CO₂CH₂CH₃.

Even more preferred compounds of formula (IA) are those wherein A is selected from the group consisting of optionally substituted thiophene, optionally substituted pyrazole, optionally substituted pyridine, optionally substituted pyrimidine, optionally substituted benzothiophene, optionally substituted isoxazole, and optionally substituted isoquinoline, and/or X is selected from NH₂ or Cl, and/or Y is selected from the group consisting of Cl and vinyl, and/or Z is selected from the group consisting of CO₂H, CO₂CH₃, and CO₂CH₂CH₃.

The compounds described below are illustrative of novel compounds of the invention. Table 1 below provides 196 compounds designated as 1-1 to 1-196 respectively, of formula (I) wherein X is NH₂, Y is Cl, Z is CO₂H and wherein A has the value given in the Table.

TABLE 1

| Compound Number | A |
|---|---|
| 1-1 | thiophen-2-yl |
| 1-2 | 5-chlorothiophen-2-yl |
| 1-3 | 5-cyanothiophen-2-yl |
| 1-4 | 5-methylthiophen-2-yl |
| 1-5 | 5-bromothiophen-2-yl |
| 1-6 | 3-methylthiophen-2-yl |
| 1-7 | 5-methoxythiophen-2-yl |
| 1-8 | 4-cyanothiophen-2-yl |
| 1-9 | 5-methylcarbonylthiophen-2-yl |
| 1-10 | 4-trimethylsilylthiophen-2-yl |
| 1-11 | 5-trimethylsilylthiophen-2-yl |
| 1-12 | 4-bromo-5-chlorothiophen-2-yl |
| 1-13 | 4-bromothiophen-2-yl |
| 1-14 | 4-chlorothiophen-2-yl |
| 1-15 | 4-methylthiophen-2-yl |
| 1-16 | thiophen-3-yl |
| 1-17 | 4-methylthiophen-3-yl |
| 1-18 | 2-chlorothiophen-3-yl |
| 1-19 | 2,5-dichlorothiophen-3-yl |
| 1-20 | 2-cyanothiophen-3-yl |
| 1-21 | 2-methylthiophen-3-yl |
| 1-22 | 5-bromo-2-chlorothiophen-3-yl |
| 1-23 | 5-bromo-2-cyanothiophen-3-yl |
| 1-24 | 4-bromo-2,5-dimethylthiophen-3-yl |
| 1-25 | 2-bromothiophen-3-yl |
| 1-26 | 2-chloro-5-trimethylsilylthiophen-3-yl |
| 1-27 | 5-methylthiophen-3-yl |
| 1-28 | 5-chlorothiophen-3-yl |
| 1-29 | furan-2-yl |
| 1-30 | 5-methylfuran-2-yl |
| 1-31 | 5-chlorofuran-2-yl |
| 1-32 | 4-chlorofuran-2-yl |
| 1-33 | 4-methylfuran-2-yl |
| 1-34 | furan-3-yl |
| 1-35 | 2-cyano-5-methylfuran-3-yl |
| 1-36 | 5-methylfuran-3-yl |
| 1-37 | 5-chlorofuran-3-yl |
| 1-38 | pyrrol-1-yl |
| 1-39 | 3-methylpyrrol-1-yl |
| 1-40 | 3-chloropyrrol-1-yl |
| 1-41 | pyrrol-2-yl |
| 1-42 | 4-methylcarbonylpyrrol-2-yl |
| 1-43 | 4-methylpyrrol-2-yl |
| 1-44 | 4-chloropyrrol-2-yl |
| 1-45 | 5-chloropyrrol-2-yl |
| 1-46 | 5-methylpyrrol-2-yl |
| 1-47 | pyrrol-3-yl |
| 1-48 | 1-triisopropylsilylpyrrol-3-yl |
| 1-49 | 1-tert-butoxycarbonylpyrrol-3-yl |
| 1-50 | 5-chloropyrrol-3-yl |
| 1-51 | 5-methylpyrrol-3-yl |
| 1-52 | 1-methylpyrrol-3-yl |
| 1-53 | benzofuran-2-yl |
| 1-54 | benzothiophen-2-yl |
| 1-55 | 5-methylbenzothiophen-2-yl |
| 1-56 | benzothiophen-3-yl |
| 1-57 | 5-methylbenzothiophen-3-yl |
| 1-58 | indol-2-yl |
| 1-59 | 1-methylindol-2-yl |
| 1-60 | 1-tert-butoxycarbonylindol-2-yl |
| 1-61 | 1-phenylsulphonylindol-2-yl |
| 1-62 | 1-phenylsulphonylindol-3-yl |
| 1-63 | 1-tert-butoxycarbonylindol-3-yl |
| 1-64 | indol-3-yl |
| 1-65 | pyrazol-1-yl |
| 1-66 | 3-methylpyrazol-1-yl |
| 1-67 | 3-chloropyrazol-1-yl |
| 1-68 | 4-methylpyrazol-1-yl |
| 1-69 | 4-chloropyrazol-1-yl |
| 1-70 | pyrazol-4-yl |
| 1-71 | 1-methylpyrazol-4-yl |
| 1-72 | 3,5-dimethylpyrazol-4-yl |
| 1-73 | 1,3,5-trimethylpyrazol-4-yl |
| 1-74 | 4-bromo-1-methylpyrazol-5-yl |
| 1-75 | 1-methylpyrazol-5-yl |
| 1-76 | 1-dimethylaminocarbonylpyrazol-4-yl |
| 1-77 | 1-methylpyrazol-3-yl |
| 1-78 | 5-methylpyrazol-3-yl |
| 1-79 | 5-chloropyrazol-3-yl |
| 1-80 | 5-chloro-1-methylpyrazol-3-yl |
| 1-81 | 1,5-dimethylpyrazol-3-yl |
| 1-82 | 3-methylisoxazol-5-yl |
| 1-83 | 3-chloroisoxazol-5-yl |
| 1-84 | 5-methylisoxazol-3-yl |
| 1-85 | 5-chloroisoxazol-3-yl |
| 1-86 | 3,5-dimethylisoxazol-4-yl |
| 1-87 | 3-methylisothiazol-5-yl |
| 1-88 | 3-chloroisothiazol-5-yl |
| 1-89 | 5-methylisothiazol-3-yl |
| 1-90 | 5-chloroisothiazol-3-yl |
| 1-91 | oxazol-5-yl |
| 1-92 | 2-methyloxazol-5-yl |
| 1-93 | 2-chlorooxazol-5-yl |
| 1-94 | 5-chlorooxazol-2-yl |
| 1-95 | 5-methyloxazol-2-yl |
| 1-96 | 2-methyloxazol-4-yl |
| 1-97 | 2-chlorooxazol-4-yl |
| 1-98 | 4-chlorooxazol-2-yl |
| 1-99 | 4-methyloxazol-2-yl |
| 1-100 | 2-methylthiazol-5-yl |
| 1-101 | 2-chlorothiazol-5-yl |
| 1-102 | 5-methylthiazol-2-yl |
| 1-103 | 5-chlorothiazol-2-yl |
| 1-104 | 2-methylthiazol-4-yl |
| 1-105 | 2-chlorothiazol-4-yl |
| 1-106 | 4-methylthiazol-2-yl |
| 1-107 | 4-chlorothiazol-2-yl |
| 1-108 | imidazol-1-yl |
| 1-109 | 4-methylimidazol-1-yl |
| 1-110 | 4-chloroimidazol-1-yl |
| 1-111 | 2-bromo-1-methylimidazol-5-yl |
| 1-112 | 1-methylimidazol-5-yl |
| 1-113 | 1-dimethylaminosulphonylimidazol-4-yl |
| 1-114 | 2-methylimidazol-4-yl |
| 1-115 | 2-chloroimidazol-4-yl |
| 1-116 | 4-methylimidazol-2-yl |
| 1-117 | 4-chloroimidazol-2-yl |
| 1-118 | 5-chloro-1,3,4-thiadiazol-2-yl |
| 1-119 | 5-methyl-1,3,4-thiadiazol-2-yl |
| 1-120 | 3-methyl-1,2,4-thiadiazol-5-yl |
| 1-121 | 3-chloro-1,2,4-thiadiazol-5-yl |
| 1-122 | 5-methyl-1,2,4-thiadiazol-3-yl |
| 1-123 | 5-chloro-1,2,4-thiadiazol-3-yl |
| 1-124 | 5-chloro-1,3,4-oxadiazol-2-yl |
| 1-125 | 5-methyl-1,3,4-oxadiazol-2-yl |
| 1-126 | 3-methyl-1,2,4-oxadiazol-5-yl |
| 1-127 | 3-chloro-1,2,4-oxadiazol-5-yl |
| 1-128 | 5-methyl-1,2,4-oxadiazol-3-yl |
| 1-129 | 5-chloro-1,2,4-oxadiazol-3-yl |
| 1-130 | 1,2,3-triazol-1-yl |
| 1-131 | 1,2,3-triazol-2-yl |
| 1-132 | 4-methyl-1,2,3-triazol-1-yl |
| 1-133 | 4-chloro-1,2,3-triazol-1-yl |
| 1-134 | 4-methyl-1,2,3-triazol-2-yl |
| 1-135 | 4-chloro-1,2,3-triazol-2-yl |
| 1-136 | 1,2,4-triazol-1-yl |
| 1-137 | 1,2,4-triazol-4-yl |
| 1-138 | 3-methyl-1,2,4-triazol-1-yl |
| 1-139 | 3-chloro-1,2,4-triazol-1-yl |
| 1-140 | 5-methyl-1,2,4-triazol-3-yl |
| 1-141 | 5-chloro-1,2,4-triazol-3-yl |
| 1-142 | 1-methyl-1,2,4-triazol-3-yl |
| 1-143 | 2-methyltetrazol-5-yl |
| 1-144 | 5-methyltetrazol-2-yl |
| 1-145 | 5-chlorotetrazol-2-yl |
| 1-146 | imidazo[1,5-a]pyridin-3-yl |
| 1-147 | 1-tert-butoxycarbonyl-1H-pyrrolo[2,3-b]pyridin-3-yl |
| 1-148 | 7-chloro-1H-pyrrolo[2,3-c]pyridin-2-yl |
| 1-149 | pyridin-2-yl |
| 1-150 | 5-trifluoromethylpyridin-2-yl |
| 1-151 | 5-chloropyridin-2-yl |
| 1-152 | 5-methylpyridin-2-yl |
| 1-153 | pyridin-3-yl |
| 1-154 | 2-methoxypyridin-3-yl |
| 1-155 | 6-methoxypyridin-3-yl |
| 1-156 | 6-chloropyridin-3-yl |
| 1-157 | 6-fluoropyridin-3-yl |
| 1-158 | 6-cyanopyridin-3-yl |
| 1-159 | 2,6-dimethoxypyridin-3-yl |
| 1-160 | 6-methylpyridin-3-yl |
| 1-161 | 2-fluoro-6-methylpyridin-3-yl |
| 1-162 | 4-methoxypyridin-3-yl |
| 1-163 | 6-aminopyridin-3-yl |
| 1-164 | 6-(piperidin-1-yl)-pyridin-3-yl |
| 1-165 | 4-trifluoromethylpyridin-3-yl |
| 1-166 | 2-(2,2,2-trifluoroethoxy)-pyridin-3-yl |
| 1-167 | 6-bromopyridin-3-yl |
| 1-168 | 6-nitropyridin-3-yl |
| 1-169 | 6-(piperazin-1-yl)-pyridin-3-yl |
| 1-170 | 6-(morpholin-1-yl)-pyridin-3-yl |
| 1-171 | 5-bromopyridin-3-yl |
| 1-172 | 2-fluoropyridin-3-yl |
| 1-173 | 6-methylcarbonylaminopyridin-3-yl |
| 1-174 | pyridin-4-yl |
| 1-175 | 3-chloropyridin-4-yl |
| 1-176 | 3,5-difluoropyridin-4-yl |
| 1-177 | 2,3-dichloropyridin-4-yl |
| 1-178 | 6-(piperazin-1-yl)-pyridin-4-yl |
| 1-179 | 2,6-dichloropyridin-4-yl |
| 1-180 | 5-methylpyrimidin-2-yl |
| 1-181 | 5-chloropyrimidin-2-yl |
| 1-182 | 2-methoxypyrimidin-5-yl |
| 1-183 | 2,4-dimethoxypyrimidin-5-yl |
| 1-184 | 2-methylpyrimidin-5-yl |
| 1-185 | 2-chloropyrimidin-5-yl |
| 1-186 | 6-chloropyrazin-3-yl |
| 1-187 | 6-methylpyrazin-3-yl |
| 1-188 | 5-methylpyridazin-2-yl |
| 1-189 | 5-chloropyridazin-2-yl |
| 1-190 | 6-methyl-1,2,4-triazin-3-yl |
| 1-191 | 6-chloro-1,2,4-triazin-3-yl |
| 1-192 | 3-methyl-1,2,4-triazin-6-yl |
| 1-193 | 3-chloro-1,2,4-triazin-6-yl |
| 1-194 | 6-methyl-1,2,4,5-tetrazin-3-yl |
| 1-195 | quinolin-3-yl |
| 1-196 | isoquinolin-4-yl |

196 compounds of formula (I), wherein X is NH₂, Y is Cl, Z is CO₂CH₃ and the value of A is as given in Table 1 for compounds 1-1 to 1-196, are designated as compound Nos. 2-1 to 2-196 respectively.

196 compounds of formula (I), wherein X is NH₂, Y is Cl, Z is CO₂CH₂CH₃ and the value of A is as given in Table 1 for compounds 1-1 to 1-196, are designated as compound Nos. 3-1 to 3-196 respectively.

196 compounds of formula (I), wherein X is NH₂, Y is Br, Z is CO₂H and the value of A is as given in Table 1 for compounds 1-1 to 1-196, are designated as compound Nos. 4-1 to 4-196 respectively.

196 compounds of formula (I), wherein X is NH₂, Y is Br, Z is CO₂CH₃ and the value of A is as given in Table 1 for compounds 1-1 to 1-196, are designated as compound Nos. 5-1 to 5-196 respectively.

196 compounds of formula (I), wherein X is NH₂, Y is Br, Z is CO₂CH₂CH₃ and the value of A is as given in Table 1 for compounds 1-1 to 1-196, are designated as compound Nos. 6-1 to 6-196 respectively.

196 compounds of formula (I), wherein X is NHCH₃, Y is Cl, Z is CO₂H and the value of A is as given in Table 1 for compounds 1-1 to 1-196, are designated as compound Nos. 7-1 to 7-196 respectively.

196 compounds of formula (I), wherein X is NHCH₃, Y is Cl, Z is CO₂CH₃ and the value of A is as given in Table 1 for compounds 1-1 to 1-196, are designated as compound Nos. 8-1 to 8-196 respectively.

196 compounds of formula (I), wherein X is NHCH₃, Y is Cl, Z is CO₂CH₂CH₃ and the value of A is as given in Table 1 for compounds 1-1 to 1-196, are designated as compound Nos. 9-1 to 9-196 respectively.

196 compounds of formula (I), wherein X is NHCH₃, Y is Br, Z is CO₂H and the value of A is as given in Table 1 for compounds 1-1 to 1-196, are designated as compound Nos. 10-1 to 10-196 respectively.

196 compounds of formula (I), wherein X is NHCH₃, Y is Br, Z is CO₂CH₃ and the value of A is as given in Table 1 for compounds 1-1 to 1-196, are designated as compound Nos. 11-1 to 11-196 respectively.

196 compounds of formula (I), wherein X is NHCH₃, Y is Br, Z is CO₂CH₂CH₃ and the value of A is as given in Table 1 for compounds 1-1 to 1-196, are designated as compound Nos. 12-1 to 12-196 respectively.

196 compounds of formula (I), wherein X is N(CH₃)₂, Y is Cl, Z is CO₂H and the value of A is as given in Table 1 for compounds 1-1 to 1-196, are designated as compound Nos. 13-1 to 13-196 respectively.

196 compounds of formula (I), wherein X is N(CH₃)₂, Y is Cl, Z is CO₂CH₃ and the value of A is as given in Table 1 for compounds 1-1 to 1-196, are designated as compound Nos. 14-1 to 14-196 respectively.

196 compounds of formula (I), wherein X is N(CH₃)₂, Y is Cl, Z is CO₂CH₂CH₃ and the value of A is as given in Table 1 for compounds 1-1 to 1-196, are designated as compound Nos. 15-1 to 15-196 respectively.

196 compounds of formula (I), wherein X is N(CH₃)₂, Y is Br, Z is CO₂H and the value of A is as given in Table 1 for compounds 1-1 to 1-196, are designated as compound Nos. 16-1 to 16-196 respectively.

196 compounds of formula (I), wherein X is N(CH₃)₂, Y is Br, Z is CO₂CH₃ and the value of A is as given in Table 1 for compounds 1-1 to 1-196, are designated as compound Nos. 17-1 to 17-196 respectively.

196 compounds of formula (I), wherein X is N(CH₃)₂, Y is Br, Z is CO₂CH₂CH₃ and the value of A is as given in Table 1 for compounds 1-1 to 1-196, are designated as compound Nos. 18-1 to 18-196 respectively.

196 compounds of formula (I), wherein X is NH(COCH₃), Y is Cl, Z is CO₂H and the value of A is as given in Table 1 for compounds 1-1 to 1-196, are designated as compound Nos. 19-1 to 19-196 respectively.

196 compounds of formula (I), wherein X is NH(COCH₃), Y is Cl, Z is CO₂CH₃ and the value of A is as given in Table 1 for compounds 1-1 to 1-196, are designated as compound Nos. 20-1 to 20-196 respectively.

196 compounds of formula (I), wherein X is NH(COCH₃), Y is Cl, Z is CO₂CH₂CH₃ and the value of A is as given in Table 1 for compounds 1-1 to 1-196, are designated as compound Nos. 21-1 to 21-196 respectively.

196 compounds of formula (I), wherein X is NH(COCH₃), Y is Br, Z is CO₂H and the value of A is as given in Table 1 for compounds 1-1 to 1-196, are designated as compound Nos. 22-1 to 22-196 respectively.

196 compounds of formula (I), wherein X is NH(COCH₃), Y is Br, Z is CO₂CH₃ and the value of A is as given in Table 1 for compounds 1-1 to 1-196, are designated as compound Nos. 23-1 to 23-196 respectively.

196 compounds of formula (I), wherein X is NH(COCH₃), Y is Br, Z is CO₂CH₂CH₃ and the value of A is as given in Table 1 for compounds 1-1 to 1-196, are designated as compound Nos. 24-1 to 24-196 respectively.

196 compounds of formula (I), wherein X is NH(CO₂CH₃), Y is Cl, Z is CO₂H and the value of A is as given in Table 1 for compounds 1-1 to 1-196, are designated as compound Nos. 25-1 to 25-196 respectively.

196 compounds of formula (I), wherein X is NH(CO₂CH₃), Y is Cl, Z is CO₂CH₃ and the value of A is as given in Table 1 for compounds 1-1 to 1-196, are designated as compound Nos. 26-1 to 26-196 respectively.

196 compounds of formula (I), wherein X is NH(CO₂CH₃), Y is Cl, Z is CO₂CH₂CH₃ and the value of A is as given in Table 1 for compounds 1-1 to 1-196, are designated as compound Nos. 27-1 to 27-196 respectively.

196 compounds of formula (I), wherein X is NH(CO₂CH₃), Y is Br, Z is CO₂H and the value of A is as given in Table 1 for compounds 1-1 to 1-196, are designated as compound Nos. 28-1 to 28-196 respectively.

196 compounds of formula (I), wherein X is NH(CO₂CH₃), Y is Br, Z is CO₂CH₃ and the value of A is as given in Table 1 for compounds 1-1 to 1-196, are designated as compound Nos. 29-1 to 29-196 respectively.

196 compounds of formula (I), wherein X is NH(CO₂CH₃), Y is Br, Z is CO₂CH₂CH₃ and the value of A is as given in Table 1 for compounds 1-1 to 1-196, are designated as compound Nos. 30-1 to 30-196 respectively.

196 compounds of formula (I), wherein X is NH(SO₂CH₃), Y is Cl, Z is CO₂H and the value of A is as given in Table 1 for compounds 1-1 to 1-196, are designated as compound Nos. 31-1 to 31-196 respectively.

196 compounds of formula (I), wherein X is NH(SO₂CH₃), Y is Cl, Z is CO₂CH₃ and the value of A is as given in Table 1 for compounds 1-1 to 1-196, are designated as compound Nos. 32-1 to 32-196 respectively.

196 compounds of formula (I), wherein X is NH(SO₂CH₃), Y is Cl, Z is CO₂CH₂CH₃ and the value of A is as given in Table 1 for compounds 1-1 to 1-196, are designated as compound Nos. 33-1 to 33-196 respectively.

196 compounds of formula (I), wherein X is NH(SO₂CH₃), Y is Br, Z is CO₂H and the value of A is as given in Table 1 for compounds 1-1 to 1-196, are designated as compound Nos. 34-1 to 34-196 respectively.

196 compounds of formula (I), wherein X is NH(SO₂CH₃), Y is Br, Z is CO₂CH₃ and the value of A is as given in Table 1 for compounds 1-1 to 1-196, are designated as compound Nos. 35-1 to 35-196 respectively.

196 compounds of formula (I), wherein X is NH(SO₂CH₃), Y is Br, Z is CO₂CH₂CH₃ and the value of A is as given in Table 1 for compounds 1-1 to 1-196, are designated as compound Nos. 36-1 to 36-196 respectively.

196 compounds of formula (I), wherein X is NCH₃(COCH₃), Y is Cl, Z is CO₂H and the value of A is as given in Table 1 for compounds 1-1 to 1-196, are designated as compound Nos. 37-1 to 37-196 respectively.

196 compounds of formula (I), wherein X is NCH₃(COCH₃), Y is Cl, Z is CO₂CH₃ and the value of A is as given in Table 1 for compounds 1-1 to 1-196, are designated as compound Nos. 38-1 to 38-196 respectively.

196 compounds of formula (I), wherein X is NCH₃(COCH₃), Y is Cl, Z is CO₂CH₂CH₃ and the value of A is as given in Table 1 for compounds 1-1 to 1-196, are designated as compound Nos. 39-1 to 39-196 respectively.

196 compounds of formula (I), wherein X is NCH₃(COCH₃), Y is Br, Z is CO₂H and the value of A is as given in Table 1 for compounds 1-1 to 1-196, are designated as compound Nos. 40-1 to 40-196 respectively.

196 compounds of formula (I), wherein X is NCH₃(COCH₃), Y is Br, Z is CO₂CH₃ and the value of A is as given in Table 1 for compounds 1-1 to 1-196, are designated as compound Nos. 41-1 to 41-196 respectively.

196 compounds of formula (I), wherein X is NCH₃(COCH₃), Y is Br, Z is CO₂CH₂CH₃ and the value of A is as given in Table 1 for compounds 1-1 to 1-196, are designated as compound Nos. 42-1 to 42-196 respectively.

196 compounds of formula (I), wherein X is NCH₃(CO₂CH₃), Y is Cl, Z is CO₂H and the value of A is as given in Table 1 for compounds 1-1 to 1-196, are designated as compound Nos. 43-1 to 43-196 respectively.

196 compounds of formula (I), wherein X is NCH₃(CO₂CH₃), Y is Cl, Z is CO₂CH₃ and the value of A is as given in Table 1 for compounds 1-1 to 1-196, are designated as compound Nos. 44-1 to 44-196 respectively.

196 compounds of formula (I), wherein X is NCH₃(CO₂CH₃), Y is Cl, Z is CO₂CH₂CH₃ and the value of A is as given in Table 1 for compounds 1-1 to 1-196, are designated as compound Nos. 45-1 to 45-196 respectively.

196 compounds of formula (I), wherein X is NCH₃(CO₂CH₃), Y is Br, Z is CO₂H and the value of A is as given in Table 1 for compounds 1-1 to 1-196, are designated as compound Nos. 46-1 to 46-196 respectively.

196 compounds of formula (I), wherein X is NCH₃(CO₂CH₃), Y is Br, Z is CO₂CH₃ and the value of A is as given in Table 1 for compounds 1-1 to 1-196, are designated as compound Nos. 47-1 to 47-196 respectively.

196 compounds of formula (I), wherein X is NCH₃(CO₂CH₃), Y is Br, Z is CO₂CH₂CH₃ and the value of A is as given in Table 1 for compounds 1-1 to 1-196, are designated as compound Nos. 48-1 to 48-196 respectively.

196 compounds of formula (I), wherein X is NCH₃(SO₂CH₃), Y is Cl, Z is CO₂H and the value of A is as given in Table 1 for compounds 1-1 to 1-196, are designated as compound Nos. 49-1 to 49-196 respectively.

196 compounds of formula (I), wherein X is NCH₃(SO₂CH₃), Y is Cl, Z is CO₂CH₃ and the value of A is as given in Table 1 for compounds 1-1 to 1-196, are designated as compound Nos. 50-1 to 50-196 respectively.

196 compounds of formula (I), wherein X is NCH₃(SO₂CH₃), Y is Cl, Z is CO₂CH₂CH₃ and the value of A is as given in Table 1 for compounds 1-1 to 1-196, are designated as compound Nos. 51-1 to 51-196 respectively.

196 compounds of formula (I), wherein X is NCH₃(SO₂CH₃), Y is Br, Z is CO₂H and the value of A is as given in Table 1 for compounds 1-1 to 1-196, are designated as compound Nos. 52-1 to 52-196 respectively.

196 compounds of formula (I), wherein X is NCH₃(SO₂CH₃), Y is Br, Z is CO₂CH₃ and the value of A is as given in Table 1 for compounds 1-1 to 1-196, are designated as compound Nos. 53-1 to 53-196 respectively.

196 compounds of formula (I), wherein X is NCH₃(SO₂CH₃), Y is Br, Z is CO₂CH₂CH₃ and the value of A is as given in Table 1 for compounds 1-1 to 1-196, are designated as compound Nos. 54-1 to 54-196 respectively.

196 compounds of formula (I), wherein X is NH(CH[CH₃]₂), Y is Cl, Z is CO₂H and the value of A is as given in Table 1 for compounds 1-1 to 1-196, are designated as compound Nos. 55-1 to 55-196 respectively.

196 compounds of formula (I), wherein X is NH(CH[CH₃]₂), Y is Cl, Z is CO₂CH₃ and the value of A is as given in Table 1 for compounds 1-1 to 1-196, are designated as compound Nos. 56-1 to 56-196 respectively.

196 compounds of formula (I), wherein X is NH(CH[CH₃]₂), Y is Cl, Z is CO₂CH₂CH₃ and the value of A is as given in Table 1 for compounds 1-1 to 1-196, are designated as compound Nos. 57-1 to 57-196 respectively.

196 compounds of formula (I), wherein X is NH(CH[CH₃]₂), Y is Br, Z is CO₂H and the value of A is as given in Table 1 for compounds 1-1 to 1-196, are designated as compound Nos. 58-1 to 58-196 respectively.

196 compounds of formula (I), wherein X is NH(CH[CH₃]₂), Y is Br, Z is CO₂CH₃ and the value of A is as given in Table 1 for compounds 1-1 to 1-196, are designated as compound Nos. 59-1 to 59-196 respectively.

196 compounds of formula (I), wherein X is NH(CH[CH₃]₂), Y is Br, Z is CO₂CH₂CH₃ and the value of A is as given in Table 1 for compounds 1-1 to 1-196, are designated as compound Nos. 60-1 to 60-196 respectively.

196 compounds of formula (I), wherein X is Cl Y is Cl, Z is CO₂H and the value of A is as given in Table 1 for compounds 1-1 to 1-196, are designated as compound Nos. 61-1 to 61-196 respectively.

196 compounds of formula (I), wherein X is Cl Y is Cl, Z is CO₂CH₃ and the value of A is as given in Table 1 for compounds 1-1 to 1-196, are designated as compound Nos. 62-1 to 62-196 respectively.

196 compounds of formula (I), wherein X is Cl Y is Cl, Z is CO₂CH₂CH₃ and the value of A is as given in Table 1 for compounds 1-1 to 1-196, are designated as compound Nos. 63-1 to 63-196 respectively.

196 compounds of formula (I), wherein X is Cl Y is Br, Z is CO₂H and the value of A is as given in Table 1 for compounds 1-1 to 1-196, are designated as compound Nos. 64-1 to 64-196 respectively.

196 compounds of formula (I), wherein X is Cl Y is Br, Z is CO₂CH₃ and the value of A is as given in Table 1 for compounds 1-1 to 1-196, are designated as compound Nos. 65-1 to 65-196 respectively.

196 compounds of formula (I), wherein X is Cl Y is Br, Z is CO₂CH₂CH₃ and the value of A is as given in Table 1 for compounds 1-1 to 1-196, are designated as compound Nos. 66-1 to 66-196 respectively.

196 compounds of formula (I), wherein X is NH₂, Y is vinyl, Z is CO₂H and the value of A is as given in Table 1 for compounds 1-1 to 1-196, are designated as compound Nos. 67-1 to 67-196 respectively.

196 compounds of formula (I), wherein X is NH₂, Y is vinyl, Z is CO₂CH₃ and the value of A is as given in Table 1 for compounds 1-1 to 1-196, are designated as compound Nos. 68-1 to 68-196 respectively.

196 compounds of formula (I), wherein X is NH₂, Y is vinyl, Z is CO₂CH₂CH₃ and the value of A is as given in Table 1 for compounds 1-1 to 1-196, are designated as compound Nos. 69-1 to 69-196 respectively.

196 compounds of formula (I), wherein X is NHCH₃, Y is vinyl, Z is CO₂H and the value of A is as given in Table 1 for compounds 1-1 to 1-196, are designated as compound Nos. 70-1 to 70-196 respectively.

196 compounds of formula (I), wherein X is NHCH₃, Y is vinyl, Z is CO₂CH₃ and the value of A is as given in Table 1 for compounds 1-1 to 1-196, are designated as compound Nos. 71-1 to 71-196 respectively.

196 compounds of formula (I), wherein X is NHCH₃, Y is vinyl, Z is CO₂CH₂CH₃ and the value of A is as given in Table 1 for compounds 1-1 to 1-196, are designated as compound Nos. 72-1 to 72-196 respectively.

196 compounds of formula (I), wherein X is N(CH₃)₂, Y is vinyl, Z is CO₂H and the value of A is as given in Table 1 for compounds 1-1 to 1-196, are designated as compound Nos. 73-1 to 73-196 respectively.

196 compounds of formula (I), wherein X is N(CH₃)₂, Y is vinyl, Z is CO₂CH₃ and the value of A is as given in Table 1 for compounds 1-1 to 1-196, are designated as compound Nos. 74-1 to 74-196 respectively.

196 compounds of formula (I), wherein X is N(CH₃)₂, Y is Br, Z is CO₂CH₂CH₃ and the value of A is as given in Table 1 for compounds 1-1 to 1-196, are designated as compound Nos. 75-1 to 75-196 respectively.

General methods for the production of compounds of formula (I) are described below. Unless otherwise stated in the text, the substituents A, X, Y and Z (including the definitions of U, V, T, m and n) are as defined hereinbefore. The abbreviation LG as used herein refers to any suitable leaving group, and includes halogen atoms, sulphonate, and sulphone groups. The groups R as used herein are, independently of each other, alkyl or substituted alkyl groups. The groups R′ may, independently of each other, take a range of values depending on the particular structure of the molecule in which they are present; the skilled man will recognise what values are applicable in each case, particularly in view of the definition of compounds of formula (I) as described hereinbefore.

Compounds of formula (I) may be prepared from compounds of formula A as shown in reaction scheme 1.

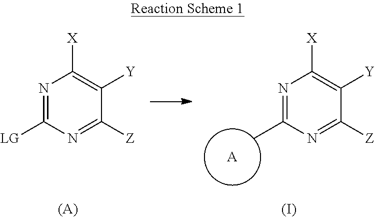

For example, a compound of formula (I) in which ring A is linked to the pyrimidine ring through a nitrogen atom may be prepared by reaction of a compound of formula (A) with (A-H) (for example pyrrole), optionally in the presence of a suitable base (for example an amine base, such as triethylamine), in a suitable solvent (for example an alcohol, such as methanol)—see reaction scheme 2 below. The reaction may be performed at ambient temperature or, preferably, at an elevated temperature. This transformation may also be performed in the presence of a suitable metal (for example palladium) catalyst, optionally complexed by suitable ligands (for example phosphine ligands, such as Josiphos).

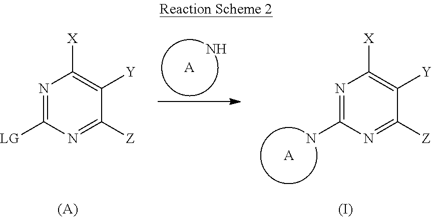

In a second example (see reaction scheme 3) a compound of formula (I) in which A is a group attached through a carbon atom may be prepared by reacting a suitable metal or metalloid derivative A-M (for example, a boronic acid or ester, a trialkyltin derivative, a zinc derivative or a Grignard reagent) with a compound of formula (A) in the presence of a suitable base (for example an inorganic base, such as potassium phosphate or caesium fluoride, or an organic base, such as triethylamine), a metal source (for example, a palladium source, such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakistriphenylphosphine, bis(triphenylphosphine)palladium dichloride or [1,1′-bis(diphenylphosphino)ferrocene]palladium dichloride).

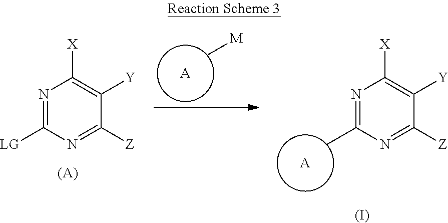

In a further example a compound of formula (I) may be prepared from a compound of formula (A1), which is a compound of formula A in which LG is an alkylthio group (see reaction scheme 4 below).

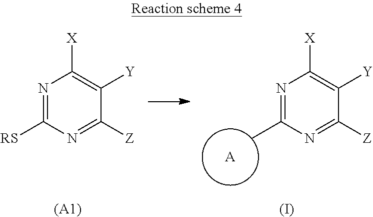

For example, such a transformation may be carried out by reaction with a metal or metalloid derivative of the ring A (for example a boronic acid or boronate ester) in the presence of a metal source (for example, a palladium source such as Pd₂(dba)₃) and, optionally, a ligand for the metal (for example a phosphine ligand, such as tri(2-furyl)phosphine), a further metal source (for example a copper complex, such as copper thiophene-2-carboxylate) in a suitable solvent (for example an ether, such as tetrahydrofuran). The metal catalyst and ligands may also be added as a single, pre-formed, complex (for example a palladium/phosphine complex, such as palladium tetrakistriphenylphosphine, bis(triphenylphosphine)palladium dichloride or [1,1′-bis(diphenylphosphino)ferrocene]palladium dichloride).

A compound of formula (A1) may be prepared by reacting a compound of formula (A) with an alkanethiolate (for example sodium methanethiolate) in a suitable solvent (for example a polar solvent, such as methanol). See reaction scheme 5 below.

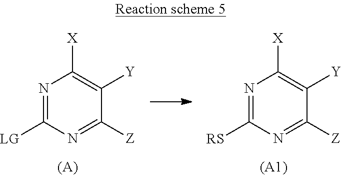

Compounds of formula (I) may also be prepared from compounds of formula (B), where M represents a suitable metal or metalloid derivative (for example a boronic acid or ester, a trialkyltin group, a suitably substituted silyl group, a zinc derivative or a magnesium halide) by reaction with a compound A-LG in which LG represents a leaving group such as a halogen atom or sulphonate (see reaction scheme 6 below).

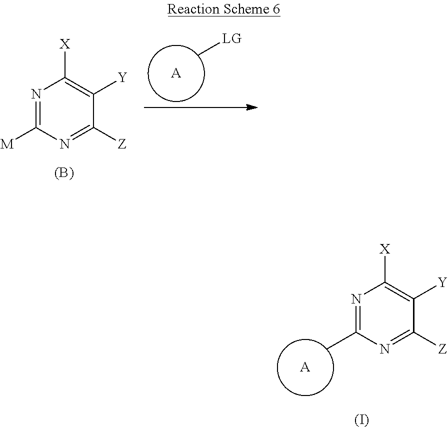

For example, a compound of formula (I) may be prepared from a compound of formula (B) in which M is a boronic acid group by reaction with a compound A-LG in the presence of a metal catalyst (for example a palladium derivative, such as Pd₂(dba)₃), optionally with a suitable ligand (for example a phosphine, such as X-Phos) and a base (for example an inorganic base, such as potassium phosphate or caesium fluoride) in a suitable solvent.

Further compounds of formula (B) may be prepared from different compounds of formula (B) using a transmetallation reaction. For example, a compound of formula (B) where M is a boronic acid may be prepared from a compound of formula (B) where M is a magnesium halide by reaction with a trialkylboronate, followed by hydrolysis (for example under acidic conditions).

Alternatively compounds of formula (B) may be prepared from compounds of formula (A) as shown schematically in reaction scheme 7.

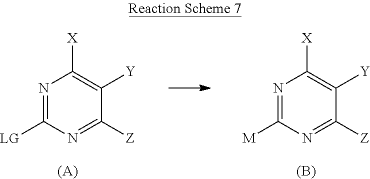

For example, a compound of formula (B) where M is a boronate ester or a trialkylstannane may be prepared from a compound of formula (A) by treating it with a suitable M-containing reagent (for example pinacolborane, bispinacolatodiboron, hexa-alkyldi-tin) in the presence of a metal catalyst (for example a palladium species, such as bis(diphenylphosphine)palladium dichloride) in a suitable solvent (for example an ether, such as dioxane).

Alternatively, a compound of formula (B) where M is a magnesium halide may be prepared from a compound of formula (A) by treatment with a suitable Grignard reagent (for example an isopropylmagnesium halide, such as isopropylmagnesium chloride) in a suitable solvent.

Compounds of formula (A) may be prepared from compounds of formula (C), where LG′ is a second leaving group which may be the same as or different to LG (reaction scheme 8).

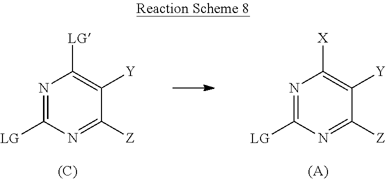

For example, a compound of formula (A) may be prepared from a compound of formula (C) by reaction with a reagent X—H or X⁻ in a suitable solvent (for example an ether solvent, such as tetrahydrofuran). The reagent X⁻ may be generated in situ by addition of a suitable base to X—H, or it may be prepared separately and added as a suitable salt.

In an alternative example a protected version of X—H may be used (for example an oxime may be used as a protected version of hydroxylamine in the synthesis of compounds in which X═ONH₂)—the choice of suitable protecting groups and conditions for removing them will be clear to one skilled in the art.

As an additional example a compound of formula (A) may be prepared from a compound of formula (C) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (C1), which are compounds of formula (C) in which LG=LG′ and is, for example, a halogen atom or a sulphonate, may be prepared from compounds of formula (D)—reaction scheme 9.

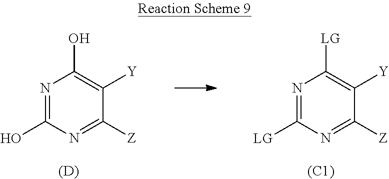

For example, a compound of formula (C1) in which LG is a halogen atom may be prepared from a compound of formula (D) by treatment with a suitable reagent (for example a phosphoryl halide, such as phosphorus oxychloride) and optionally a suitable base (for example an organic base, such as N,N-diethylaniline).

Compounds of formula (C2), that is compounds of formula (C) in which LG and LG′ are different, may be prepared from compounds of formula (E) in which B* represents a suitable precursor group to LG′ (reaction scheme 10).

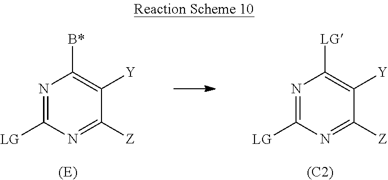

For example, a compound of formula (C2) in which LG′ is a sulphone may be prepared from a compound of formula (E1), which is a compound of formula (E) in which B* is a thioether group, by reaction with a suitable oxidising agent, for example a peracid, such as meta-chloroperbenzoic acid (reaction scheme 11).

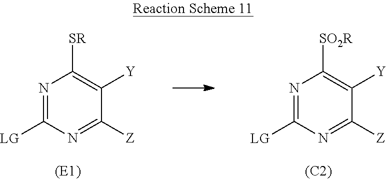

Compounds of formula (E) may be prepared from compounds of formula (C1) by treatment with a reagent B*-H or B*⁻.

For example, as shown in reaction scheme 12, a compound of formula (E1) may be prepared from a compound of formula (C1) by reaction with a thiol or thiolate anion, optionally in the presence of a suitable base, in a suitable solvent.

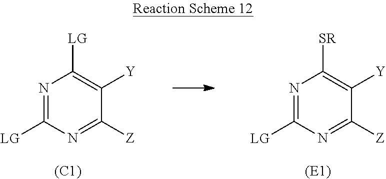

Compounds of formula (D) may be prepared from compounds of formula (F) by reaction with a suitable source of electropositive Y (shown below in reaction scheme 13).

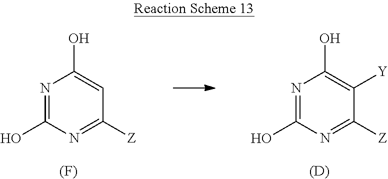

For example a compound of formula (D) in which Y is a halogen may be prepared from a compound of formula (F) by reaction with an N-halosuccinimide (for example N-chlorosuccinimide).

Compounds of formula (F) are well known in the literature, or can be made from compounds known in the literature by standard methods.

Compounds of formula (I) may also be prepared from compounds of formula (G) as shown in reaction scheme 14 below.

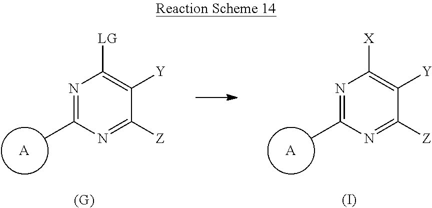

For example, a compound of formula (I) may be prepared from a compound of formula (G) by reaction with a reagent X—H or X⁻ in a suitable solvent (for example methanol or water). The reagent X⁻ may be generated in situ by addition of a suitable base to X—H, or it may be prepared separately and added as a suitable salt.

In an alternative example a protected version of X—H may be used (for example an oxime may be used as a protected version of hydroxylamine in the synthesis of compounds in which X═ONH₂)—the choice of suitable protecting groups and conditions for removing them will be clear to one skilled in the art.

In a further example a compound of formula (I) may be prepared from a compound of formula (G) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (G) may be prepared from compounds of formula (H) (reaction scheme 15 below).

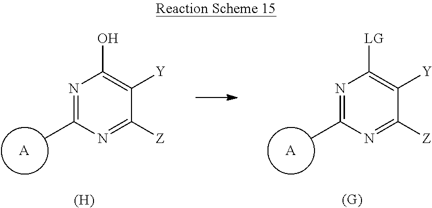

For example, a compound of formula (G) in which LG is a halogen atom may be prepared from a compound of formula (H) by treatment with a suitable reagent (for example a phosphoryl halide, such as phosphorus oxychloride) and optionally a suitable base (for example an organic base, such as N,N-diethylaniline).

Compounds of formula (H) may be prepared from compounds of formula (I) by reaction with a suitable source of electropositive Y (reaction scheme 16 below).

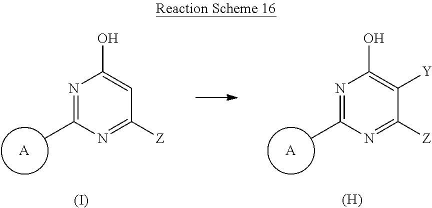

For example a compound of formula (H) in which Y is a halogen may be prepared from a compound of formula (I) by reaction with an N-halosuccinimide (for example N-chlorosuccinimide) in a suitable solvent (for example dimethylformamide), or with a metal hypohalite (for example sodium hypochlorite) in a suitable solvent (for example acidic water).

Compounds of formula (I) may be prepared from amidines of formula (J) by reaction with a suitable ketoester of formula (K), see reaction scheme 17.

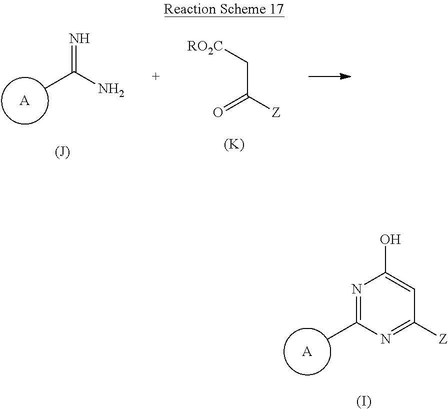

For example, as shown in reaction scheme 18 below, a compound of formula (i1) (which is a compound of formula (i) in which Z is a carboxylic acid or ester) may be prepared by the reaction of an amidine of formula (J) with an oxaloacetate diester of formula (K1) (which is a compound of formula (K) in which Z═CO₂R), optionally in the presence of a suitable base (for example an inorganic base, such as sodium hydroxide), in a suitable solvent (for example water). The diester (K1) may also be used in the form of a salt (for example the sodium salt).

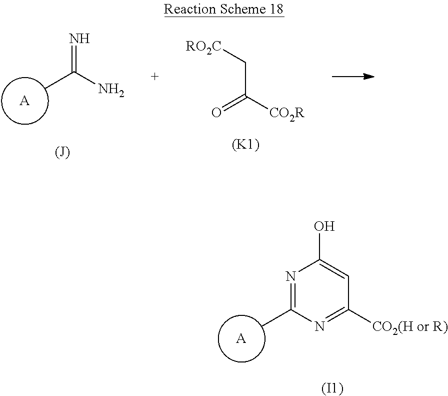

As a further example (reaction scheme 19) a compound of formula (i2) (which is a compound of formula (i) in which Z is an acetal group) may be prepared by the condensation of an amidine of formula (J) with a ketoester of formula (K2) (which is a compound of formula (K) in which Z═CH(OR)₂) in the presence of a base (for example an alkoxide base, such as sodium methoxide) in a suitable solvent (for example an alcohol, such as methanol).

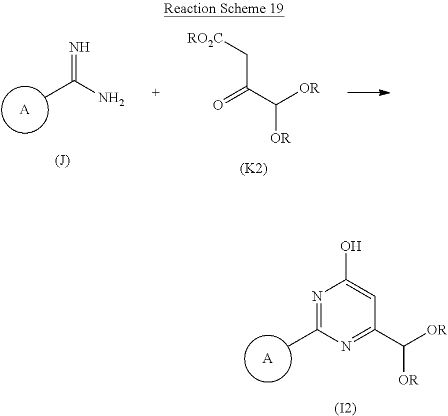

Amidines (J) and diesters (K) are known in the literature or may be easily prepared using standard methods available in the literature.

Compounds of formula (H) may be prepared from amidines of formula (J) by reaction with a suitable ketoester of formula (L) (reaction scheme 20).

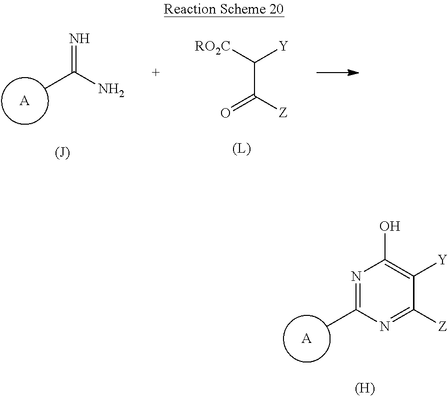

For example (see reaction scheme 21) a compound of formula (H1) (which is a compound of formula (H) in which Z is a carboxylic acid or ester) may be prepared by the reaction of an amidine of formula (J) with an oxaloacetate diester of formula (L1) (which is a compound of formula (L) in which Z═CO₂R), optionally in the presence of a suitable base (for example an inorganic base, such as sodium hydroxide), in a suitable solvent (for example water). The diester (L1) may also be used in the form of a salt (for example the sodium salt).

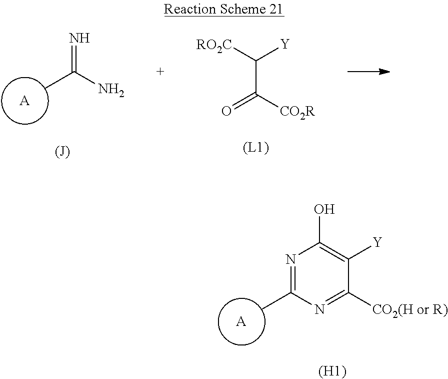

Compounds of formula (L) are known in the literature, or may be prepared from compounds of formula (K) by methods well known in the literature.

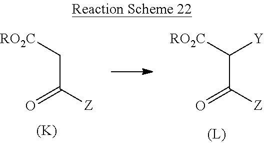

Compounds of formula (H) may be prepared by the condensation of amidines of formula (J) with substituted acetate esters of formula (M) and esters of formula (N) in the presence of a base (for example an alkoxide base, such as sodium ethoxide) in a suitable solvent (for example an alcohol, such as ethanol)—shown schematically below in reaction scheme 23.

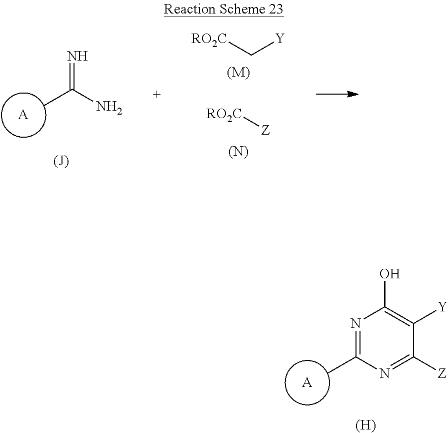

For example a compound of formula (H1) may be prepared by the reaction of an amidine of formula (J) with an acetate ester of formula (M) and a diethyl oxalate of formula (N1) (which is a compound of formula (N) in which Z is a carboxylate ester).

Compounds of formulae (M) and (N) are well known in the literature.

Compounds of formula (I) may additionally be prepared by reaction of compounds of formula (O) with a suitable source of electrophilic Y (reaction scheme 24).

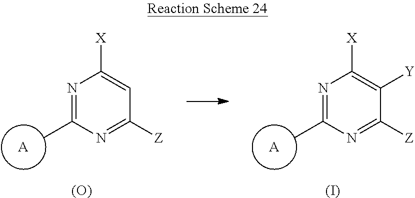

For example a compound of formula (I) in which Y is a halogen may be prepared from a compound of formula (O) by reaction with an N-halosuccinimide (for example N-chlorosuccinimide) in a suitable solvent (for example dimethylformamide).

Compounds of formula (O) may also be prepared from compounds of formula (P) (see reaction scheme 25), in which LG represents a leaving group (for example a halogen, sulphonate or sulphone).

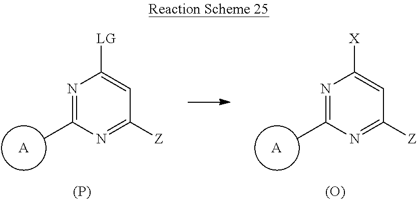

For example, a compound of formula (O) may be prepared from a compound of formula (P) by reaction with a reagent X—H or X⁻ in a suitable solvent (for example an ether solvent, such as tetrahydrofuran). The reagent X⁻ may be generated in situ by addition of a suitable base to X—H, or it may be prepared separately and added as a suitable salt.

In an alternative example a protected version of X—H may be used (for example an oxime may be used as a protected version of hydroxylamine in the synthesis of compounds in which X═ONH₂)—the choice of suitable protecting groups and conditions for removing them will be clear to one skilled in the art.

In a further example a compound of formula (O) may be prepared from a compound of formula (P) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (P) may be prepared from compounds of formula (I) as shown below.

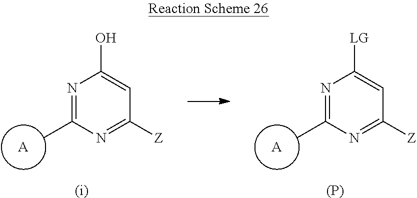

For example, a compound of formula (P) in which LG is a halogen atom may be prepared from a compound of formula (i) by treatment with a suitable reagent (for example a phosphoryl halide, such as phosphorus oxychloride) and optionally a suitable base (for example an organic base, such as N,N-diethylaniline).

Compounds of formula (I) may also be prepared from compounds of formula (Q) (see reaction scheme 27), in which D represents a suitable cyclisation precursor, by reactions in which ring A is formed. Examples of suitable cyclisation precursors include groups containing carbon-carbon and carbon-heteroatom double or triple bonds, such as oximes or alkynes.

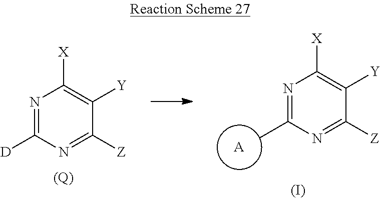

For example (as shown in reaction scheme 28) a compound of formula (I) in which ring A is a 5-membered ring may be formed from a compound of formula (Q1) (which is a compound of formula Q in which D is an alkyne) by reaction with a suitable 1,3-dipolar species R in which one or more of E, F and G are heteroatoms, such as an azide or nitrile oxide.

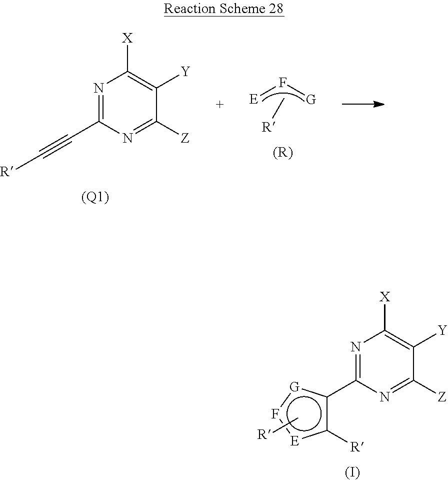

In a further example (see reaction scheme 29) a compound of formula (I) in which ring A is an isoxazole may be prepared by reaction of a compound of formula (Q2) (which is a compound of formula Q in which D is an aldoxime) first with an oxidising reagent (for example an N-halosuccinimide) in the presence of a base, and then with an alkyne of formula (S) under suitable conditions.

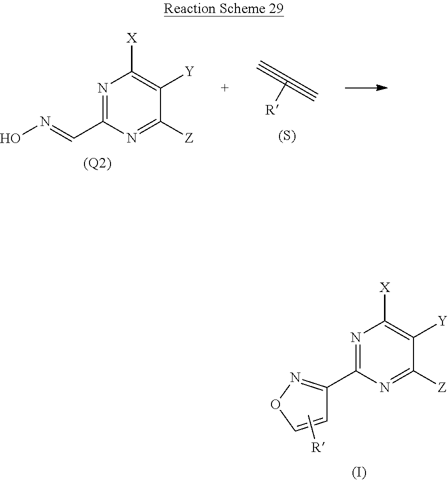

In another example (see reaction scheme 30) a compound of formula (I) may be formed by the reaction of a compound of formula (Q3) (which is a compound of formula (Q) in which D is a ketone containing a leaving group, such as a halogen atom or a sulphonate) with an acid derivative of formula (T).

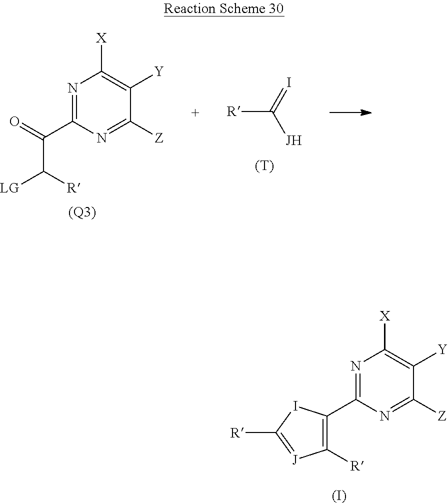

For example (see reaction scheme 31) a compound of formula (I) in which ring A is an imidazole may be prepared by the reaction of a compound of formula (Q3) with an amidine of formula (T1) (which is a compound of formula (T) in which I and J are both N—H groups).

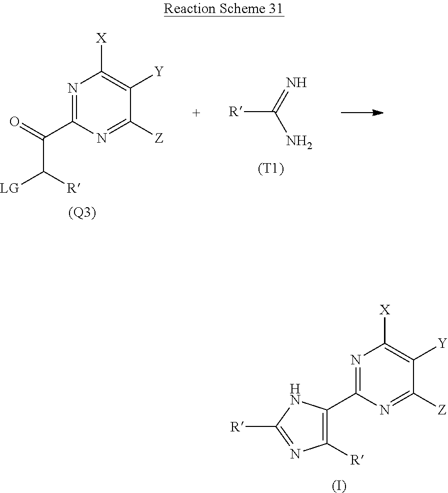

Compounds of formulae (R), (S) and (T) are known or can be made easily using methods that are well known in the literature.

Compounds of formula (Q1) may be prepared from compounds of formula (A) by a Sonogashira-type reaction (see reaction scheme 32) with an alkyne (S1) (which is an alkyne of formula (S) in which at least one substituent is a hydrogen atom).

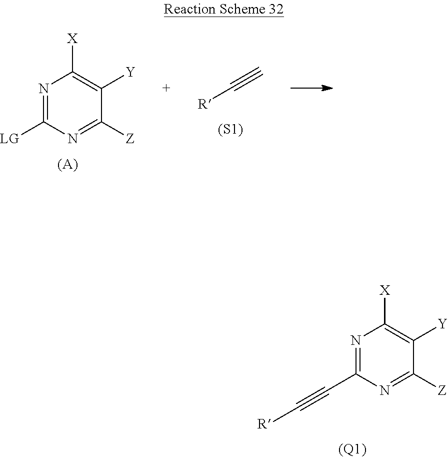

Compounds of formula (Q2) may be prepared from compounds of formula (U) (see reaction scheme 33) by reaction with hydroxylamine or a salt of hydroxylamine, in the presence of a base (for example an organic base, such as triethylamine).

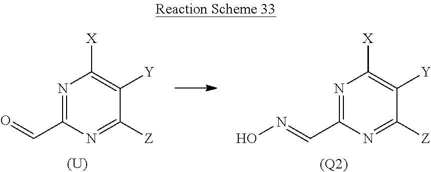

Compounds of formula (U) may be prepared by the reaction of compounds of formula (B1) (which are compounds of formula (B) in which M is MgHal) with a formamide such as dimethylformamide (reaction scheme 34).

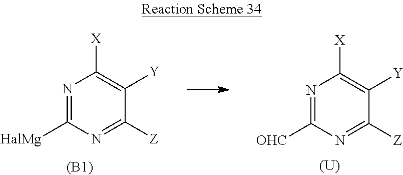

Alternatively (see reaction scheme 35), compounds of formula (U) may be prepared from compounds of formula (V) by oxidative cleavage of the double bond, for example by treatment with ozone.

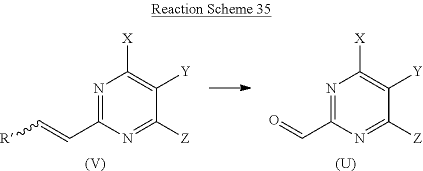

Compounds of formula (Q3) may be prepared from compounds of formula (W) (see reaction scheme 36) by reaction with a suitable oxidising reagent (for example an N-halosuccinimide in the case that LG is a halogen atom), optionally in the presence of a suitable base.

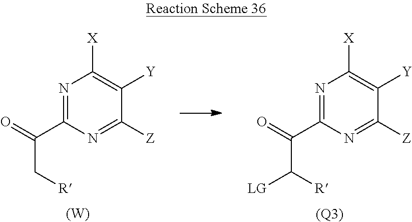

Compounds of formula (W) may be prepared from compounds of formula (X) by oxidative cleavage of the double bond, for example by treatment with ozone (reaction scheme 37).

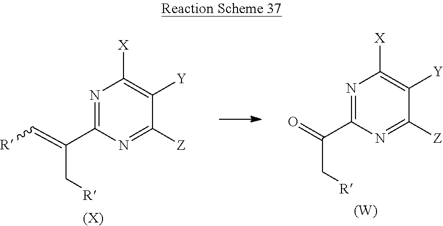

Compounds of formulae (V) or (X) may be made from compounds of formula (A) using one of many reactions well known in the literature, as shown schematically below.

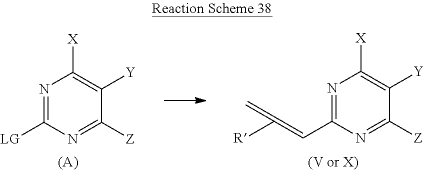

For example (see reaction scheme 39) a compound of formula (V) or (X) may be prepared from a compound of formula (A) by reaction with an alkene of formula (Y) under conditions of the Heck reaction.

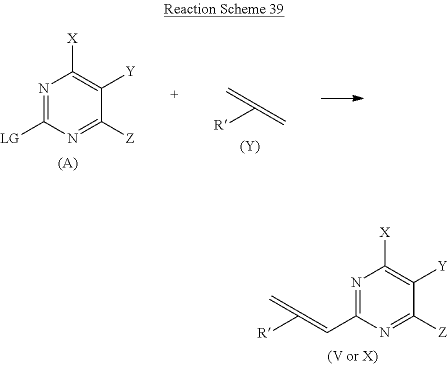

In a further example (see reaction scheme 40) a compound of formula (V) or (X) may be prepared from a compound of formula (A) by reaction with a compound of formula (Z), in which M represents a metal or metalloid species (for example a boronic acid or trialkyltin group) under the appropriate reaction conditions for each specific metal (for example using conditions for the Suzuki-Miyaura reaction when M is a boronic acid or boronate ester).

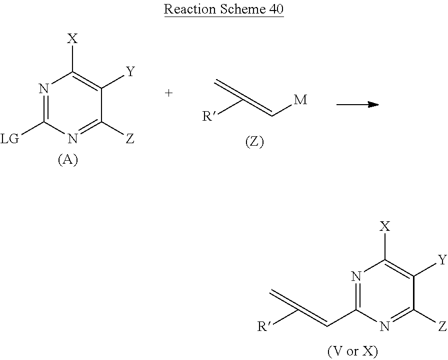

Species of formula (Z) are known, or may be made from compounds that are known using standard methods well known in the literature.

Alternatively compounds of formula (U) or (W) may be prepared from compounds of formula (AA) by treatment with a suitable oxidising agent, for example sodium periodate (see reaction scheme 41).

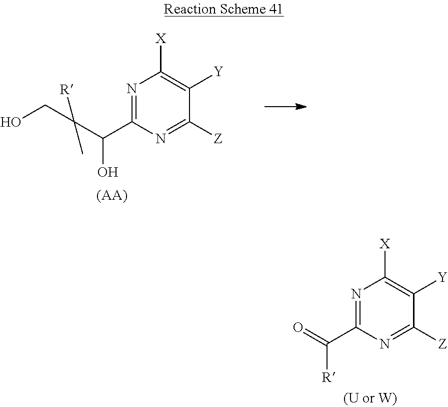

Compounds of formula (AA) may be prepared from compounds of formula (V) or (X) (see reaction scheme 42) by reaction with a suitable dihydroxylation reagent (for example an osmium species such as osmium tetroxide).

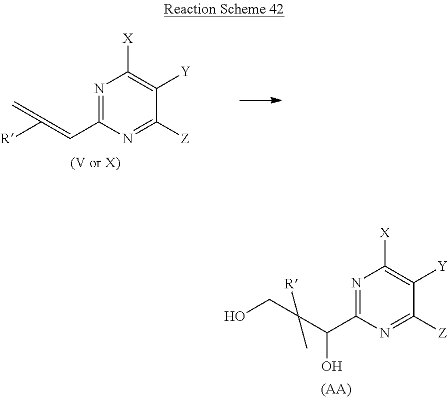

Compounds of formula (I) may be prepared by the condensation of compounds of formula (AB), in which K is a carboxylic acid derivative, such as an amide or thioamide, with suitably functionalised carbonyl containing compounds (reaction scheme 43 below).

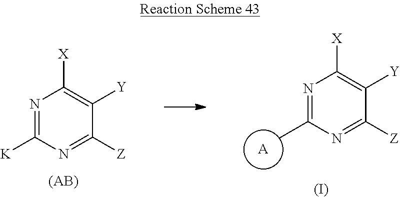

For example (as shown in reaction scheme 44) a compound of formula (I) in which ring A is a thiazole may be prepared from a thioamide of formula (AB1) (which is a compound of formula (AB) in which K is a thioamide group) by reaction with a compound of formula (AC), where LG is a leaving group, for example a halogen or sulphonate.

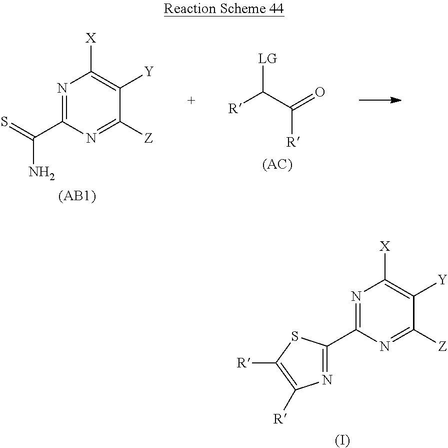

Compounds of formula (AC) are known compounds or may be prepared using methods well known in the literature.

Compounds of formula (AB) may be prepared from compounds of formula (B) by reaction with an electrophilic source of the group K—see reaction scheme 45 below.

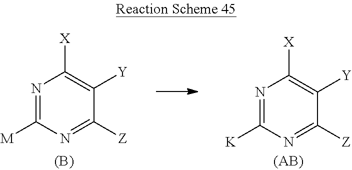

For example, a compound of formula (AB2) (which is a compound of formula (AB) in which K is a carboxylic acid) may be prepared from a compound of formula (B1) by reaction with carbon dioxide in a suitable solvent (reaction scheme 46).

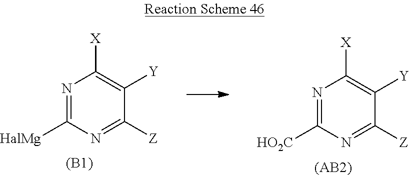

Compounds of formula (AB) may be prepared from other compounds of formula (AB) using transformations that are well known in the literature. For example (see reaction scheme 47), a compound of formula (AB1) may be prepared from a compound of formula (AB2) by first forming an amide of formula (AB3) (which is a compound of formula (AB) in which K is an amide group) under known conditions, followed by reaction with a suitable thionating reagent, such as phosphorus pentasulphide or Lawesson's reagent.

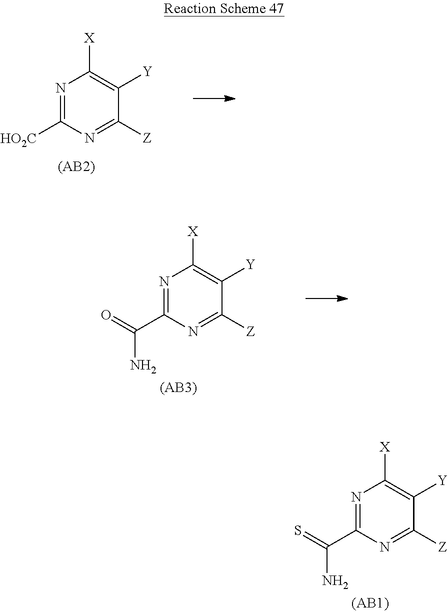

Compounds of formula (O1) (which are compounds of formula (O) in which X═NH₂) may be prepared (see reaction scheme 48) by the reaction of amidines of formula (J) with cyanoketones of formula (AD) in the presence of a base (for example an alkoxide base, such as sodium methoxide) in a suitable solvent (for example an alcohol, such as ethanol)

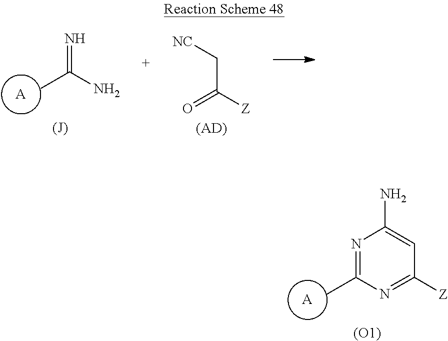

For example (reaction scheme 49) a compound of formula (O2) (which is a compound of formula (O) in which X═NH₂ and Z═CO₂R) may be prepared by the reaction of an amidine of formula (J) with a cyanopyruvate ester of formula (AD1) (which is a compound of formula (AD) in which Z═CO₂R). In one example the compound of formula (AD1) may be reacted first with an alkylating agent (for example a methylating agent, such as dimethyl sulphate) in the presence of a base (for example an inorganic base, such as sodium bicarbonate) to form an enol ether, which is then reacted with amidine (J) in the presence of a base (for example an alkoxide base, such as sodium methoxide).

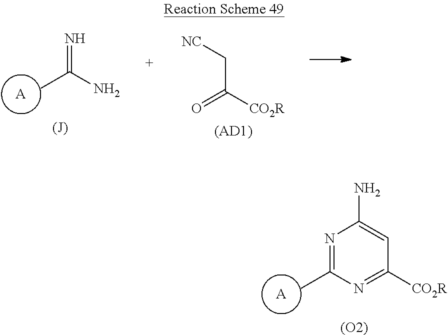

Compounds of formula (AD) are known or may be prepared using known methods.

Compounds of formula (I) in which X═NH₂ may be prepared (see reaction scheme 50) by the condensation of amidines of formula (J) with substituted acetonitriles of formula (AE) and esters of formula (N) in the presence of a base (for example an alkoxide base, such as sodium ethoxide) in a suitable solvent (for example an alcohol, such as ethanol).

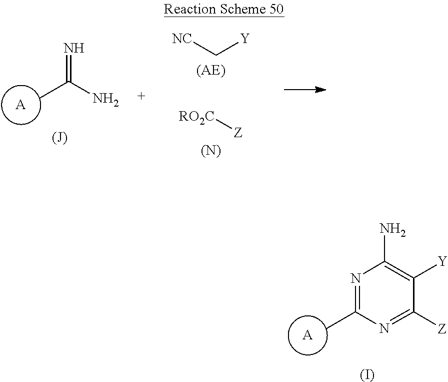

For example, a compound of formula (I) in which X═NH₂ and Z═CO₂R may be prepared by the reaction of an amidine of formula (J) with a substituted acetonitrile of formula (AE) and an oxalate diester of formula (N1).

Compounds of formula (AE) are known in the literature.

Compounds of formula (I) may be prepared from compounds of formula (AF) (see reaction scheme 51), where LG is a suitable leaving group, such as a halogen atom or sulphonate.

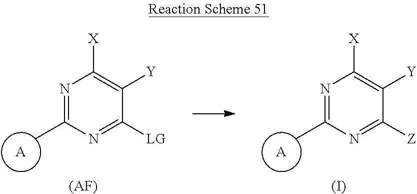

For example (see reaction scheme 52) a compound of formula (I) in which Z═CO₂R may be prepared from a compound of formula (AF) by reaction with an alcohol ROH and carbon monoxide in the presence of a suitable metal catalyst (for example a palladium reagent, such as bis(triphenylphosphine)palladium dichloride) and a suitable base (for example an organic base, such as triethylamine). It may conveniently be conducted under an atmosphere of carbon monoxide gas at atmospheric or raised pressure.

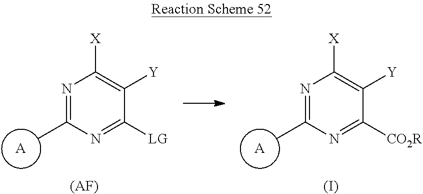

Alternatively (see reaction scheme 53) a compound of formula (I) in which Z=T-(U)ₙ—V may be prepared from a compound of formula (AF) by reaction with a compound of formula AG in the presence of a base (for example sodium hydride) in a suitable solvent (for example an ether, such as tetrahydrofuran).

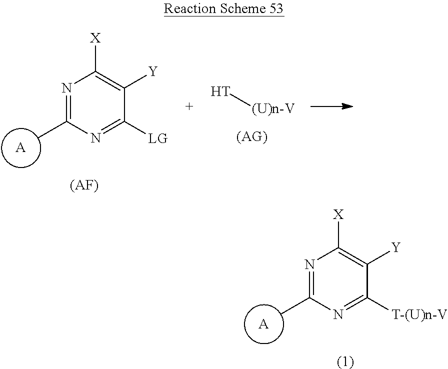

Compounds of formula (AG) are known compounds or may be prepared from known compounds using methods that are well known in the literature.

Compounds of formula (AF) may be prepared from compounds of formula (AH) by reaction with a suitable reagent (reaction scheme 54).

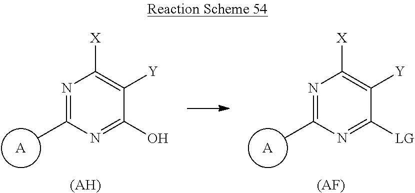

For example a compound of formula (AF) in which LG is a halogen atom (for example a chlorine atom) may be prepared from a compound of formula (AH) by reaction with the corresponding phosphoryl halide (for example phosphorus oxychloride), optionally in the presence of a suitable base (for example an organic base, such as N,N-diethylaniline).

Alternatively (see reaction scheme 55) compounds of formula (AF) may be prepared from compounds of formula (A1) in which LG and LG′ may be the same or different and are leaving groups such as a halogen atoms or sulphonates.

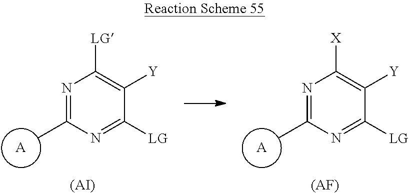

For example, a compound of formula (AF) may be prepared from a compound of formula (A1) by reaction with a reagent X—H or X⁻ in a suitable solvent (for example an ether solvent, such as tetrahydrofuran). The reagent X⁻ may be generated in situ by addition of a suitable base to X—H, or it may be prepared separately and added as a suitable salt.

In another example, a protected version of X—H may be used (for example an oxime may be used as a protected version of hydroxylamine in the synthesis of compounds in which X═ONH₂)—the choice of suitable protecting groups and conditions for removing them will be clear to one skilled in the art.

As a further example, a compound of formula (AF) may be prepared from a compound of formula (A1) by treatment with a reagent X—H in the presence of a suitable catalyst (for example a metal catalyst, such as a palladium source) and optionally a suitable ligand (for example a phosphine ligand, such as Josiphos) in a suitable solvent.

Compounds of formula (AH1) (which are compounds of formula (AH) in which X═NH₂) may be prepared by the reaction of amidines of formula (J) with cyanoacetate esters of formula (AJ) in the presence of a suitable base (for example an organic base, such as DBU) in a suitable solvent (for example a polar aprotic solvent, such as dimethylformamide), see reaction scheme 56 below.

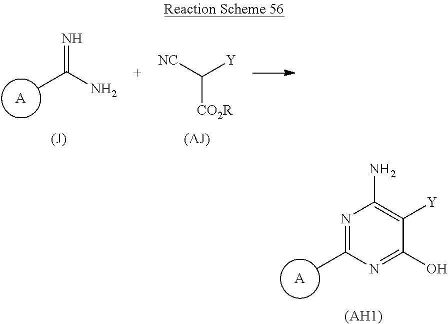

Compounds of formula (AJ) are known or may be prepared using standard methods that are well known in the literature.

Compounds of formula (AI1) (which are compounds of formula (AI) in which LG=LG′) may be prepared from compounds of formula (AK) by reaction with a suitable reagent, for example a phosphoryl halide or sulphonyl anhydride (shown schematically in reaction scheme 57 below).

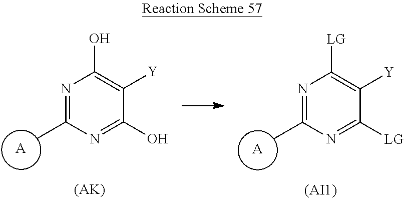

For example, a compound of formula (AI1) in which LG and LG′ are halogen atoms may be prepared by reaction of a compound of formula (AK) with a halogenating agent (for example a phosphoryl halide, such as phosphorus oxychloride), optionally in the presence of a base (for example an organic base, such as N,N-diethylaniline). Compounds of formula (AK) may be prepared by the reaction of amidines of formula (J) with malonyl diesters of formula (AL) (reaction scheme 58 below) in the presence of a suitable base (for example an organic base, such as DBU) in a suitable solvent (for example a polar aprotic solvent, such as N-methylpyrrolidone).

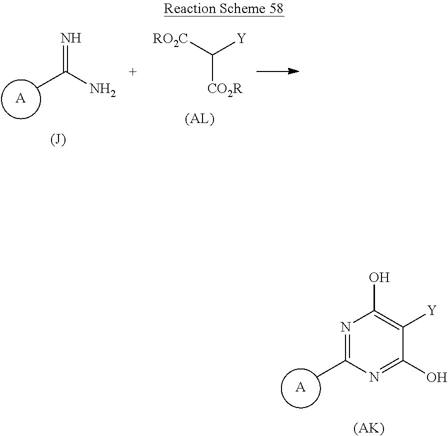

Diesters of formula (AL) are known in the literature or may be prepared by methods known in the literature.

Alternatively, compounds of formula (AK) may be prepared by reaction of compounds of formula (AM) with a suitable source of electropositive Y (reaction scheme 59).

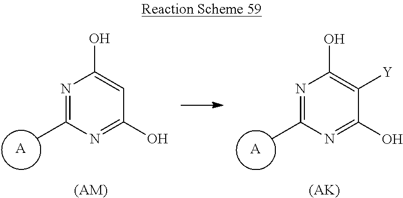

For example a compound of formula (AK) in which Y is a halogen may be prepared from a compound of formula (AM) by reaction with a halogenating agent (for example an N-halosuccinimide, such as N-chlorosuccinimide, or an elemental halogen, such as bromine).

Compounds of formula (AM) may be prepared from amidines of formula (J) and malonyl diesters (reaction scheme 60).

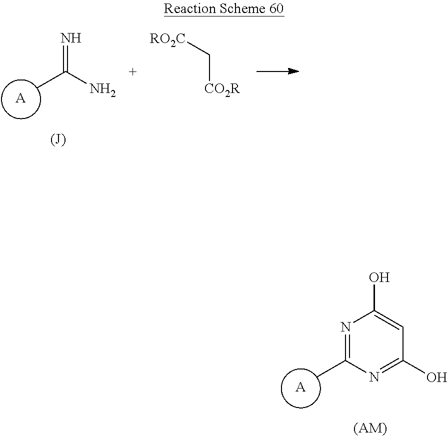

Malonyl diesters are well known in the literature.

Compounds of formula (I) in which Z=T-(U)ₙ—V may be prepared from compounds of formula (AH) or (AN) by reaction with compounds of formula (AO) (see reaction scheme 61 below) in which LG is a suitable leaving group, such as a halogen or sulphonate group, in the presence of a base (for example sodium hydride) in a suitable solvent (for example an ether, such as tetrahydrofuran).

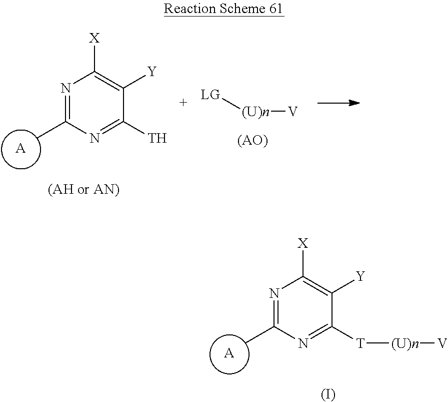

Compounds of formula (AO) are known or may be prepared from known compounds, for example from compounds of formula (AG), using methods that are well known in the literature.

Compounds of formula (AN) may be prepared from compounds of formula (AH) by treatment under controlled conditions with a suitable thionating agent (for example phosphorus pentasulphide or Lawesson's reagent)—see reaction scheme 62.

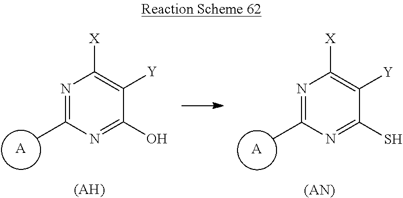

Compounds of formula (AH) may be prepared by reaction of compounds of formula (AP) with a suitable source of electropositive Y (reaction scheme 63).

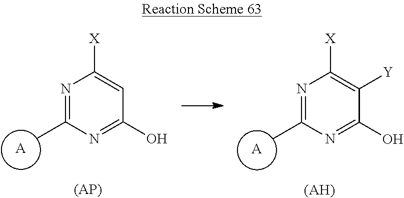

For example a compound of formula (AH) in which Y is a halogen may be prepared from a compound of formula (AP) by reaction with a halogenating agent (for example an N-halosuccinimide, such as N-chlorosuccinimide, or an elemental halogen, such as bromine).

Compounds of formula (AP1) (which are compounds of formula (AP) in which X is NH₂) may be prepared by condensation of amidines of formula (J) with cyanoacetate esters in the presence of a base (for example an alkoxide base, such as sodium methoxide) in a solvent (for example an alcohol, such as methanol)—reaction scheme 64.

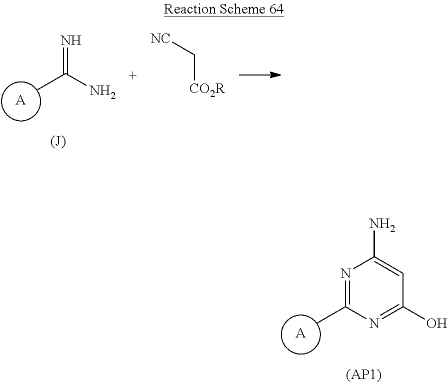

Cyanoacetate esters are well known in the literature.

Compounds of formula (I) in which X is OR may be prepared by the alkylation of compounds of formula (H) (reaction scheme 65). Suitable alkylating agents include alkyl halides (for example methyl iodide) or sulphonates in the presence of a base, or diazocompounds (for example diazomethane or trimethylsilyldiazomethane).

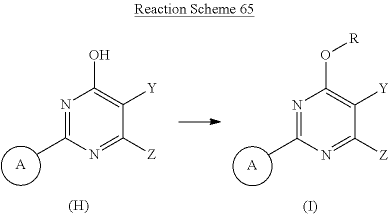

Compounds of formula (I) in which X is NRCH(R′)CONHR′ may be prepared by the reaction of compounds of formula (H) with an amine of formula RNH₂, an aldehyde of formula R′CHO and an isonitrile of formula R′NC (see reaction scheme 66 below).

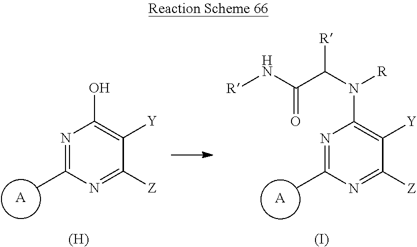

Compounds of formula (I) in which m is 0 and n is 2, may be prepared (reaction scheme 67) by the reaction of compounds of formula (AQ) with a reagent that can functionalise the double bond.

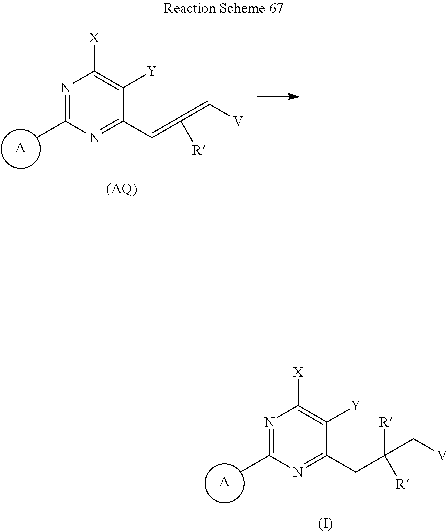

For example, a compound of formula (I) in which the groups R′ are hydrogen may be prepared from a compound of formula (AQ) by reaction with a suitable reducing agent (for example hydrogen gas in the presence of a metal catalyst, such as palladium supported on carbon) (see reaction scheme 68, below).

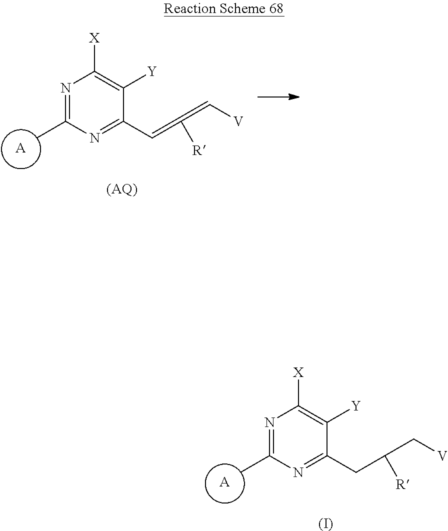

As a further example (reaction scheme 69), a compound of formula (I) in which the groups R′ represent vicinal hydroxyl groups may be prepared from a compound of formula (AQ) by reaction with a dihydroxylation reagent (for example osmium tetroxide).

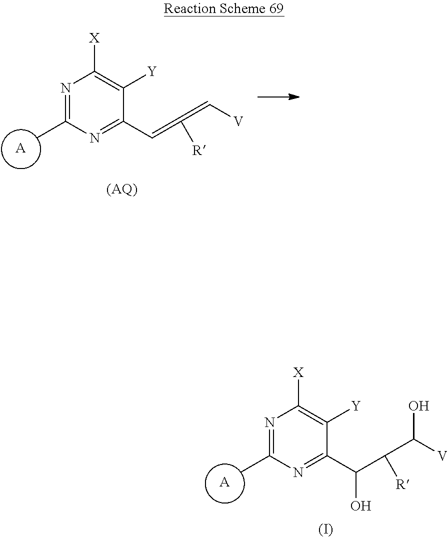

Compounds of formula (AQ) may be prepared by the Heck reaction of compounds of formula (AF) with compounds of formula (AR) (reaction scheme 70) in the presence of a suitable metal catalyst (for example a palladium species, such as palladium acetate) and a base (for example an organic base, such as triethylamine), in a suitable solvent.

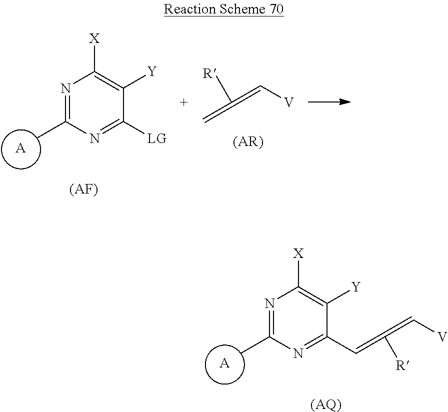

Alternatively, compounds of formula (AQ) may be prepared by the reaction of compounds of formula (I) in which Z is CHO under the conditions of a suitable olefination reaction, for example a Wittig, Horner-Emmons or Peterson reaction (reaction scheme 71).

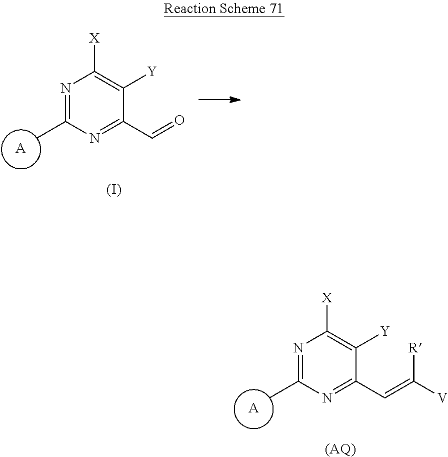

Compounds of formula (I) may be prepared from different compounds of formula (I) by the conversion of any of the substituents X, Y and Z and the ring A into a different group X, Y, Z or A using techniques known in the literature.

For example, one substituent on ring A may be transformed into another substituent using methods well known in the literature. One such example is the conversion of a ring hydrogen into a halogen using a suitable halogenation agent (for example an N-halosuccinimide, such as N-chlorosuccinimide).

A further example is the conversion of a compound in which Y is a halogen or sulphonate (for example a bromine or chlorine atom) into a compound in which Y is a carbon-based group, for example an alkyl or alkenyl group or an optionally substituted phenyl or heteroaryl ring. Such a transformation may be carried out by reaction with a metal or metalloid derivative of the alkyl or alkenyl group or phenyl or heteroaryl ring (for example a boronic acid or boronate ester) in the presence of a base (for example an inorganic base, such as potassium phosphate or caesium fluoride, or an organic base, such as triethylamine), a metal source (for example a palladium source, such as Pd₂(dba)₃) and, optionally, a ligand for the metal (for example a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (for example a palladium/phosphine complex, such as palladium tetrakistriphenylphosphine, bis(triphenylphosphine)palladium dichloride or [1,1-bis(diphenylphosphino)ferrocene]palladium dichloride).

Yet another example is the preparation of a compound of formula (I) in which X is NRCOR′ from a compound of formula (I) in which X is NHR by reaction with an acylating agent (for example an acyl chloride) in the presence of a base.

In a further example (reaction scheme 72) a compound of formula (I) in which V is a carboxylic acid may be prepared from a compound of formula (I) in which V is a carboxylate ester, by hydrolysis under basic or acidic conditions, for example by treatment with aqueous sodium hydroxide. Alternatively this transformation may be achieved by treatment of the ester with a nucleophile, for example an alkyl thiolate, in a suitable solvent.

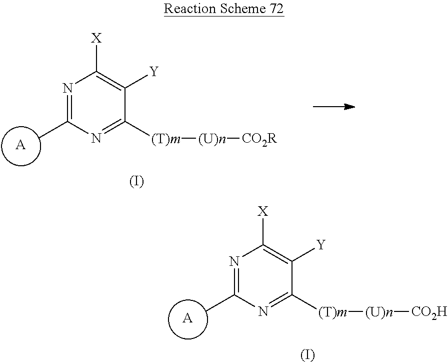

A compound of formula (I) in which V is a carboxylate ester may be prepared directly from a compound of formula (I) in which V is a carboxylic acid by esterification under standard conditions, for example by treatment with an alcohol ROH and an acid catalyst. The acid catalyst may conveniently be generated in situ by addition of a compound that reacts with the alcohol to generate an acid (for example, thionyl chloride or acetyl chloride). Alternatively, this transformation may be achieved by first preparing an activated derivative of the acid group, for example an acyl halide, followed by reaction with an alcohol.

Other derivatives of the acid group in compounds of formula (I) in which V is a carboxylic acid may be prepared by standard methods found in the literature. For example a compound of formula (I) in which V is an amide group may be prepared from a compound of formula (I) in which V is a carboxylic acid by treatment with a suitable coupling reagent (for example a carbodiimide, such as dicyclohexylcarbodiimide) and an amine R′₂NH, optionally with a additive (for example dimethylaminopyridine), in a suitable solvent (for example dimethylformamide)—see reaction scheme 73. Alternatively, this transformation may be performed by first preparing an activated derivative of the carboxylic acid group (for example an acyl halide, such as an acid chloride), and then treating the activated derivative with an amine RR′NH.

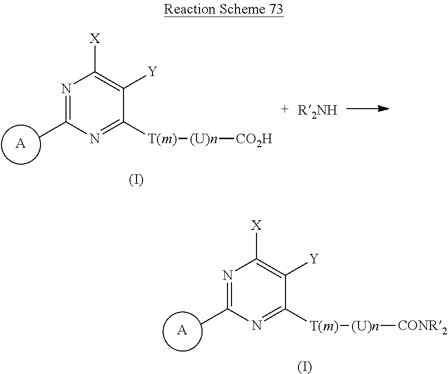

A compound of formula (I) in which Z is CO₂R may be prepared from a compound of formula (I) in which Z is CH(OR)₂ by treatment with a suitable oxidising agent (for example N-bromosuccinimide or a persulphate salt), optionally in the presence of an acid (for example sulphuric acid) (reaction scheme 74).

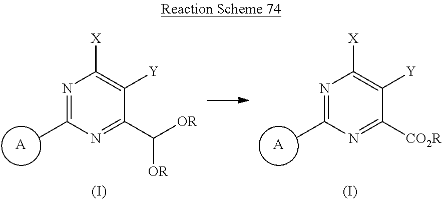

A compound of formula (I) in which V is CHO may be prepared (see reaction scheme 75) from a compound of formula (I) in which V is CO₂R by treatment with a suitable reducing agent (for example a hydride reducing agent, such as DIBAL-H).

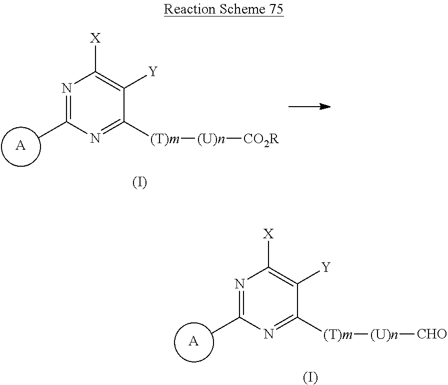

A compound of formula (I) in which V is CO₂H may be prepared from a compound of formula (I) (see reaction scheme 76) in which V is CHO by treatment with a suitable oxidising agent (for example sodium hypochlorite).

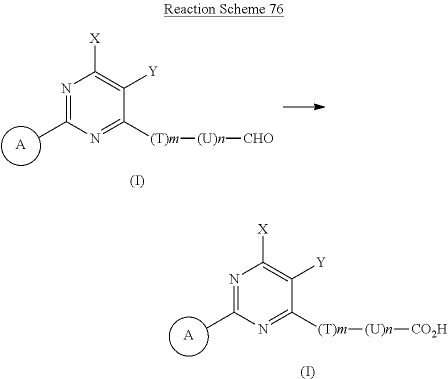

A compound of formula (I) in which V is CHO may be prepared by the removal of a suitable aldehyde protecting group (APG) from a compound of formula (I) in which V is a protected aldehyde (reaction scheme 77). Some examples of suitable protecting groups include acetals, thioacetals and hydrazones.

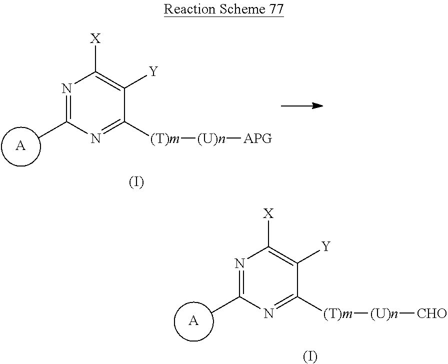

For example (reaction scheme 78), a compound of formula (I) in which V is CHO may be prepared from a compound of formula (I) in which the aldehyde protecting group is an acetal (for example a dialkyl acetal, such as a diethyl acetal) by treatment with acid (for example an inorganic acid, such as aqueous hydrochloric acid).

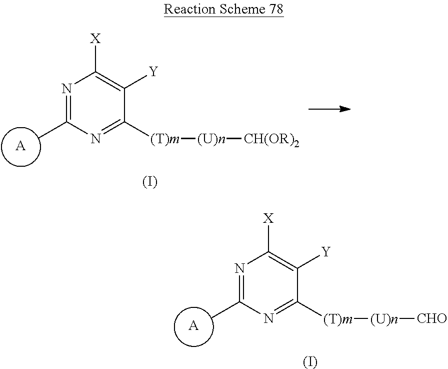

A compound of formula (I) in which V is CHO may also be prepared from a compound of formula (I) in which V is CH₂OH by oxidation under standard conditions (for example using the Swern reaction)—see reaction scheme 79 below.

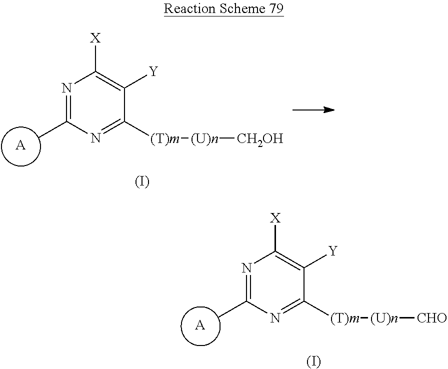

A compound of formula (I) in which V is CHO may be converted into a derivative (for example an acetal or dithioacetal) of formula (I) under conditions that are known in the literature.

A compound of formula (I) in which V is CH₂OH may be prepared from a compound of formula (I) in which V is CHO (reaction scheme 80) by reaction with a suitable reducing agent (for example a metal hydride reducing agent, such as sodium borohydride).

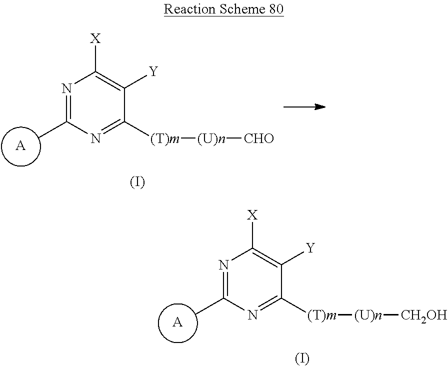

Alternatively, a compound of formula (I) in which V is CH₂OH may be prepared from a compound of formula (I) in which V is CO₂R (see reaction scheme 81) by treatment with a suitable reducing agent (for example a metal hydride reducing agent, such as lithium aluminium hydride).

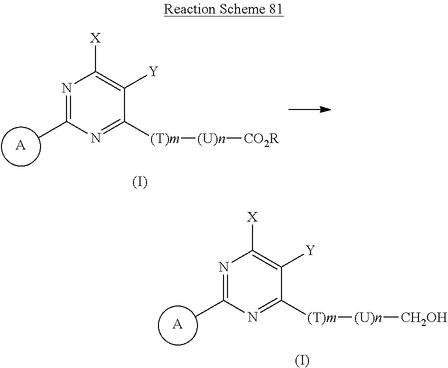

A compound of formula (I) in which V is CH₂OC(O)R′ may be prepared from a compound of formula (I) in which V is CH₂OH under standard conditions (see reaction scheme 82) for example by treatment with an acyl halide in the presence of a base.

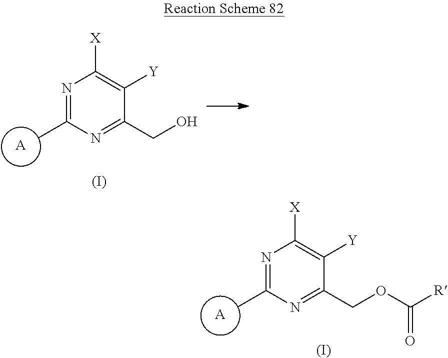

One skilled in the art will understand that transformations of this type may equally well be conducted at different stages of the synthetic route, for example converting one compound of formula (A) into a different compound of formula (A) or one compound of formula (H) into a different compound of formula (H).

A person skilled in the art will understand that in certain instances more than one transformation can be conducted at one time utilising the same reaction conditions. For example (see reaction scheme 83) a compound of formula (I) in which ring A and Y are the same may be prepared from a compound of formula (A2) (which is a compound of formula (A) in which Y is a leaving group such as a halogen atom or a sulphonate, which may be the same or different to LG) by reaction with an excess of a metal or metalloid derivative of ring A, such as a boronic acid, in the presence of a metal catalyst (for example a palladium derivative, such as Pd₂(dba)₃), a ligand (for example a phosphine ligand, such as X-Phos) and a base (for example an inorganic base, such as potassium phosphate) in a suitable solvent (for example a single solvent, such as acetonitrile, or a mixed solvent system, such as a mixture of dimethoxyethane and water).

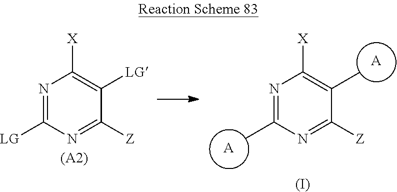

Another example (reaction scheme 84) is the preparation of a compound of formula (I) in which X is NR′₂ and V is CONR′₂ from a compound of formula (G1) (which is a compound of formula (G) in which V is CO₂R) by treatment with an excess of an amine R′₂NH in a suitable solvent.

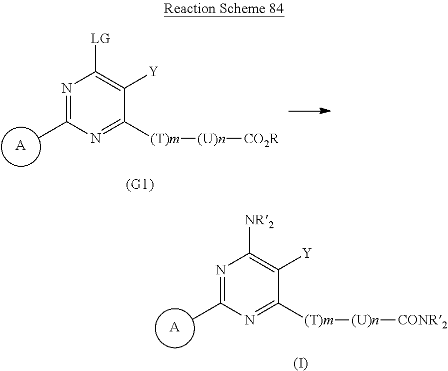

A further example (reaction scheme 85) is the preparation of a compound of formula (H1) from a compound of formula (I2) by oxidation using a reagent that also provides a source of electropositive Y (for example, N-bromosuccinimide for the case in which Y═Br).

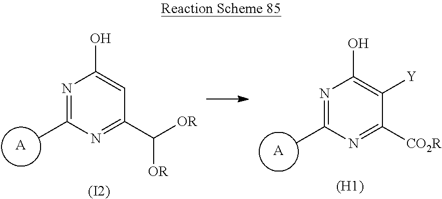

An additional example (reaction scheme 86) is the preparation of a compound of formula (I) in which X is OR and V is CO₂R from a compound of formula (G2) (which is a compound of formula (G) in which V═CO₂H) by treatment with an alcohol ROH and a suitable acid catalyst (for example concentrated sulphuric acid).

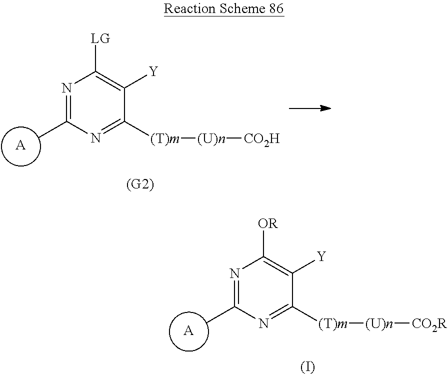

One skilled in the art will realise that it is often possible to alter the order in which the transformations above are conducted or to combine them in alternative ways to prepare a wide range of compounds of formula (I).

One skilled in the art will also realise that some reagents will be incompatible with certain values or combinations of substituents X, Y, Z and A, and additional steps, such as protection and deprotection steps, will be necessary to achieve the desired transformation.

Compounds of formula (I) may be used in unmodified form, i.e. as obtainable from synthesis, but preferably are formulated in any suitable manner using formulation adjuvants, such as carriers, solvents and surface-active substances, for example, as described hereinafter. Thus in a further aspect the invention provides a herbicidal composition comprising a compound of formula (I), in particular a compound of formula (IA) and at least one agriculturally acceptable formulation adjuvant or diluent.

The formulations can be in various physical forms, e.g. in the form of dusting powders, gels, wettable powders, water-dispersible granules, water-dispersible tablets, effervescent pellets, emulsifiable concentrates, microemulsifiable concentrates, suspension concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. The formulations can be in the form of concentrates which are diluted prior to use, although ready-to-use formulations can also be made. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules usually have a diameter of from 0.1 to 500 microns. Typically, they will contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other known polymers. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in 40 CFR 180.910 and 40 CFR180.920.

A large number of surface-active substances may advantageously be used in the formulations, especially in those formulations designed to be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilisers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈₋₂₂ fatty acids, especially the methyl derivatives of C₁₂₋₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Another preferred adjuvant is Adigor® (Syngenta AG) which is a methylated rapeseed oil-based adjuvant.

The application and action of the oil additives can be further improved by combination with surface-active substances, such as non-ionic, anionic or cationic surfactants. Examples of suitable anionic, non-ionic and cationic surfactants are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂₋₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

Herbicidal compositions of the invention generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. Whereas commercial products will preferably be formulated as concentrates, the end user will normally employ dilute formulations.

Examples of preferred formulation types and their typical compositions are given below (% is percent by weight). Wettable powders as described herein are one particularly preferred type of formulation for use in the invention. In other preferred embodiments, in particular where the compound/composition/formulation of the invention is intended for use on turf, granular (inert or fertiliser) formulations as described herein are particularly suitable.

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%
The following Examples further illustrate, but do not limit, the invention.

### Formulation Examples for Herbicides of Formula (I) (%=% by Weight)

F1. Emulsifiable concentrates

| | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon mixture | 85% | 78% | 55% | 16% |
| (C₉-C₁₂) | | | | |

Emulsions of any desired concentration can be obtained from such concentrates by dilution with water.

F2. Solutions

| | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| (C₉-C₁₂) | | | | |

The solutions are suitable for use in the form of microdrops.

F3. Wettable powders

| | a) | b) | c) | d) |
|---|---|---|---|---|
| active ingredient | 5% | 25% | 50% | 80% |
| sodium lignosulfonate | 4% | — | 3% | — |
| sodium lauryl sulphate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

F4. Coated granules

| | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

F5. Coated granules

| | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

F6. Extruder granules

| | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

F7. Dusts

| | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

F8. Suspension concentrates

| | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

Compounds of the invention (as well as mixtures and/or formulations containing the same) find utility as herbicides, and may thus be employed in methods of controlling plant growth. Such methods involve applying to the plants or to the locus thereof an herbicidally effective amount of said compound, or composition comprising the same (or mixture as described hereinafter). The invention thus also relates to a method of inhibiting plant growth which comprises applying to the plants or to the locus thereof a herbicidally effective amount of a compound of formula (I), composition, or mixture of the invention. In particular the invention provides a method of controlling weeds in crops of useful plants, which comprises applying to said weeds or the locus of said weeds, or to said crop of useful plants, a compound of formula I or a composition or mixture containing the same.

The term “locus” as used herein includes not only areas where weeds may already be growing, but also areas where weeds have yet to emerge, and also to areas under cultivation with respect to crops of useful plants. Areas under cultivation include land on which the crop plants are already growing and land intended for cultivation with such crop plants.

A compound, composition, and/or mixture of the invention may be used in a pre-emergence application and/or in a post-emergence application in order to mediate its effect.

Crops of useful plants in which compounds of formula (I), as well as formulations and/or mixtures containing the same, may be used according to the invention include perennial crops, such as citrus fruit, grapevines, nuts, oil palms, olives, pome fruit, stone fruit and rubber, and annual arable crops, such as cereals, for example barley and wheat, cotton, oilseed rape, maize, rice, soy beans, sugar beet, sugar cane, sunflowers, ornamentals and vegetables, especially cereals and maize.

Compounds of formula (I), formulations and/or mixtures containing the same may also be used on turf, pasture, rangeland, rights of way etc. In particular they may be used on golf-courses, lawns, parks, sports-fields, race-courses and the like.

Crops are to be understood as also including those crops which have been rendered tolerant to herbicides or classes of herbicides (e.g. ALS-, GS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink® as well as corn, soybean and cotton that have been engineered to be resistant to Dicamba, phenoxypropionic acids, pyridyloxyacetic acids and/or picolinate auxines.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

The term “weeds” as used herein means any undesired plant, and thus includes not only agronomically important weeds as described below, but also volunteer crop plants.

Compounds of formula (I) may be used against a large number of agronomically important weeds. The weeds that may be controlled include both monocotyledonous and dicotyledonous weeds, such as, for example, Alisma spp, Leptochloa, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus and especially Cyperus iria, Abutilon, Sida, Xanthium, Amaranthus, Chenopodium, Ipomoea, Bidens, Euphorbia, Chrysanthemum, Galium, Viola, Veronica, Ischaemumm Sida, Polygonum, Helianthus, Panicum, Eriochloa, Brachiaria, Cenchrus, Commelina, Spermacoce, Senna, Tridax, Richardia, Chamaesyce, and Conyza spp.

The rates of application of compounds of formula (I) may vary within wide limits and depend on the nature of the soil, the method of application (pre- or post-emergence; seed dressing; application to the seed furrow; no tillage application etc.), the crop plant, or weed to be controlled, the prevailing climatic conditions, and other factors governed by the method of application, the time of application and the target crop. The compounds of formula I according to the invention are generally applied at a rate of from 10 to 2000 g/ha, especially from 50 to 1000 g/ha, however, they may be also be used at even lower rates, e.g. from 5 to 2000 g/ha, and more preferably from 25 to 1000 g/ha.

Any method of application to weeds/crop of useful plant, or locus thereof, which is routinely used in agriculture may be used, for example application by spray or broadcast method typically after suitable dilution of a compound of formula (I) (whether said compound is formulated and/or in combination with one or more further active ingredients and/or safeners, as described herein).

The compounds of formula (I) according to the invention can also be used in combination with other active ingredients, e.g. other herbicides, and/or insecticides, and/or acaricides, and/or nematocides, and/or molluscicides, and/or fungicides, and/or plant growth regulators. Such mixtures, and the use of such mixtures to control weeds and/or undesired plant growth form yet further aspects of the invention. For the avoidance of doubt, mixtures of invention also include mixtures of two or more different compounds of formula (I).

Where a compound of formula (I) is combined with at least one additional herbicide, the following mixtures of the compound of formula (I) are particularly preferred. Compound of formula (I)+acetochlor, compound of formula (I)+acifluorfen, compound of formula (I)+acifluorfen-sodium, compound of formula (I)+aclonifen, compound of formula (I)+acrolein, compound of formula (I)+alachlor, compound of formula (I)+alloxydim, compound of formula (I)+allyl alcohol, compound of formula (I)+ametryn, compound of formula (I)+amicarbazone, compound of formula (I)+amidosulfuron, compound of formula (I)+aminopyralid, compound of formula (I)+aminocyclopyrachlor, compound of formula (I)+amitrole, compound of formula (I)+ammonium sulfamate, compound of formula (I)+anilofos, compound of formula (I)+asulam, compound of formula (I)+atrazine, formula (I)+aviglycine, formula (I)+azafenidin, compound of formula (I)+azimsulfuron, compound of formula (I)+BCPC, compound of formula (I)+beflubutamid, compound of formula (I)+benazolin, formula (I)+bencarbazone, compound of formula (I)+benfluralin, compound of formula (I)+benfuresate, compound of formula (I)+bensulfuron, compound of formula (I)+bensulfuron-methyl, compound of formula (I)+bensulide, compound of formula (I)+bentazone, compound of formula (I)+benzfendizone, compound of formula (I)+benzobicyclon, compound of formula (I)+benzofenap, compound of formula (I)+bifenox, compound of formula (I)+bilanafos, compound of formula (I)+bispyribac, compound of formula (I)+bispyribac-sodium, compound of formula (I)+borax, compound of formula (I)+bromacil, compound of formula (I)+bromobutide, formula (I)+bromophenoxim, compound of formula (I)+bromoxynil, compound of formula (I)+butachlor, compound of formula (I)+butafenacil, compound of formula (I)+butamifos, compound of formula (I)+butralin, compound of formula (I)+butroxydim, compound of formula (I)+butylate, compound of formula (I)+cacodylic acid, compound of formula (I)+calcium chlorate, compound of formula (I)+cafenstrole, compound of formula (I)+carbetamide, compound of formula (I)+carfentrazone, compound of formula (I)+carfentrazone-ethyl, compound of formula (I)+CDEA, compound of formula (I)+CEPC, compound of formula (I)+chlorflurenol, compound of formula (I)+chlorflurenol-methyl, compound of formula (I)+chloridazon, compound of formula (I)+chlorimuron, compound of formula (I)+chlorimuron-ethyl, compound of formula (I)+chloroacetic acid, compound of formula (I)+chlorotoluron, compound of formula (I)+chlorpropham, compound of formula (I)+chlorsulfuron, compound of formula (I)+chlorthal, compound of formula (I)+chlorthal-dimethyl, compound of formula (I)+cinidon-ethyl, compound of formula (I)+cinmethylin, compound of formula (I)+cinosulfuron, compound of formula (I)+cisanilide, compound of formula (I)+clethodim, compound of formula (I)+clodinafop, compound of formula (I)+clodinafop-propargyl, compound of formula (I)+clomazone, compound of formula (I)+clomeprop, compound of formula (I)+clopyralid, compound of formula (I)+cloransulam, compound of formula (I)+cloransulam-methyl, compound of formula (I)+CMA, compound of formula (I)+4-CPB, compound of formula (I)+CPMF, compound of formula (I)+4-CPP, compound of formula (I)+CPPC, compound of formula (I)+cresol, compound of formula (I)+cumyluron, compound of formula (I)+cyanamide, compound of formula (I)+cyanazine, compound of formula (I)+cycloate, compound of formula (I)+cyclosulfamuron, compound of formula (I)+cycloxydim, compound of formula (I)+cyhalofop, compound of formula (I)+cyhalofop-butyl, compound of formula (I)+2,4-D, compound of formula (I)+3,4-DA, compound of formula (I)+daimuron, compound of formula (I)+dalapon, compound of formula (I)+dazomet, compound of formula (I)+2,4-DB, compound of formula (I)+3,4-DB, compound of formula (I)+2,4-DEB, compound of formula (I)+desmedipham, formula (I)+desmetryn, compound of formula (I)+dicamba, compound of formula (I)+dichlobenil, compound of formula (I)+orthodichlorobenzene, compound of formula (I)+para-dichlorobenzene, compound of formula (I)+dichlorprop, compound of formula (I)+dichlorprop-P, compound of formula (I)+diclofop, compound of formula (I)+diclofop-methyl, compound of formula (I)+diclosulam, compound of formula (I)+difenzoquat, compound of formula (I)+difenzoquat metilsulfate, compound of formula (I)+diflufenican, compound of formula (I)+diflufenzopyr, compound of formula (I)+dimefuron, compound of formula (I)+dimepiperate, compound of formula (I)+dimethachlor, compound of formula (I)+dimethametryn, compound of formula (I)+dimethenamid, compound of formula (I)+dimethenamid-P, compound of formula (I)+dimethipin, compound of formula (I)+dimethylarsinic acid, compound of formula (I)+dinitramine, compound of formula (I)+dinoterb, compound of formula (I)+diphenamid, formula (I)+dipropetryn, compound of formula (I)+diquat, compound of formula (I)+diquat dibromide, compound of formula (I)+dithiopyr, compound of formula (I)+diuron, compound of formula (I)+DNOC, compound of formula (I)+3,4-DP, compound of formula (I)+DSMA, compound of formula (I)+EBEP, compound of formula (I)+endothal, compound of formula (I)+EPTC, compound of formula (I)+esprocarb, compound of formula (I)+ethalfluralin, compound of formula (I)+ethametsulfuron, compound of formula (I)+ethametsulfuron-methyl, formula (I)+ethephon, compound of formula (I)+ethofumesate, compound of formula (I)+ethoxyfen, compound of formula (I)+ethoxysulfuron, compound of formula (I)+etobenzanid, compound of formula (I)+fenoxaprop, compound of formula (I)+fenoxaprop-P, compound of formula (I)+fenoxaprop-ethyl, compound of formula (I)+fenoxaprop-P-ethyl, compound of formula (I)+fentrazamide, compound of formula (I)+ferrous sulfate, compound of formula (I)+flamprop-M, compound of formula (I)+flazasulfuron, compound of formula (I)+florasulam, compound of formula (I)+fluazifop, compound of formula (I)+fluazifop-butyl, compound of formula (I)+fluazifop-P, compound of formula (I)+fluazifop-P-butyl, formula (I)+fluazolate, compound of formula (I)+flucarbazone, compound of formula (I)+flucarbazone-sodium, compound of formula (I)+flucetosulfuron, compound of formula (I)+fluchloralin, compound of formula (I)+flufenacet, compound of formula (I)+flufenpyr, compound of formula (I)+flufenpyr-ethyl, formula (I)+flumetralin, compound of formula (I)+flumetsulam, compound of formula (I)+flumiclorac, compound of formula (I)+flumiclorac-pentyl, compound of formula (I)+flumioxazin, formula (I)+flumipropin, compound of formula (I)+fluometuron, compound of formula (I)+fluoroglycofen, compound of formula (I)+fluoroglycofen-ethyl, formula (I)+fluoxaprop, formula (I)+flupoxam, formula (I)+flupropacil, compound of formula (I)+flupropanate, compound of formula (I)+flupyrsulfuron, compound of formula (I)+flupyrsulfuron-methyl-sodium, compound of formula (I)+flurenol, compound of formula (I)+fluridone, compound of formula (I)+fluorochloridone, compound of formula (I)+fluoroxypyr, compound of formula (I)+flurtamone, compound of formula (I)+fluthiacet, compound of formula (I)+fluthiacet-methyl, compound of formula (I)+fomesafen, compound of formula (I)+foramsulfuron, compound of formula (I)+fosamine, compound of formula (I)+glufosinate, compound of formula (I)+glufosinate-ammonium, compound of formula (I)+glyphosate, compound of formula (I)+halosulfuron, compound of formula (I)+halosulfuron-methyl, compound of formula (I)+haloxyfop, compound of formula (I)+haloxyfop-P, compound of formula (I)+HC-252, compound of formula (I)+hexazinone, compound of formula (I)+imazamethabenz, compound of formula (I)+imazamethabenz-methyl, compound of formula (I)+imazamox, compound of formula (I)+imazapic, compound of formula (I)+imazapyr, compound of formula (I)+imazaquin, compound of formula (I)+imazethapyr, compound of formula (I)+imazosulfuron, compound of formula (I)+indanofan, compound of formula (I)+iodomethane, compound of formula (I)+iodosulfuron, compound of formula (I)+iodosulfuron-methyl-sodium, compound of formula (I)+ioxynil, compound of formula (I)+isoproturon, compound of formula (I)+isouron, compound of formula (I)+isoxaben, compound of formula (I)+isoxachlortole, compound of formula (I)+isoxaflutole, formula (I)+isoxapyrifop, compound of formula (I)+karbutilate, compound of formula (I)+lactofen, compound of formula (I)+lenacil, compound of formula (I)+linuron, compound of formula (I)+MAA, compound of formula (I)+MAMA, compound of formula (I)+MCPA, compound of formula (I)+MCPA-thioethyl, compound of formula (I)+MCPB, compound of formula (I)+mecoprop, compound of formula (I)+mecoprop-P, compound of formula (I)+mefenacet, compound of formula (I)+mefluidide, compound of formula (I)+mesosulfuron, compound of formula (I)+mesosulfuron-methyl, compound of formula (I)+mesotrione, compound of formula (I)+metam, compound of formula (I)+metamifop, compound of formula (I)+metamitron, compound of formula (I)+metazachlor, compound of formula (I)+methabenzthiazuron, formula (I)+methazole, compound of formula (I)+methylarsonic acid, compound of formula (I)+methyldymron, compound of formula (I)+methyl isothiocyanate, compound of formula (I)+metobenzuron, formula (I)+metobromuron, compound of formula (I)+metolachlor, compound of formula (I)+S-metolachlor, compound of formula (I)+metosulam, compound of formula (I)+metoxuron, compound of formula (I)+metribuzin, compound of formula (I)+metsulfuron, compound of formula (I)+metsulfuron-methyl, compound of formula (I)+MK-616, compound of formula (I)+molinate, compound of formula (I)+monolinuron, compound of formula (I)+MSMA, compound of formula (I)+naproanilide, compound of formula (I)+napropamide, compound of formula (I)+naptalam, formula (I)+NDA-402989, compound of formula (I)+neburon, compound of formula (I)+nicosulfuron, formula (I)+nipyraclofen, formula (I)+n-methyl glyphosate, compound of formula (I)+nonanoic acid, compound of formula (I)+norflurazon, compound of formula (I)+oleic acid (fatty acids), compound of formula (I)+orbencarb, compound of formula (I)+orthosulfamuron, compound of formula (I)+oryzalin, compound of formula (I)+oxadiargyl, compound of formula (I)+oxadiazon, compound of formula (I)+oxasulfuron, compound of formula (I)+oxaziclomefone, compound of formula (I)+oxyfluorfen, compound of formula (I)+paraquat, compound of formula (I)+paraquat dichloride, compound of formula (I)+pebulate, compound of formula (I)+pendimethalin, compound of formula (I)+penoxsulam, compound of formula (I)+pentachlorophenol, compound of formula (I)+pentanochlor, compound of formula (I)+pentoxazone, compound of formula (I)+pethoxamid, compound of formula (I)+petrolium oils, compound of formula (I)+phenmedipham, compound of formula (I)+phenmedipham-ethyl, compound of formula (I)+picloram, compound of formula (I)+picolinafen, compound of formula (I)+pinoxaden, compound of formula (I)+piperophos, compound of formula (I)+potassium arsenite, compound of formula (I)+potassium azide, compound of formula (I)+pretilachlor, compound of formula (I)+primisulfuron, compound of formula (I)+primisulfuron-methyl, compound of formula (I)+prodiamine, compound of formula (I)+profluazol, compound of formula (I)+profoxydim, formula (I)+prohexadione-calcium, compound of formula (I)+prometon, compound of formula (I)+prometryn, compound of formula (I)+propachlor, compound of formula (I)+propanil, compound of formula (I)+propaquizafop, compound of formula (I)+propazine, compound of formula (I)+propham, compound of formula (I)+propisochlor, compound of formula (I)+propoxycarbazone, compound of formula (I)+propoxycarbazone-sodium, compound of formula (I)+propyzamide, compound of formula (I)+prosulfocarb, compound of formula (I)+prosulfuron, compound of formula (I)+pyraclonil, compound of formula (I)+pyraflufen, compound of formula (I)+pyraflufen-ethyl, formula (I)+pyrasulfotole, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyributicarb, compound of formula (I)+pyridafol, compound of formula (I)+pyridate, compound of formula (I)+pyriftalid, compound of formula (I)+pyriminobac, compound of formula (I)+pyriminobac-methyl, compound of formula (I)+pyrimisulfan, compound of formula (I)+pyrithiobac, compound of formula (I)+pyrithiobac-sodium, formula (I)+pyroxasulfone, formula (I)+pyroxulam, compound of formula (I)+quinclorac, compound of formula (I)+quinmerac, compound of formula (I)+quinoclamine, compound of formula (I)+quizalofop, compound of formula (I)+quizalofop-P, compound of formula (I)+quizalofop-ethyl, compound of formula (I)+quizalofop-P-ethyl, compound of formula (I)+rimsulfuron, compound of formula (I)+sethoxydim, compound of formula (I)+siduron, compound of formula (I)+simazine, compound of formula (I)+simetryn, compound of formula (I)+SMA, compound of formula (I)+sodium arsenite, compound of formula (I)+sodium azide, compound of formula (I)+sodium chlorate, compound of formula (I)+sulcotrione, compound of formula (I)+sulfentrazone, compound of formula (I)+sulfometuron, compound of formula (I)+sulfometuron-methyl, compound of formula (I)+sulfosate, compound of formula (I)+sulfosulfuron, compound of formula (I)+sulfuric acid, compound of formula (I)+tar oils, compound of formula (I)+2,3,6-TBA, compound of formula (I)+TCA, compound of formula (I)+TCA-sodium, formula (I)+tebutam, compound of formula (I)+tebuthiuron, formula (I)+tefuryltrione, compound of formula 1+tembotrione, compound of formula (I)+tepraloxydim, compound of formula (I)+terbacil, compound of formula (I)+terbumeton, compound of formula (I)+terbuthylazine, compound of formula (I)+terbutryn, compound of formula (I)+thenylchlor, compound of formula (I)+thiazafluoron, compound of formula (I)+thiazopyr, compound of formula (I)+thifensulfuron, compound of formula (I)+thiencarbazone, compound of formula (I)+thifensulfuron-methyl, compound of formula (I)+thiobencarb, compound of formula (I)+tiocarbazil, compound of formula (I)+topramezone, compound of formula (I)+tralkoxydim, compound of formula (I)+tri-allate, compound of formula (I)+triasulfuron, compound of formula (I)+triaziflam, compound of formula (I)+tribenuron, compound of formula (I)+tribenuron-methyl, compound of formula (I)+tricamba, compound of formula (I)+triclopyr, compound of formula (I)+trietazine, compound of formula (I)+trifloxysulfuron, compound of formula (I)+trifloxysulfuron-sodium, compound of formula (I)+trifluralin, compound of formula (I)+triflusulfuron, compound of formula (I)+triflusulfuron-methyl, compound of formula (I)+trifop, compound of formula (I)+trifop-methyl, compound of formula (I)+trihydroxytriazine, compound of formula (I)+trinexapac-ethyl, compound of formula (I)+tritosulfuron, compound of formula (I)+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6), compound of formula (I)+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), and compound of formula (I)+4-hydroxy-3-[[2-(3-methoxypropyl)-6-(difluoromethyl)-3-pyridinyl]carbonyl]-bicyclo[3.2.1]oct-3-en-2-one.

Whilst two-way mixtures of a compound of formula (I) and another herbicide are explicitly disclosed above, the skilled man will appreciate that the invention extends to three-way, and further multiple combinations comprising the above two-way mixtures.

In preferred embodiments a compound of formula (I) is combined with an acetolactate synthase inhibitor, (e.g. one or more of florasulam, metsulfuron, thifensulfuron, tribenuron, triasulfuron, flucarbazone, flupyrsulfuron, iodosulfuron, mesosulfuron, propoxicarbazone, sulfosulfuron, pyroxsulam and tritosulfuron, as well as salts or esters thereof), a synthetic auxin herbicide (e.g. one or more of aminocyclopyrachlor, aminopyralid, clopyralid, 2,4-D, 2,4-DB, dicamba, dichlorprop, fluoroxypyr, MCPA, MCPB, mecopropand mecoprop-P), an ACCase-inhibiting herbicide (e.g. one or more of phenylpyrazolin; pinoxaden; an aryloxyphenoxypropionic herbicide such as clodinafop, cyhalofop, diclofop, fenoxaprop, fluazifop, haloxyfop, quizalofop, trifop and mixtures thereof, as well as the isomers thereof, for example, fenoxaprop-P, fluazifop-P, haloxyfop-P, quizalofop-P; and a cyclohexanedione herbicide such as alloxydim; butroxydim, clethodim, cycloxydim, profoxydim, sethoxydim, tepraloxydim and tralkoxydim, as well as salts or esters thereof), and/or an auxin transport inhibitor such as semicarbazone (e.g. diflufenzopyr, in particular the sodium salt) or phthalamate compound (e.g. naptalam).

Particularly preferred mixture partners for compounds of formula (I) are: florasulam, iodosulfuron-methyl-sodium, mesosulfuron-methyl, metsulfuron-methyl, thifensulfuron, triasulfuron, tribenuron-methyl or pyroxsulam; dicamba, fluoroxypyr, MCPA, mecoprop or mecoprop-P; clodinafop-propargyl, cyhalofop-butyl, diclofop-methyl, fenoxaprop-ethyl, fenoxaprop-P-ethyl, fluazifop-butyl, fluazifop-P-butyl, haloxyfop-methyl, haloxyfop-P-methyl, pinoxaden, propaquizafop, quizalofop-ethyl, quizalofop-P-ethyl, tralkoxydim, trifop-methyl, diflufenzopyr-Na, and naptalam.

For the avoidance of doubt, even if not explicitly stated above, the mixing partners of the compound of formula (I) may also be in the form of any suitable agrochemically acceptable ester or salt, as mentioned e.g. in The Pesticide Manual, Thirteenth Edition, British Crop Protection Council, 2003.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

The compounds of formula (I) according to the invention can also be used in combination with one or more safeners. Likewise, mixtures of a compound of formula (I) according to the invention with one or more further active ingredients, in particular with one or more further herbicides, can also be used in combination with one or more safeners. Suitable safeners for use in combination with compounds of formula (I) include AD 67 (MON 4660), benoxacor, cloquintocet-mexyl, cyometrinil and the corresponding (Z) isomer, cyprosulfamide (CAS RN 221667-31-8), dichlormid, fenchlorazole-ethyl, fenclorim, flurazole, fluxofenim, furilazole and the corresponding R isomer, isoxadifen-ethyl, mefenpyr-diethyl, oxabetrinil, naphthalic anhydride (CAS RN 81-84-5) and N-isopropyl-4-(2-methoxy-benzoylsulfamoyl)-benzamide (CAS RN 221668-34-4). Particularly preferred safeners for use in the invention are cloquintocet-mexyl, cyprosulfamide, fenchlorazole-ethyl and mefenpyr-diethyl. The safeners of the compound of formula (I) may also be in the form of esters or salts, as mentioned e.g. in The Pesticide Manual, 13^{th} Edition supra. The reference to cloquintocet-mexyl also applies to a lithium, sodium, potassium, calcium, magnesium, aluminium, iron, ammonium, quaternary ammonium, sulfonium or phosphonium salt thereof as disclosed in WO02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

Preferred mixtures of a compound of formula (I) with further herbicides and safeners include: a compound of formula (I)+pinoxaden+cloquinctocet-mexyl, a compound of formula (I)+clodinafop+cloquintocet-mexyl, and a compound of formula (I)+clodinafop-propargyl+cloquintocet-mexyl.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

### Example 1

### Synthesis of 4-amino-2,5-dichloro-6-methoxycarbonylpyrimidine

### 1.1 Preparation of 2,4-dihydroxy-6-methoxycarbonylpyrimidine (methyl orotate)

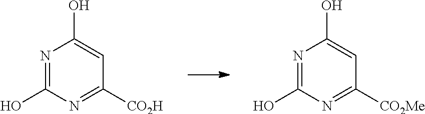

Thionyl chloride (500 ml), pyridine (2.5 ml) and a few drops of dimethylformamide were added to orotic acid monohydrate (78 g, 0.44 mol). The reaction mixture was stirred at ambient temperature for 5 days and then heated under reflux for an additional 14 hours. After cooling the solid material was allowed to settle and the supernatant decanted. The solid residue was washed with hexane and dried. Methanol (700 ml) was added dropwise with agitation to the solid. Once the rate of the gas formation slowed, the mixture was heated at reflux overnight and then cooled to 4-5° C. The solid was removed by filtration and washed with methyl alcohol and ether to provide methyl orotate (73 g, 97%). Characterising data for the compound are as follows: ¹H nmr (400 MHz, d₆-DMSO) δ_{H} 11.41 (1H, s), 11.26 (1H, s), 6.04 (1H, s), 3.84 (3H, s) ppm.

### 1.2 Preparation of 5-chloro-2,4-dihydroxy-6-methoxycarbonylpyrimidine

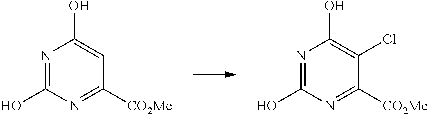

A catalytic quantity of ferric chloride was added to a solution of methyl orotate (34 g, 0.2 mol) in acetic anhydride (5% solution in glacial acetic acid, 500 ml). The mixture was heated to 90-95° C. and sulphuryl chloride (54 g, 0.40 mol) was added dropwise. After the addition was complete, the solution was slowly brought to reflux with agitation and heating was continued overnight. The solution was cooled to 18° C. and the solid was removed by filtration. The solid was washed with acetic acid and then with water, and dried to give 5-chloro-2,4-dihydroxy-6-methoxycarbonylpyrimidine (36.0 g, 89%). Characterising data for this compound are as follows: ¹H nmr (400 MHz, d₆-DMSO) δ_{H} 11.86 (1H, s), 11.62 (1H, s), 3.88 (3H, s) ppm.

### 1.3 Preparation of 6-methoxycarbonyl-2,4,5-trichloropyrimidine

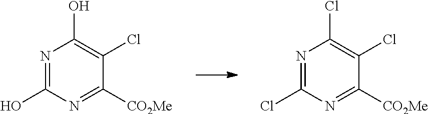

Phosphorus oxychloride (993 ml) was added to 5-chloro-2,4-dihydroxy-6-methoxycarbonylpyrimidine (30.0 g, 0.146 mol) at 10° C. and the resulting solution cooled to 0° C. N,N-Diethyl aniline (30.9 ml, 0.193 mol) was added dropwise to the stirred solution. After the addition was complete, the reaction mixture was allowed to warm slowly to ambient temperature and was then heated at reflux overnight. The resulting solution was cooled and concentrated under reduced pressure. The residue was poured onto crushed ice (600 g) and extracted with cold ether. The ether extracts were washed with brine, dried over sodium sulphate, filtered and evaporated under reduced pressure to give a light brown solid. This was triturated with warm hexane to yield 6-methoxycarbonyl-2,4,5-trichloropyrimidine (28 g, 82%). Characterising data for this compound are as follows: ¹H nmr (400 MHz, CDCl₃) ≡_{H} 4.02 (3H, s) ppm.

### 1.4 Preparation of 4-amino-2,5-dichloro-6-methoxycarbonylpyrimidine

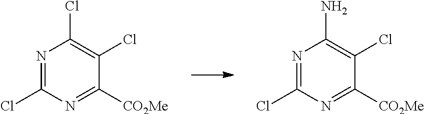

Aqueous ammonia (30% solution; 8.0 ml, 0.42 mol) was added dropwise to a stirred solution of 6-methoxycarbonyl-2,4,5-trichloropyrimidine (20.0 g, 0.083 mol) in THF (1000 ml) at 0° C. The reaction mixture was stirred at 0° C. for 1 hour and then filtered. The filtrate was evaporated under reduced pressure to give a white solid that was washed with twice with hexane and dried under vacuum to provide 4-amino-2,5-dichloro-6-methoxycarbonylpyrimidine (15.0 g, 82%). Characterising data are as follows: ¹H nmr (400 MHz, d₆-DMSO) δ_{H} 8.57 (1H, br s), 7.94 (1H, br s), 3.88 (3H, s) ppm.

### Example 2

### GENERAL Methodology for the Reaction of 4-amino-2,5-dichloro-6-methoxycarbonylpyrimidine with a Heteroaromatic Boronic Acid

Two different general methods are described below:
2.1 Palladium tetrakis(triphenylphosphine) (3 mg) was added to a suspension of 4-amino-2,5-dichloro-6-methoxycarbonylpyrimidine (prepared as described in example 1) (444 mg, 2.0 mmol), potassium carbonate (300 mg, 2.2 mmol) and a heteroaromatic boronic acid (2.2 mmol) in acetonitrile (4 ml) and dimethylacetamide (2 ml). The resulting mixture was heated in a microwave reactor at 170° C. for 15 minutes, then allowed to cool and poured into water. The resulting mixture was extracted with ethyl acetate and the organic extracts washed with water, dried over magnesium sulphate, filtered and evaporated under reduced pressure. The residue was purified by chromatography on silica, with ethyl acetate/hexane mixtures as eluent to provide the desired product.
2.2 Palladium tetrakis(triphenylphosphine) (3 mg) was added to a suspension of 4-amino-2,5-dichloro-6-methoxycarbonylpyrimidine (prepared as described in example 1) (444 mg, 2.0 mmol), triethylamine (1 ml) and a heteroaromatic boronic acid (2.2 mmol) in acetonitrile (4 ml). The resulting mixture was heated in a microwave reactor at 170° C. for 15 minutes, then allowed to cool and the solvent evaporated under reduced pressure. The residue was purified by chromatography on silica, with ethyl acetate/hexane mixtures as eluent to provide the desired product.

### Example 3

### General Methodology for the Reaction of 4-amino-2,5-dichloro-6-methoxycarbonylpyrimidine with a Heteroaromatic Boronate Ester

The boronate ester (0.5 mmol), 4-amino-2,5-dichloro-6-methoxycarbonylpyrimidine (prepared as described in example 1) (110 mg, 0.5 mmol), caesium fluoride (151 mg, 1.0 mmol) and [1,1′-bis(diphenylphosphino)-ferrocene]dichloropalladium (II) complex with dichloromethane (1:1) (41 mg, 0.05 mmol) were placed in a vial. The vial was evacuated and backfilled with nitrogen before adding dimethoxyethane (1 ml) and water (1 ml). The reaction mixture was heated in a microwave reactor at 140° C. for 10 minutes, then allowed to cool, filtered and extracted with ethyl acetate (10 ml). The organic extract was washed with water (10 ml) and brine (10 ml), dried over magnesium sulphate, filtered and evaporated under reduced pressure. The crude product was purified using an SCX-2 column (catch and release method) washing with methanol, then 2N ammonia in methanol. The product was further purified by chromatography on silica with hexane/ethyl acetate/triethylamine mixtures as eluent.

### Example 4

### General Methodology for the Preparation of a Heteroaromatic Boronate Ester from a Heteroaromatic Boronic Acid

A suspension of a heteroaromatic boronic acid (1.8 mmol), 1,3-propanediol (0.26 ml, 3.6 mmol) and 4 Å molecular sieves in toluene (10 ml) was heated at reflux for 4 hours, then cooled to room temperature. The mixture was filtered and the filtrate concentrated under reduced pressure. The residue was slurried in dichloromethane (10 ml) and washed with water (2×10 ml). The organic phase was dried over magnesium sulphate, filtered and evaporated under reduced pressure to yield the desired boronate ester.

### Example 5

### Synthesis of 4-amino-5-chloro-6-methoxycarbonyl-2-(5-methylbenz-[b]thiophen-3-yl)-pyrimidine (compound 2-57)

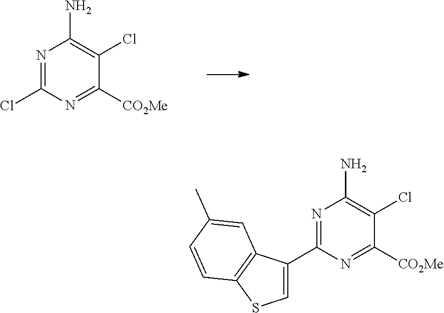

5-Methyl-benzo[b]thiophen-3-ylboronic acid (96 mg, 0.5 mmol), 4-amino-2,5-dichloro-6-methoxycarbonylpyrimidine (prepared as described in example 1) (110 mg, 0.5 mmol) and palladium tetrakis(triphenylphosphine) (58 mg, 0.05 mmol) were placed in a vial. The vial was evacuated and backfilled with nitrogen before adding triethylamine (77 μl, 0.55 mmol) and acetonitrile (4 ml). The reaction mixture was heated in a microwave reactor at 140° C. for 10 minutes, then allowed to cool. The reaction mixture was filtered and concentrated under reduced pressure to give a brown oil, which was purified by chromatography on silica with a hexane/ethyl acetate gradient as eluent to yield 4-amino-5-chloro-6-methoxycarbonyl-2-(5-methyl benzothiophen-3-yl)-pyrimidine as an orange solid (33 mg, 20%). Characterising data for the compound are as follows: M.p. 164-165° C.; ¹H nmr (400 MHz, CDCl₃) δ_{H} 8.13 (1H, s), 7.73 (1H, d), 7.62 (1H, s), 7.22 (1H, d), 5.61 (2H, br s), 4.03 (3H, s), 2.47 (3H, s) ppm; R_{f} (7:3 hexane:ethyl acetate) 0.25.

### Example 6

### Synthesis of 4-amino-5-chloro-6-methoxycarbonyl-2-(1-methyl-1H-pyrazol-4-yl)-pyrimidine (compound 2-71)

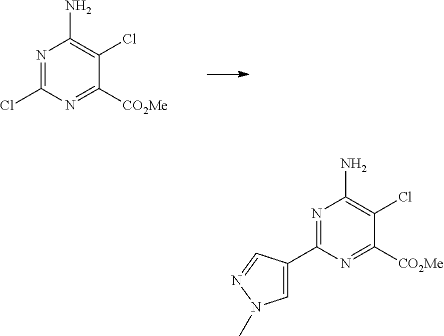

1-Methyl-4-(4,4,5,5-tetramethyl-1,3,2-dioxaborolan-2-yl)-1H-pyrazole (104 mg, 0.5 mmol), 4-amino-2,5-dichloro-6-methoxycarbonylpyrimidine (prepared as described in example 1) (110 mg, 0.5 mmol), caesium fluoride (151 mg, 1.0 mmol) and bis(triphenylphosphine) palladium (II) chloride (35 mg, 0.05 mmol) were placed in a vial. The vial was evacuated and backfilled with nitrogen before adding dimethoxyethane (1 ml) and water (1 ml). The reaction mixture was then heated in a microwave reactor at 140° C. for 10 minutes and allowed to cool. The reaction mixture was filtered and extracted with ethyl acetate (10 ml). The organic extract was washed with water (10 ml) and brine (10 ml), dried over magnesium sulphate, filtered and evaporated under reduced pressure to provide a pale yellow solid, which was purified by chromatography on silica, using a hexane/ethyl acetate gradient as eluent. Further purification was carried out by chromatography on silica with a dichloromethane/ethyl acetate gradient as eluent to yield 4-amino-5-chloro-6-methoxycarbonyl-2-(1-methyl-1H-pyrazol-4-yl)-pyrimidine as an off white solid (60 mg, 45%). Characterising data are as follows: M.p. 185-186° C.; ¹H nmr (400 MHz, CDCl₃) δ_{H} 8.07 (1H, s), 8.02 (1H, s), 5.47 (2H, br s), 4.00 (3H, s), 3.93 (3H, s) ppm; R_{f} (4:6 dichloromethane:ethyl acetate) 0.2.

### Example 7

### Synthesis of 4-amino-5-chloro-2-(2-cyanopyridin-5-yl)-6-methoxycarbonylpyrimidine (compound 2-158)

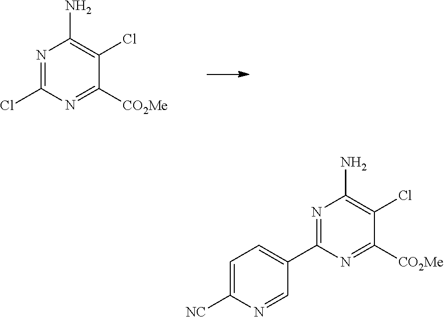

2-Cyanopyridine-5-boronic acid pinacol ester (101 mg, 0.5 mmol), 4-amino-2,5-dichloro-6-methoxycarbonylpyrimidine (prepared as described in example 1) (110 mg, 0.5 mmol), caesium fluoride (151 mg, 1.0 mmol) and bis(triphenylphosphine) palladium (II) chloride (35 mg, 0.05 mmol) were placed in a vial. The vial was evacuated and backfilled with nitrogen before adding dimethoxyethane (1 ml) and water (1 ml). The reaction mixture was heated in a microwave reactor at 140° C. for 10 minutes, then allowed to cool. The reaction mixture was filtered and extracted with ethyl acetate (10 ml). The organic extract was washed with water (10 ml) and brine (10 ml), dried over magnesium sulphate, filtered and evaporated under reduced pressure to give a brown oil, which was purified by chromatography on silica with a hexane/ethyl acetate gradient as eluent. Further purification was carried out using an SCX-2 column (catch and release method). The column was washed with 1 CV (column volume) methanol before loading the crude product as a solution in methanol. Further washes were carried out with 2 CV methanol followed by 2 CV 2M ammonia in methanol to provide 4-amino-5-chloro-2-(2-cyanopyridin-5-yl)-6-methoxycarbonylpyrimidine as an off white solid (39 mg, 27%). Characterising data are as follows: M.p. 210-213° C.; ¹H nmr (400 MHz, CDCl₃) δ_{H} 9.62 (1H, d), 8.76 (1H, dd), 7.77 (1H, d), 5.71 (2H, br s) 4.04 (3H, s) ppm; R_{f} (1:1 hexane:ethyl acetate) 0.3.

Other compounds that were also prepared using the methods described in Examples 2, 3 and 4 are listed in Table 2

TABLE 2
Compounds made as described in Examples 2, 3, and 4 above.
Characterising data is either melting point (° C.), or
¹H nmr (400 MHz, CDCl₃) δ_{H} ppm, or m/z (LCMS)

| Compound | | | |
|---|---|---|---|
| Number | Name | Structure | Characterising data: |
| 2-154 | 4-Amino-5- chloro-6- methoxycarbonyl- 2-(2- methoxypyridin- 3-yl)-pyrimidine | 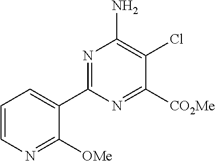 | 8.3 (1 H, dd), 8.0 (1 H, dd), 7.0 (1 H, dd), 5.65 (2 H, br s), 4.0 (6 H, s) |
| 2-155 | 4-Amino-5- chloro-6- methoxycarbonyl- 2-(2- methoxypyridin- 5-yl)-pyrimidine | 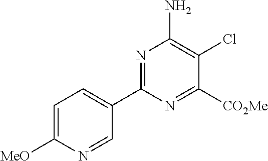 | 9.15 (1 H, d), 8.5 (1 H, dd), 6.8 (1 H, d), 5.6 (2 H, br s), 4.05 (3 H, s), 4.0 (3 H, s) |
| 2-159 | 4-Amino-5- chloro-2-(2,6- dimethoxypyridin- 3-yl)-6- methoxycarbonyl pyrimidine | 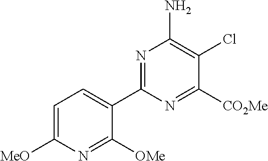 | 8.1 (1 H, d), 6.4 (1 H, d), 5.6 (2 H, br s), 4.05 (3 H, s), 4.00 (3 H, s), 3.95 (3 H, s) |
| 2-182 | 4-Amino-5- chloro-6- methoxycarbonyl- 2-(2- methoxypyrimidin- 5-yl)-pyrimidine | 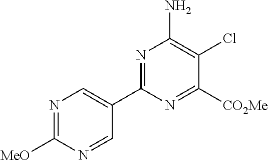 | 9.4 (2 H, s), 5.8 (2 H, br s), 4.1 (3 H, s), 4.0 (3 H, s) |
| 2-157 | 4-Amino-5- chloro-2-(2- fluoropyridin-5- yl)-6- methoxycarbonyl pyrimidine | 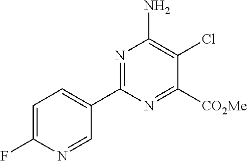 | 9.2 (1 H, d), 8.7 (1 H, m), 7.0 (1 H, dd), 5.7 (2 H, br s), 4.05 (3 H, s) |
| 2-156 | 4-Amino-5- chloro-2-(2- chloropyridin-5- yl)-6- methoxycarbonyl pyrimidine | 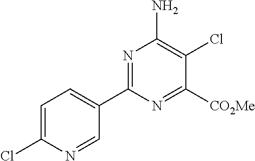 | 9.3 (1 H, d), 8.6 (1 H, dd), 7.4 (1 H, d), 5.65 (2 H, br s), 4.05 (3 H, s) |
| 2-153 | 4-Amino-5- chloro-6- methoxycarbonyl- 2-pyridin-3- ylpyrimidine | 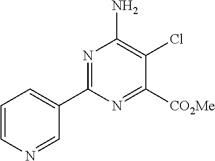 | 138-140 |
| 2-196 | 4-Amino-5- chloro-2- isoquinolin-4-yl-6- methoxycarbonyl pyrimidine | 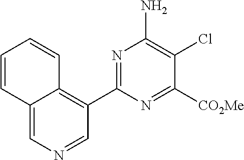 | 145-146 |
| 2-2 | 4-Amino-5- chloro-2-(5- chlorothiophen-2- yl)-6- methoxycarbonyl pyrimidine | 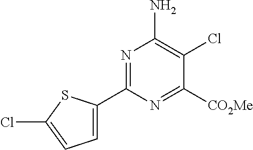 | M + H⁺ 304, 306, 308 |
| 2-1 | 4-Amino-5- chloro-6- methoxycarbonyl- 2-thiophen-2- ylpyrimidine | 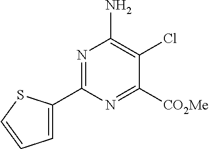 | M + H⁺ 270, 272 |
| 2-3 | 4-Amino-5- chloro-2-(5- cyanothiophen-2- yl)-6- methoxycarbonyl pyrimidine | 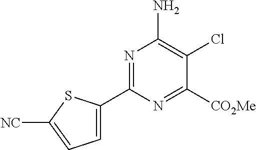 | M + H⁺ 295, 297 |
| 2-161 | 4-Amino-5- chloro-2-(2- fluoro-6- methylpyridin-3- yl)-6- methoxycarbonyl pyrimidine | 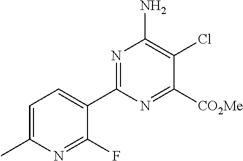 | 8.4 (1 H, dd), 7.1 (1 H, dd), 5.7 (2 H, br s), 4.0 (3 H, s), 2.5 (3 H, s) |
| 2-183 | 4-Amino-5- chloro-2-(2,4- dimethoxypyrimidin- 5-yl)-6- methoxycarbonyl pyrimidine | 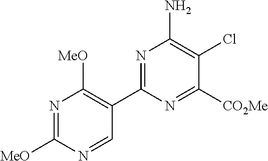 | 9.0 (1 H, s), 5.9 (2 H, br s), 4.16 (3 H, s), 4.18 (3 H, s), 4.05 (3 H, s) |
| 2-172 | 4-Amino-5- chloro-2-(2- fluoropyridin-3- yl)-6- methoxycarbonyl pyrimidine | 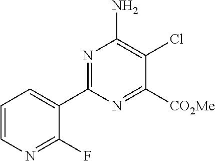 | 8.5 (1 H, m), 8.3 (1 H, m), 7.3 (1 H, m), 5.7 (2 H, br s), 4.0 (3 H, s) |
| 2-16 | 4-Amino-5- chloro-6- methoxycarbonyl- 2-thiophen-3- ylpyrimidine | 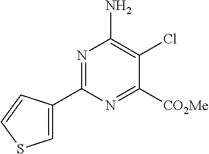 | 8.3 (1 H, d), 7.8 (1 H, dd), 7.3 (1 H, m), 6.1 (2 H, br s), 4.0 (3H, s) |
| 2-9 | 4-Amino-5- chloro-6- methoxycarbonyl- 2-(5- methylcarbonyl- thiophen-2-yl)- pyrimidine | 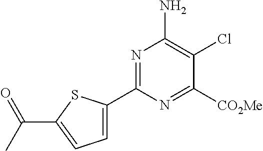 | 7.9 (1 H, d), 7.7 (1 H, d), 5.8 (2 H, br s), 4.0 (3 H, s), 2.6 (3 H, s) |
| 2-56 | 4-Amino-2- (benzothiophen- 3-yl)-5-chloro-6- methoxycarbonyl pyrimidine | 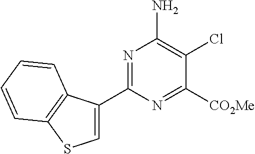 | 9.0 (1 H, dd), 8.5 (1 H, s), 7.9 (1 H, dd), 7.5 (1 H, m), 7.4 (1 H, m), 5.6 (2 H, br s), 4.1 (3 H, s) |
| 2-54 | 4-Amino-2- (benzothiophen- 2-yl)-5-chloro-6- methoxycarbonyl pyrimidine | 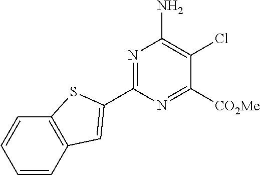 | 8.2 (1 H, s), 7.8 (2 H, m), 7.4 (2 H, m), 5.6 (2 H, br s), 4.0 (3 H, s) |
| 2-86 | 4-Amino-5- chloro-2-(3,5- dimethylisoxazol- 4-yl)-6- methoxycarbonyl pyrimidine | 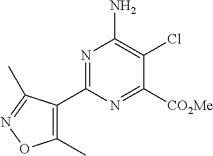 | 5.5 (2 H, br s), 4.0 (3 H, s), 2.7 (3 H, s), 2.5 (3 H, s) |
| 2-17 | 4-Amino-5- chloro-6- methoxycarbonyl- 2-(4- methylthiophen- 3-yl)-pyrimidine | 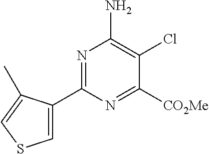 | 8.2 (1 H, s), 7.0 (1 H, s), 6.0 (2 H, br s), 4.0 (3 H, s), 2.6 (3 H, s) |
| 2-55 | 4-Amino-5- chloro-6- methoxycarbonyl- 2-(5- methylbenzothiophen- 2-yl)- pyrimidine | 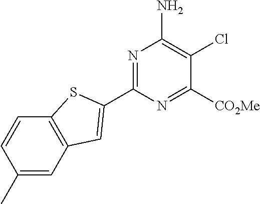 | 8.2 (1 H, s), 7.7 (1 H, d), 7.6 (1 H, s), 7.2 (1 H, d), 5.7 (2 H, br s), 4.0 (3 H, s), 2.5 (3 H, s) |
| 2-18 | 4-Amino-5- chloro-2-(2- chlorothiophen-3- yl)-6- methoxycarbonyl pyrimidine | 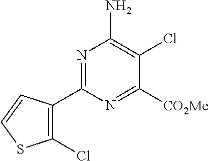 | 7.5 (1 H, d), 7.1 (1 H, d), 6.0 (2 H, br s), 4.0 (3 H, s) |
| 2-170 | 4-Amino-5- chloro-6- methoxycarbonyl- 2-(6-[morpholin- 1-yl]-pyridin-3-yl)- pyrimidine | 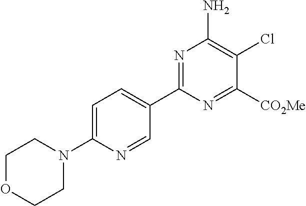 | 9.2 (1 H, d), 8.8 (1 H, dd), 6.9 (1 H, d), 5.9 (2 H, br s), 4.0 (3 H, s), 3.9 (4 H, m), 3.8 (4 H, m) |
| 2-166 | 4-Amino-5- chloro-6- methoxycarbonyl- 2-(2-[2,2,2- trifluoroethoxy]- pyridin-3-yl)- pyrimidine | 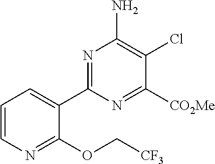 | 8.4 (1 H, m), 8.3 (1 H, m), 7.2 (1 H, m), 7.0 (2 H, br s), 4.8 (2 H, q), 4.0 (3 H, s) |
| 2-34 | 4-Amino-5- chloro-2-(furan-3- yl)-6- methoxycarbonyl pyrimidine | 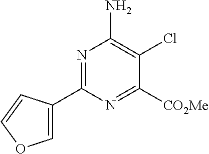 | 8.2 (1 H, s), 7.4 (1 H, d), 6.9 (1 H, d), 5.5 (2H, br s), 4.0 (3 H, s) |
| 2-70 | 4-Amino-5- chloro-6- methoxycarbonyl- 2-(pyrazol-4-yl)- pyrimidine | 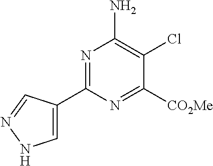 | 8.1 (1 H, s), 7.2 (1 H, s), 4.1 (3 H, s), 3.9 (1 H, br s), 3.2 (2 H, br s) |
| 2-72 | 4-Amino-5- chloro-2-(3,5- dimethylpyrazol- 4-yl)-6- methoxycarbonyl pyrimidine | 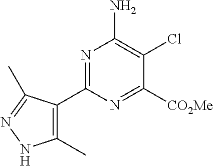 | 5.8 (2 H, br s), 5.5 (1 H, br s), 4.0 (3 H, s), 2.6 (3 H, s), 2.4 (3 H, s) |
| 2-4 | 4-Amino-5- chloro-6- methoxycarbonyl- 2-(5- methylthiophen- 2-yl)-pyrimidine | 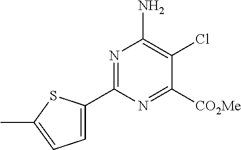 | 7.7 (1 H, d), 6.8 (1 H, m), 6.0 (2 H, br s), 4.0 (3 H, s), 2.5 (3 H, s) |
| 2-173 | 4-Amino-5- chloro-6- methoxycarbonyl- 2-(6- methylcarbonyl- aminopyridin-3-yl)- pyrimidine | 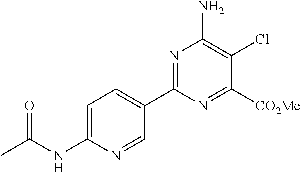 | 13.0 (1 H, s), 9.2 (1 H, d), 9.1 (1 H, dd), 8.7 (1 H, d), 5.7 (2 H, br s), 4.0 (3 H, s), 2.4 (3 H, s) |
| 2-168 | 4-Amino-5- chloro-6- methoxycarbonyl- 2-(6-nitropyridin- 3-yl)-pyrimidine | 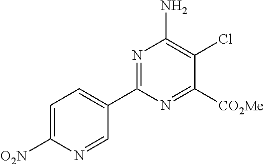 | 9.5 (1 H, d), 9.0 (1 H, dd), 8.3 (1 H, d), 5.7 (2 H, br s), 4.1 (3 H, s) |
| 2-7 | 4-Amino-5- chloro-6- methoxycarbonyl- 2-(5- methoxythiophen- 2-yl)-pyrimidine | 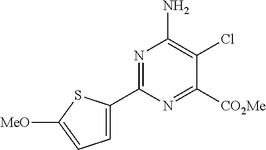 | 92-94 |
| 2-19 | 4-Amino-5- chloro-2-(2,5- dichlorothiophen- 3-yl)-6- methoxycarbonyl pyrimidine | 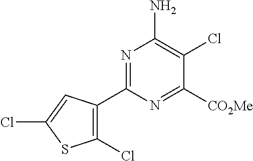 | 83-85 |
| 2-146 | 4-Amino-5- chloro-2- (imidazo[1,5- a]pyridin-3-yl)-6- methoxycarbonyl pyrimidine | 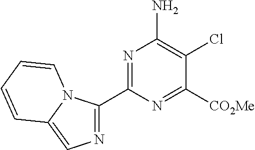 | 9.8 (1 H, d), 8.5 (1 H, s), 7.7 (1 H, d), 7.4 (1 H, t), 7.0 (1 H, t), 5.6 (2 H, br s), 4.1 (3 H, s) |
| 2-179 | 4-Amino-5- chloro-2-(2,6- dichloropyridin-4- yl)-6- methoxycarbonyl pyrimidine | 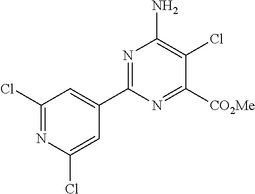 | 8.2 (2 H, s), 5.6 (2 H, br s), 4.0 (3 H, s) |

### Example 8

### Synthesis of 4-amino-2-(6-chloropyridin-3-yl)-5-ethenyl-6-methoxycarbonyl-pyrimidine (compound 68-156)

### 8.1 Preparation of 4-amino-5-chloro-6-methoxycarbonyl-2-methylthiopyrimidine

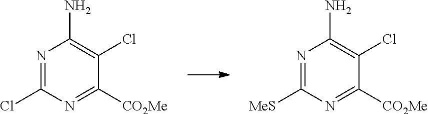

Sodium methanethiolate (3.0 g, 35 mmol) was added portionwise to a stirred solution of 4-amino-2,5-dichloro-6-methoxycarbonylpyrimidine (prepared as described in example 1) (4.4 g, 20 mmol) in methanol (100 ml) to give a pale yellow solution. The resulting mixture was stirred at reflux for 2 hours then allowed to cool for 2 hours, filtered and evaporated under reduced pressure. The residue was dissolved in water and ethyl acetate, the phases separated and the aqueous extracted with further ethyl acetate. The combined organic phases were washed with water and brine, dried over magnesium sulphate, filtered and evaporated under reduced pressure to provide 4-amino-5-chloro-6-methoxycarbonyl-2-methylthiopyrimidine as a yellow solid (2.2 g), which was used without further purification.

¹H nmr (400 MHz, CDCl₃) δ_{H} 5.55 (2H, br s), 3.95 (3H, s), 2.50 (3H, s) ppm.

### 8.2 Preparation of 4-amino-5-ethenyl-6-methoxycarbonyl-2-methylthiopyrimidine

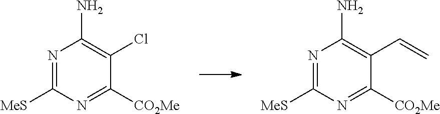

Water (2 ml) was added with stirring to a solution of 4-amino-5-chloro-6-methoxycarbonyl-2-methylthiopyrimidine (233 mg, 1.0 mmol), vinyl boronic acid pinacol ester (0.4 ml, 2.0 mmol), caesium fluoride (0.6 g, 1.0 mmol) and [1,1′-bis(diphenylphosphino)-ferrocene]dichloropalladium (II) complex with dichloromethane (1:1) (200 mg, 0.25 mmol) in dimethoxyethane (3 ml). The mixture was heated in a microwave reactor at 140° C. for 2 hours, then allowed to cool, diluted with ethyl acetate and washed with water and brine. The organic phase was dried over magnesium sulphate, filtered and evaporated under reduced pressure to provide a brown oil which was purified by chromatography on silica using hexane/ethyl acetate (4:1) as eluent to provide 4-amino-5-ethenyl-6-methoxycarbonyl-2-methylthiopyrimidine as a beige solid (120 mg, 50%).

¹H nmr (400 MHz, CDCl₃) δ_{H} 6.70 (1H, dd), 5.75 (2H, dd), 5.30 (2H, br s), 3.90 (3H, s), 2.50 (3H, s) ppm.

### 8.3 Preparation of 4-amino-2-(6-chloropyridin-3-yl)-5-ethenyl-6-methoxycarbonyl-pyrimidine (compound 68-156)

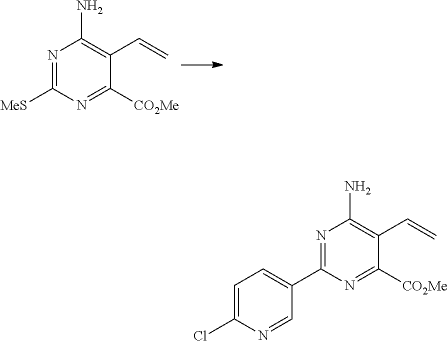

A solution of 4-amino-5-ethenyl-6-methoxycarbonyl-2-methylthiopyrimidine (113 mg, 0.50 mmol), 6-chloropyridine-3-boronic acid (85 mg, 0.55 mmol), copper thiophene-2-carboxylate (125 mg, 0.65 mmol), tri(2-furyl)phosphine (19 mg, 80 μmol) and tris(dibenzylideneacetone)dipalladium chloroform adduct (10 mg, 10 μmol) in tetrahydrofuran (3 ml) was heated in a microwave reactor at 100° C. for 30 minutes, then allowed to cool. Ether was added and the resulting solution washed with concentrated aqueous ammonia and brine, dried over magnesium sulphate, filtered and evaporated under reduced pressure to provide a yellow semi-solid (0.23 g). The crude product was purified by chromatography on silica using hexane/ethyl acetate (4:1) as eluent to provide 4-amino-2-(6-chloropyridin-3-yl)-5-ethenyl-6-methoxycarbonyl-pyrimidine as a white solid (40 mg, 27%). M.p. 194-195° C.; ¹H nmr (400 MHz, CDCl₃) δ_{H} 9.32 (1H, s), 8.60 (1H, d), 7.40 (1H, d), 6.80 (1H, dd), 5.65 (2H, m), 5.45 (2H, br s), 3.97 (3H, s) ppm.

### Example 9

### Synthesis of 4-amino-2-thiophen-2-ylpyrimidine-6-carboxylic acid

### 9.1 Preparation of 4-hydroxy-2-thiophen-2-ylpyrimidine-6-carboxylic acid

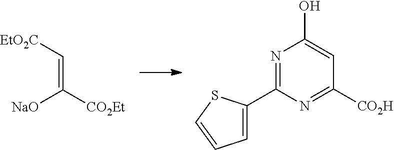

Sodium hydroxide (0.4 g, 10 mmol) was added to a stirred solution of diethyl oxaloacetate sodium salt (1.3 g, 6.2 mmol) in water (50 ml) and the mixture stirred for 30 minutes. 2-Thiopheneamidine (1.0 g, 7.9 mmol) was added to the solution and the mixture was heated at 70° C. overnight, then cooled to 0° C., acidified to pH1 and allowed to stand for an hour. The precipitate was isolated by filtration, washed with water and cold ethyl acetate and dried to yield 4-hydroxy-2-thiophen-2-ylpyrimidine-6-carboxylic acid as a white solid (0.81 g, 60%). ¹H nmr (400 MHz, d₆-DMSO) δ_{H} 8.1 (1H, m), 7.85 (1H, m), 7.2 (1H, m), 6.7 (1H, s) ppm (exchangeable protons not observed).

### 9.2 Preparation of 4-chloro-2-thiophen-2-ylpyrimidine-6-carboxylic acid

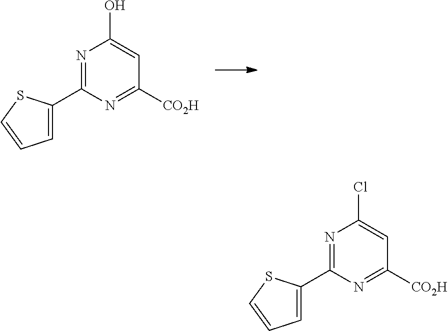

Phosphorus oxychloride (5 ml) was added to 4-hydroxy-2-thiophen-2-ylpyrimidine-6-carboxylic acid (0.81 g, 3.6 mmol) and the resulting suspension was heated at 90° C. for 1 hour, then allowed to cool and added dropwise to a stirred solution of acetonitrile:water (1:2, 50 ml), keeping the temperature below 50° C. The resulting solution was allowed to cool to ambient temperature and the precipitate isolated by filtration, washed with water and dried to yield 4-chloro-2-thiophen-2-ylpyrimidine-6-carboxylic acid (0.8 g, 90%).

### 9.3 Preparation of 4-amino-2-thiophen-2-ylpyrimidine-6-carboxylic acid

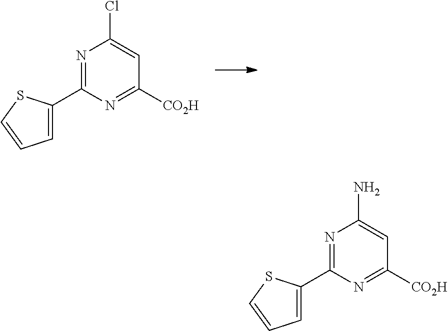

4-Chloro-2-thiophen-2-ylpyrimidine-6-carboxylic acid (0.8 g, 3.3 mmol) was suspended in aqueous ammonia (5%, 15 ml) and the mixture heated at 90° C. overnight. Further aqueous ammonia (5%, 1 ml) was added and heating continued for a further 24 hours. The mixture was cooled, concentrated under reduced pressure to approximately 10 ml volume and the precipitate isolated by filtration, washed with cold water and dried in vacuo to yield 4-amino-2-thiophen-2-ylpyrimidine-6-carboxylic acid as ¹H nmr (400 MHz, d₆-DMSO) δ_{H} 7.75 (1H, m), 7.55 (1H, m), 7.1 (1H, m), 6.85 (2H, br s), 6.65 (1H, s) ppm (acid proton not observed).

### Example 10

### Synthesis of 4-amino-5-chloro-2-(5-chlorothiophen-2-yl)-pyrimidine-6-carboxylic acid (compound 1-2)

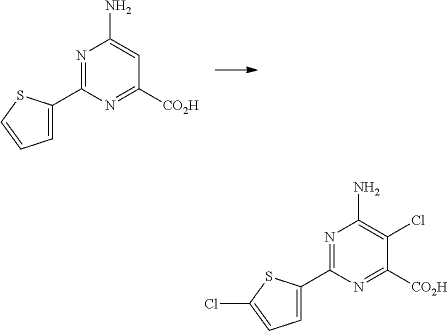

N-Chlorosuccinimide (0.26 g, 1.9 mmol) was added in three portions over 30 minutes to a stirred suspension of 4-amino-2-thiophen-2-ylpyrimidine-6-carboxylic acid (prepared as described in example 9) (0.4 g, 1.8 mmol) in dimethylformamide (5 ml). The reaction mixture was stirred at 50° C. for 3 hours, then cooled, diluted with water (100 ml) and extracted with ethyl acetate (3×100 ml). The combined organic extracts were dried over magnesium sulphate, filtered and evaporated under reduced pressure and the residue purified by preparative HPLC to give 4-amino-5-chloro-2-(5-chlorothiophen-2-yl)-pyrimidine-6-carboxylic acid (0.23 g, 44%). ¹H nmr (400 MHz, d₆-DMSO) δ_{H} 7.7 (1H, d) 7.1, (1H, d) ppm (exchangeable protons not observed).

### Example 11

### Synthesis of 4-amino-5-chloro-2-(5-chlorothiophen-2-yl)-6-ethoxycarbonyl-pyrimidine (compound 3-2)

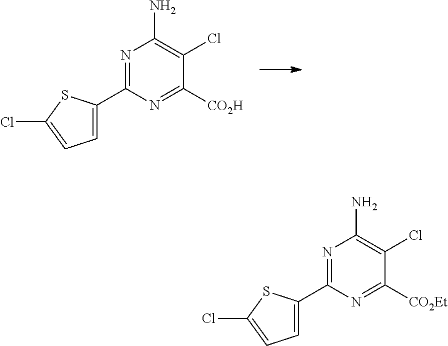

Thionyl chloride (0.1 ml) was added dropwise over 10 minutes to a stirred suspension of 4-amino-5-chloro-2-(5-chlorothiophen-2-yl)-pyrimidine-6-carboxylic acid (prepared as described in example 10) (0.15 g, 0.52 mmol) in ethanol (10 ml) and the mixture stirred for 3 hours. The reaction mixture was concentrated under reduced pressure, the residue dissolved in 1M aqueous sodium carbonate (50 ml) and the solution extracted with ethyl acetate (3×50 ml). The combined organic extracts were dried over magnesium sulphate, filtered and evaporated under reduced pressure to yield 4-amino-5-chloro-2-(5-chlorothiophen-2-yl)-6-ethoxycarbonylpyrimidine as a white solid (0.157 g, 95%). ¹H nmr (400 MHz, CDCl₃) δ_{H} 7.7 (1H, s), 6.9 (1H, s), 5.6 (2H, br s), 4.5 (2H, q), 1.5 (3H, t) ppm.

### Example 12

### Synthesis of 4-amino-5-chloro-6-ethoxycarbonyl-2-(5-trifluoromethyl-pyridin-2-yl)-pyrimidine (compound 3-150)

### 12.1 Preparation of 4-amino-6-ethoxycarbonyl-2-(5-trifluoromethylpyridin-2-yl)-pyrimidine

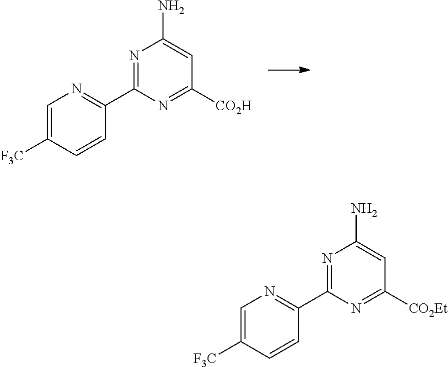

Thionyl chloride (0.1 ml) was added dropwise to a stirred solution of 4-amino-2-(5-trifluoromethylpyridin-2-yl)-pyrimidine-6-carboxylic acid (prepared by the method described in example 9) (0.3 g, 1.06 mmol) and the mixture stirred at ambient temperature for 3 hours. The solvent was evaporated under reduced pressure and the residue dissolved in 1M aqueous sodium carbonate (50 ml). The mixture was extracted with ethyl acetate (3×100 ml) and the combined organic extracts dried over magnesium sulphate, filtered and evaporated under reduced pressure to provide 4-amino-6-ethoxycarbonyl-2-(5-trifluoromethylpyridin-2-yl)-pyrimidine as a white solid.

### 12.2 Preparation of 4-amino-5-chloro-6-ethoxycarbonyl-2-(5-trifluoromethylpyridin-2-yl)-pyrimidine (compound 3-150)

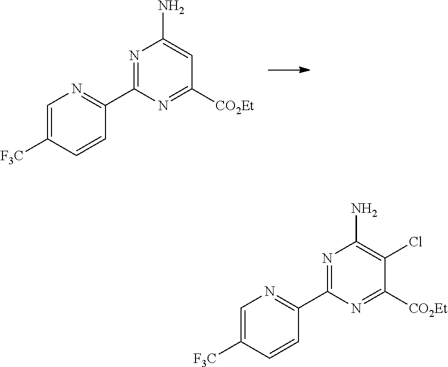

N-Chlorosuccinimide (0.103 g, 0.75 mmol) was added in three portions over 30 minutes to a stirred suspension of 4-amino-6-ethoxycarbonyl-2-(5-trifluoromethylpyridin-2-yl)-pyrimidine (0.2 g, 0.64 mmol) in dimethylformamide (5 ml). The reaction mixture was heated at 50° C. for 3 hours, then cooled and dissolved in ethyl acetate (25 ml). The solution was washed with water (3×50 ml) and brine (2×100 ml), dried over magnesium sulphate, filtered and evaporated under reduced pressure to leave a white solid which was purified by preparative HPLC on silica to provide 4-amino-5-chloro-6-ethoxycarbonyl-2-(5-trifluoromethylpyridin-2-yl)-pyrimidine (15 mg, 13%). M/z (LCMS) M+H⁺347, 349.

### Example 13

### Synthesis of 4,5-dichloro-2-pyridin-2-ylpyrimidine-6-carboxylic acid (compound 61-149)

### 13.1 Preparation of 4-hydroxy-2-pyridin-2-ylpyrimidine-6-carboxylic acid

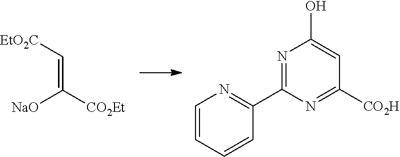

A solution of sodium hydroxide (2.17 g, 54 mmol) in water (4.5 ml) was added to a stirred suspension of diethyl oxaloacetate sodium salt (6.65 g, 31.7 mmol) in water (40 ml) and the mixture stirred for 20 minutes until all the solid dissolved. 2-Pyridineamidine (3.85 g, 31.8 mmol) was added and the reaction mixture stirred at 70° C. overnight, then allowed to cool. Concentrated hydrochloric acid was added to bring the mixture to pH 1 and the solid removed by filtration to give 4-hydroxy-2-pyridin-2-ylpyrimidine-6-carboxylic acid as an off-white solid (4.00 g, 58%). ¹H nmr (400 MHz, d₆-DMSO) δ_{H} 8.79 (1H, m), 8.42 (1H, d), 8.12 (1H, t), 7.70 (1H, m), 7.0-5.5 (2H, br s), 6.96 (1H, s) ppm.

### 13.2 Preparation of 5-chloro-4-hydroxy-2-pyridin-2-ylpyrimidine-6-carboxylic acid

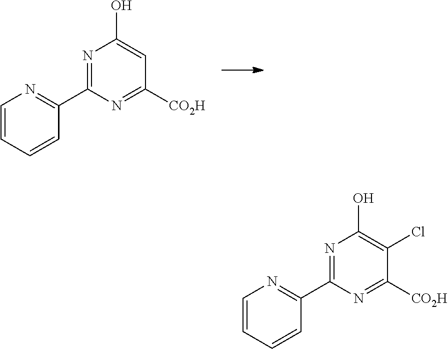

Aqueous sodium hypochlorite (11.3 ml) was added, with cooling, to a stirred suspension of 4-hydroxy-2-pyridin-2-ylpyrimidine-6-carboxylic acid (2.00 g, 9.2 mmol) in a mixture of concentrated hydrochloric acid (6.6 ml) and water (10 ml). The reaction mixture was stirred at ambient temperature for 4 hours, then sodium metabisulphite (0.66 g) and a solution of sodium hydroxide (2.82 g) in water (5.6 ml) was added with cooling. The resulting precipitate was filtered off and washed with ice cold water. This material was then suspended in concentrated hydrochloric acid (3.3 ml) and water (5 ml). Aqueous sodium hypochlorite (5.65 ml) was added and the mixture stirred at ambient temperature for 7 hours. Sodium metabisulphite (0.33 g) and a solution of sodium hydroxide (1.41 g) in water (2.8 ml) were added. The resulting precipitate was removed by filtration, washed with ice cold water and dried to provide 5-choro-4-hydroxy-2-pyridin-2-ylpyrimidine-6-carboxylic acid (1.80 g, 78%). ¹H nmr (400 MHz, d₆-DMSO) δ_{H} 8.78 (1H, m), 8.31 (1H, d), 8.06 (1H, t), 7.69 (1H, m), 4.0-3.0 (2H, br s) ppm.

### 13.3 Preparation of 4,5-dichloro-2-pyridin-2-ylpyrimidine-6-carboxylic acid (compound 61-149)

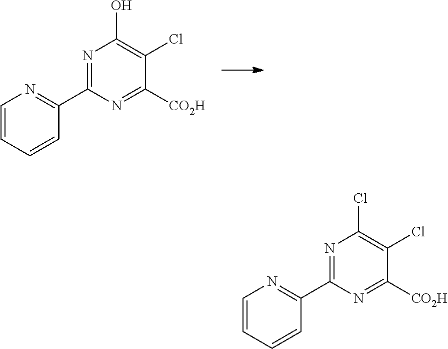

A mixture of 5-choro-4-hydroxy-2-pyridin-2-ylpyrimidine-6-carboxylic acid (1.2 g, 4.8 mmol) and phosphorus oxychloride (2.5 ml) was heated at reflux for 5.5 hours. The mixture was allowed to cool and then poured into a mixture of ice cold water (8 ml) and acetonitrile (4 ml), maintaining the temperature below 10° C. Aqueous ammonia was added to maintain the solution at pH 1-2. The resulting mixture was concentrated under reduced pressure to provide an orange solid that was washed with ice cold water to give 4,5-dichloro-2-pyridin-2-ylpyrimidine-6-carboxylic acid (0.60 g, 47%). ¹H nmr (400 MHz, d₆-DMSO) δ_{H} 8.84 (1H, m), 8.47 (1H, d), 8.19 (1H, t), 8.0-6.5 (1H, br s), 7.75 (1H, m).

### Example 14

### Synthesis of 4-amino-5-chloro-2-pyridin-2-ylpyrimidine-6-carboxylic acid (compound 1-149)

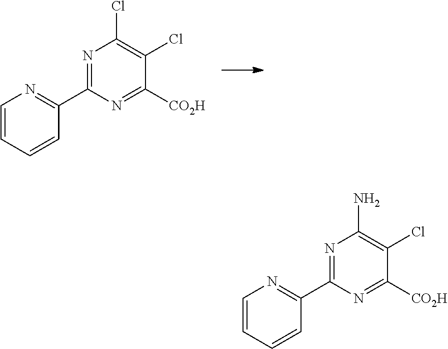

A suspension of 4,5-dichloro-2-pyridin-2-ylpyrimidine-4-carboxylic acid (prepared as described in example 13) (0.30 g, 1.1 mmol) in methanolic ammonia (7M, 2.5 ml) was heated in a sealed vial in a microwave reactor at 190° C. for 30 minutes. The reaction mixture was allowed to cool, 2N hydrochloric acid added until all the solid dissolved and the solvent evaporated under reduced pressure. The residue was washed with ice cold water to provide 4-amino-5-chloro-2-pyridin-2-ylpyrimidine-6-carboxylic acid as an orange solid (0.18 g, 64%). M.p. 183° C. (decomp.); ¹H nmr (400 MHz, d₆-DMSO) δ_{H} 8.65 (1H, m), 8.21 (1H, d), 7.90 (1H, t), 7.44 (1H, br s), 7.43 (1H, m), 7.15 (2H, br s) ppm.

### Example 15

### Alternative Synthesis for 4-amino-5-chloro-2-pyridin-2-ylpyrimidine-6-carboxylic acid (compound 1-149)

Aqueous ammonia (2.5 ml) was added to 4,5-dichloro-2-pyridin-2-ylpyrimidine-6-carboxylic acid (prepared as described in example 13) (0.28 g, 1.04 mmol), and the reaction mixture heated at 80° C. for 18 hours. The mixture was then cooled and concentrated under reduced pressure. The resulting solid was washed with ice cold water to provide 4-amino-5-chloro-2-pyridin-2-ylpyrimidine-4-carboxylic acid (0.15 g, 59%).

### Example 16

### Synthesis of 4-amino-5-chloro-6-ethoxycarbonyl-2-pyridin-2-ylpyrimidine (compound 3-149)

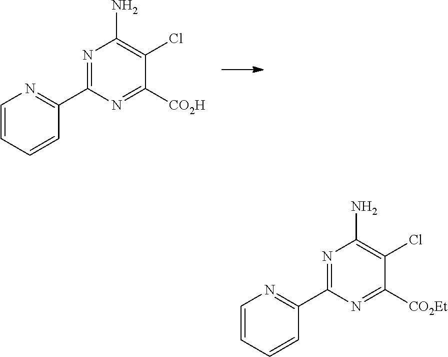

Thionyl chloride (0.04 ml) was added to a solution of 4-amino-5-chloro-2-pyridin-2-ylpyrimidine-6-carboxylic acid (prepared as described in example 15) (0.15 g, 0.60 mmol) in ethanol (1 ml) and the mixture heated at 70° C. for 18 hours. The resulting solution was allowed to cool, then neutralised by the addition of saturated aqueous sodium bicarbonate and extracted with ethyl acetate. The organic extracts were dried over magnesium sulphate, filtered and evaporated to provide 4-amino-5-chloro-6-ethoxycarbonyl-2-pyridin-2-ylpyrimidine as a pale orange solid (0.11 g, 68%). M.p. 176-180° C. (decomp.); ¹H nmr (400 MHz, CDCl₃) δ_{H} 8.80 (1H, m), 8.50 (1H, d), 7.85 (1H, t), 7.40 (1H, m), 5.85 (2H, br s), 4.50 (2H, q), 1.47 (3H, t) ppm.

### Example 17

### Pre-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retroflexus (AMARE) and Ipomea hederaceae (IPOHE) were sown in standard soil in pots. After cultivation for one day under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 250 or 1000 g/ha of test compound.

The test plants were then grown under controlled conditions in the glasshouse (at 24/16° C., day/night; 14 hours light; 65% humidity) and watered twice daily. After 13 days the test was evaluated (100=total damage to plant; 0=no damage to plant). Results are shown below in Table B1.

TABLE B1
Percentage damage caused to weed species by compounds of the invention when applied pre-emergence.

| | | Species | | | | | |
|---|---|---|---|---|---|---|---|
| Compound Number | Dose (g/ha) | SOLNI | AMARE | IPOHE | SETFA | ALOMY | ECHCG |
| 1-2 | 1,000 | 30 | 100 | 0 | 0 | 0 | 0 |
| 1-149 | 1,000 | 0 | 0 | 0 | 0 | 0 | 0 |
| 2-1 | 1,000 | 0 | 0 | 0 | 0 | 0 | 0 |
| 2-2 | 1,000 | 0 | 80 | 20 | 0 | 0 | 0 |
| 2-3 | 1,000 | 0 | 0 | 0 | 0 | 0 | 0 |
| 2-7 | 1,000 | 0 | 90 | 20 | 20 | 0 | 60 |
| 2-9 | 250 | 0 | 0 | 0 | 0 | 0 | 0 |
| 2-16 | 250 | 30 | 90 | 0 | 0 | 10 | 0 |
| 2-17 | 250 | 0 | 50 | 0 | 0 | 0 | 0 |
| 2-18 | 250 | 0 | 0 | 0 | 0 | 0 | 0 |
| 2-19 | 1,000 | 20 | 20 | 30 | 0 | 0 | 0 |
| 2-34 | 1,000 | 0 | 0 | 0 | 0 | 0 | 0 |
| 2-54 | 1,000 | 20 | 70 | 0 | 20 | 20 | 70 |
| 2-55 | 250 | 0 | 10 | 10 | 0 | 0 | 0 |
| 2-56 | 1,000 | 0 | 60 | 0 | 0 | 0 | 0 |
| 2-57 | 1,000 | 0 | 0 | 0 | 0 | 0 | 0 |
| 2-71 | 1,000 | 0 | 0 | 0 | 0 | 0 | 0 |
| 2-72 | 250 | 0 | 0 | 0 | 0 | 0 | 0 |
| 2-146 | 250 | 0 | 0 | 0 | 0 | 0 | 0 |
| 2-153 | 1,000 | 0 | 0 | 0 | 0 | 0 | 0 |
| 2-154 | 1,000 | 20 | 0 | 0 | 0 | 0 | 0 |
| 2-155 | 1,000 | 20 | 20 | 0 | 0 | 0 | 0 |
| 2-156 | 1,000 | 60 | 70 | 0 | 0 | 0 | 0 |
| 2-157 | 1,000 | 0 | 0 | 0 | 0 | 0 | 0 |
| 2-158 | 1,000 | 0 | 0 | 0 | 0 | 0 | 0 |
| 2-159 | 1,000 | 0 | 0 | 0 | 0 | 0 | 0 |
| 2-161 | 1,000 | 40 | 60 | 0 | 0 | 0 | 0 |
| 2-166 | 250 | 0 | 0 | 0 | 0 | 0 | 0 |
| 2-172 | 250 | 0 | 0 | 0 | 0 | 0 | 0 |
| 2-179 | 1,000 | 0 | 0 | 0 | 0 | 0 | 0 |
| 2-182 | 1,000 | 40 | 0 | 0 | 0 | 0 | 0 |
| 2-196 | 1,000 | 0 | 0 | 0 | 0 | 0 | 0 |
| 3-2 | 1,000 | 0 | 30 | 0 | 0 | 0 | 0 |
| 3-149 | 1,000 | 0 | 0 | 0 | 0 | 0 | 0 |
| 3-150 | 1,000 | 0 | 0 | 0 | 0 | 0 | 0 |
| 61-149 | 1,000 | 0 | 0 | 0 | 0 | 0 | 0 |
| 68-156 | 1,000 | 40 | 100 | 40 | 10 | 10 | 0 |

### Example 18

### Post-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retroflexus (AMARE) and Ipomea hederaceae (IPOHE) were sown in standard soil in pots. After cultivation for 8 days under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 250 or 1000 g/ha of test compound.

The test plants were then grown on under controlled conditions in a glasshouse (at 24/16° C., day/night; 14 hours light; 65% humidity) and watered twice daily. After 13 days the test was evaluated (100=total damage to plant; 0=no damage to plant). Results are shown below in Table B2.

TABLE B2
Percentage damage caused to weed species by compounds of the invention when applied post-emergence

| | | Species | | | | | |
|---|---|---|---|---|---|---|---|
| Compound Number | Dose (g/ha) | SOLNI | AMARE | IPONE | SETFA | ALOMY | ECHCG |
| 1-2 | 1,000 | 80 | 100 | 60 | 50 | 10 | 20 |
| 1-149 | 1,000 | 0 | 0 | 0 | 0 | 0 | 0 |
| 2-1 | 1,000 | 0 | 0 | 50 | 0 | 0 | 0 |
| 2-2 | 1,000 | 90 | 100 | 70 | 0 | 0 | 0 |
| 2-3 | 1,000 | 0 | 0 | 0 | 0 | 0 | 0 |
| 2-7 | 1,000 | 80 | 100 | 80 | 70 | 0 | 80 |
| 2-9 | 250 | 0 | 0 | 0 | 0 | 0 | 0 |
| 2-16 | 250 | 0 | 0 | 40 | 0 | 20 | 0 |
| 2-17 | 250 | 0 | 0 | 0 | 0 | 0 | 0 |
| 2-18 | 250 | 0 | 20 | 40 | 10 | 10 | 10 |
| 2-19 | 1,000 | 70 | 100 | 70 | 30 | 0 | 10 |
| 2-34 | 1,000 | 10 | 30 | 50 | 0 | 10 | 0 |
| 2-54 | 1,000 | 40 | 70 | 80 | 50 | 0 | 70 |
| 2-55 | 250 | 30 | 70 | 40 | 0 | 10 | 0 |
| 2-56 | 1,000 | 40 | 100 | 20 | 10 | 20 | 40 |
| 2-57 | 1,000 | 0 | 0 | 20 | 0 | 0 | 0 |
| 2-71 | 1,000 | 0 | 0 | 30 | 0 | 0 | 0 |
| 2-72 | 250 | 0 | 0 | 0 | 0 | 0 | 0 |
| 2-146 | 250 | 30 | 30 | 40 | 0 | 0 | 0 |
| 2-153 | 1,000 | 0 | 0 | 0 | 0 | 0 | 0 |
| 2-154 | 1,000 | 30 | 70 | 20 | 0 | 0 | 0 |
| 2-155 | 1,000 | 70 | 100 | 70 | 80 | 0 | 80 |
| 2-156 | 1,000 | 80 | 100 | 70 | 40 | 40 | 30 |
| 2-157 | 1,000 | 60 | 60 | 70 | 20 | 30 | 0 |
| 2-158 | 1,000 | 20 | 0 | 20 | 0 | 0 | 0 |
| 2-159 | 1,000 | 50 | 100 | 10 | 30 | 0 | 10 |
| 2-161 | 1,000 | 80 | 90 | 100 | 80 | 30 | 60 |
| 2-166 | 250 | 0 | 0 | 0 | 0 | 0 | 0 |
| 2-172 | 250 | 60 | 0 | 10 | 10 | 10 | 0 |
| 2-179 | 1,000 | 30 | 30 | 40 | 10 | 0 | 0 |
| 2-182 | 1,000 | 70 | 20 | 30 | 10 | 0 | 0 |
| 2-196 | 1,000 | 0 | 0 | 0 | 0 | 0 | 0 |
| 3-2 | 1,000 | 70 | 80 | 50 | 0 | 0 | 0 |
| 3-149 | 1,000 | 0 | 0 | 0 | 0 | 0 | 0 |
| 3-150 | 1,000 | 0 | 0 | 0 | 0 | 0 | 0 |
| 61-149 | 1,000 | 40 | 0 | 0 | 0 | 0 | 0 |
| 68-156 | 1,000 | 90 | 100 | 50 | 0 | 0 | 0 |

1. A method of controlling undesired plant growth, which comprises applying to said undesired plants a compound of formula (I) 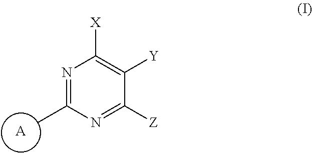
or salt or N-oxide thereof,
wherein:
A is a 5- or 6-membered heteroaromatic ring containing 1-4 heteroatoms and optionally substituted by 1-4 groups R¹, wherein said heteroatom(s) is (are) selected from O, N and S provided said heteroaromatic ring contains only one O or one S atom;
each R¹ is independently: halogen, cyano, nitro, azido, hydroxy, alkyl optionally substituted by one or more R^{a}, alkenyl optionally substituted by one or more R^{b}, alkynyl optionally substituted by one or more R^{c}, cycloalkyl optionally substituted by one or more R^{d}, OR^{aa}, S(O)ₐR^{bb}, C(O)R^{cc}, NR^{dd}R^{ee}, SiR^{ff}R^{gg}R^{hh}, (P(O)RⁱⁱR^{jj}, or B(OR^{kk})(OR^{LL}); or 2 adjacent R¹ groups together with the atoms to which they are joined form a 5-7 membered ring, said ring optionally containing 1 or 2 heteroatoms selected from O, S and N, and being optionally substituted with 1-4 groups R³;
each R³ is independently: halogen; cyano; nitro; hydroxy; alkyl optionally substituted by one or more R^{a}; alkenyl optionally substituted by one or more R^{b}; alkynyl optionally substituted by one or more R^{c}; cycloalkyl optionally substituted by one or more R^{d}; OR^{aa}; S(O)ₐR^{bb}; C(O)R^{cc}; NR^{dd}R^{ee}; SiR^{ff}R^{gg}R^{hh}; P(O)RⁱⁱR^{jj}; B(OR^{kk})(OR^{LL}); or any two geminal groups R³ together form a group selected from: oxo; ═CR^{mm}Rⁿⁿ, ═NOR^{oo}, and ═NNR^{pp}R^{qq};
each R^{a} is independently: halogen, cyano, nitro, hydroxy, cycloalkyl, OR^{aa}, S(O)ₐR^{bb}, C(O)R^{cc}, or NR^{dd}R^{ee};
each R^{b} is independently: halogen, cyano, nitro, hydroxy, alkoxy, S(O)₂R^{bb}, C(O)R^{cc}, or P(O)RⁱⁱR^{jj};
each R^{c} is independently: halogen, cyano, alkoxy, S(O)₂R^{bb}, C(O)R^{cc}, or SiR^{ff}R^{gg}R^{hh};
each R^{d} is independently: halogen, cyano, nitro, hydroxy, alkyl, alkenyl, alkynyl, cycloalkyl, alkoxy, S(O)ₐR^{bb}, or C(O)R^{cc};
each R^{aa} is independently: alkyl, haloalkyl, alkoxyalkyl, cycloalkylalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, dialkylaminocarbonyl, alkylimino, or dialkylimino;
a is an integer selected from 0, 1 and 2;
each R^{bb} is independently: alkyl, haloalkyl, alkoxyalkyl, alkenyl, haloalkenyl, alkynyl, haloalkynyl, cycloalkyl, or alkylcarbonylamino;
R^{cc} is: hydrogen, hydroxy, alkyl, haloalkyl, alkoxyalkyl, alkenyl, haloalkenyl, alkynyl, haloalkynyl, cycloalkyl, alkoxy, haloalkoxy, alkoxyalkoxy, arylalkoxy, cycloalkoxy, amino, alkylamino, dialkylamino, or alkylsulphonylamino;
R^{dd} is: hydrogen, alkyl, haloalkyl, alkoxyalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl;
R^{ee} is: hydrogen, alkyl, haloalkyl, alkoxyalkyl, alkenyl, haloalkenyl, alkynyl, haloalkynyl, or cycloalkyl;
or R^{dd} and R^{ee}, together with the N atom to which they are joined form a 4-6 membered ring, optionally containing one further heteroatom selected from O, N and S, said ring being optionally substituted by 1-4 groups selected from: halogen, alkyl, alkoxy, alkylcarbonyl, alkoxycarbonyl, and oxo;
R^{ff}, R^{gg}, and R^{hh} are each independently alkyl or haloalkyl;
Rⁱⁱ is alkyl, haloalkyl, alkoxy, or haloalkoxy;
R^{jj} is alkoxy or haloalkoxy;
R^{kk} and R^{LL} are each independently hydrogen or alkyl; or R^{kk} and R^{LL} together with the O atoms and B atom to which they are joined form a 5- or 6-membered heterocyclic ring optionally substituted by 1-4 alkyl groups;
R^{mm} is: hydrogen, halogen, cyano, nitro, alkyl, haloalkyl, alkoxyalkyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl;
Rⁿⁿ is: hydrogen, halogen, cyano, nitro, alkyl, haloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl or dialkylaminocarbonyl;
R^{oo} is: hydrogen, alkyl, haloalkyl, alkoxyalkyl, cycloalkyl, alkylcarbonyl, haloalkylcarbonyl, alkoxycarbonyl, haloalkoxycarbonyl, aminocarbonyl, alkylaminocarbonyl, or dialkylaminocarbonyl;
R^{pp} is: hydrogen, alkyl, haloalkyl, alkoxyalkyl, or cycloalkyl;
R^{qq} is: hydrogen, alkyl, haloalkyl, alkylcarbonyl, haloalkylcarbonyl, alkoxycarbonyl, aminocarbonyl, alkylaminocarbonyl, or dialkylaminocarbonyl; or R^{pp} and R^{qq} together with the nitrogen to which they are attached form a 5- or 6-membered optionally substituted ring, said ring optionally containing a further heteroatom selected from O, S, and N, said substitution(s) being selected from halogen and alkyl;
X is nitro, azido, halogen, optionally substituted alkoxy, optionally substituted aminoxy, or NR⁵R⁶, wherein:
R⁵ is: hydrogen, optionally substituted alkyl provided said substitution does not comprise a ring system, formyl, optionally substituted alkylcarbonyl provided said substitution does not comprise an aryl moiety, optionally substituted cycloalkylcarbonyl, optionally substituted alkenylcarbonyl, optionally substituted alkynylcarbonyl, optionally substituted phenylcarbonyl, optionally substituted heteroarylcarbonyl, optionally substituted aminocarbonyl, optionally substituted alkoxycarbonyl, optionally substituted alkylsulphonyl, optionally substituted cycloalkylsulphonyl, optionally substituted alkenylsulphonyl, optionally substituted alkynylsulphonyl, optionally substituted phenylsulphonyl, optionally substituted amino, hydroxy, optionally substituted alkoxy, optionally substituted cycloalkoxy, optionally substituted alkylcarbonyloxy, optionally substituted alkoxycarbonyloxy, optionally substituted aminocarbonyloxy, optionally substituted alkenyl provided said substitution does not comprise a ring system, optionally substituted alkynyl provided said substitution does not comprise a ring system, N═CR^{g}R^{h}, cyano, optionally substituted silyl, or optionally substituted phosphonyl;
and R⁶ is: hydrogen, optionally substituted alkyl provided said substitution does not comprise a ring system, optionally substituted alkenyl provided said substitution does not comprise a ring system, or optionally substituted alkynyl provided said substitution does not comprise a ring system; or R⁵ and R⁶ together form a group ═C(Rⁱ)OR^{j}, ═C(R^{k})SR^{L}, or ═C(R^{m})NRⁿR^{o}; or R⁵ and R⁶ together with the N atom to which they are attached form a 3-8 membered optionally substituted ring system, said ring system optionally containing 1-3 further heteroatoms independently selected from O, S and N;
R^{g} is: hydrogen, optionally substituted alkyl, optionally substituted cycloalkyl, optionally substituted phenyl, optionally substituted heteroaryl, optionally substituted amino, optionally substituted alkoxy, optionally substituted phenoxy, optionally substituted alkylthio, or optionally substituted phenylthio;
R^{h} is: optionally substituted alkyl, optionally substituted cycloalkyl, optionally substituted phenyl, optionally substituted heteroaryl, optionally substituted amino, optionally substituted alkoxy, or optionally substituted alkylthio; or R^{g} and R^{h} together with the C atom to which they are joined form an optionally substituted 5-7 membered ring system;
Rⁱ is hydrogen, optionally substituted alkyl provided said substitution does not comprise a ring system, optionally substituted amino, optionally substituted alkoxy, or optionally substituted alkylthio;
R^{j} is optionally substituted alkyl or optionally substituted cycloalkyl;
R^{k} is hydrogen, optionally substituted alkyl provided said substitution does not comprise a ring system, optionally substituted amino, or optionally substituted alkylthio;
R^{L} is optionally substituted alkyl, or optionally substituted cycloalkyl;
R^{m} is hydrogen, optionally substituted alkyl, optionally substituted cycloalkyl, optionally substituted phenyl, optionally substituted heteroaryl, or optionally substituted amino;
Rⁿ and R^{o} are each independently hydrogen, optionally substituted alkyl, or optionally substituted cycloalkyl;
Y is: halogen, cyano, optionally substituted alkyl, optionally substituted cycloalkyl, optionally substituted alkenyl, optionally substituted alkynyl, optionally substituted phenyl, optionally substituted heteroaryl, optionally substituted alkoxy, optionally substituted amino, optionally substituted alkylthio, optionally substituted alkylsulphinyl, optionally substituted alkylsulphonyl, optionally substituted dialkyl phosphonyl, or optionally substituted trialkylsilyl;
Z is: (T)ₘ-(U)ₙ—V; wherein,
m is an integer of 0 or 1; n is an integer selected from 0, 1, 2 and 3, and n≧m;
T is an oxygen or sulphur atom;
U is CR^{w}R^{x};
each R^{w} is independently hydrogen, halogen, hydroxy, optionally substituted alkyl, optionally substituted alkoxycarbonyl, or OR^{y} and each R^{y} is independently an optionally substituted alkyl or an optionally substituted alkylcarbonyl;
each R^{x} is independently hydrogen, halogen, optionally substituted alkyl, or OR^{Z} and each R^{Z} is independently an optionally substituted alkyl;
or any geminal R^{w} and R^{x} together form a group selected from oxo, or ═NOR^{ca}, wherein R^{ca} is hydrogen or optionally substituted alkyl; or any geminal, vicinal or non-adjacent R^{w} and/or R^{x} together with the C atom(s) to which they are attached and any intervening atom form an optionally substituted 3-6 membered ring;
or wherein when at least one R^{w} is OR^{y} and at least one R^{x} is OR^{Z}, said OR^{y} and OR^{Z} groups together with the C atom(s) to which they are attached and any intervening atom form an optionally substituted 5-6 membered heterocyclic ring;
V is C(O)R^{cb}, C(S)R^{cd}, C(═NR^{ce})R^{cf}, CHR^{cg}R^{ch}, CH(S[O]ₚR^{ci})(S[O]_{q}R^{cj}), CR^{ck}R^{cl}R^{cm}, or CH₂OR^{cn};
R^{cb} is hydrogen, hydroxy, optionally substituted alkoxy, optionally substituted cycloalkoxy, optionally substituted alkylthio, or optionally substituted amino;
R^{cd} is optionally substituted alkoxy, optionally substituted cycloalkoxy, optionally substituted alkylthio, or optionally substituted amino;
R^{ce} is hydrogen, optionally substituted alkyl, optionally substituted alkoxy, optionally substituted cycloalkoxy, or optionally substituted amino;
R^{cf} is hydrogen, optionally substituted alkoxy, optionally substituted cycloalkoxy, optionally substituted alkylthio, or optionally substituted amino;
R^{cg} and R^{ch} are each independently an optionally substituted alkoxy group; or R^{cg} and R^{ch}, together with the carbon to which they are joined, form a dioxolane or dioxane ring, which ring is optionally substituted;
R^{ci} and R^{cj} are each independently an optionally substituted alkyl group; or R^{ci} and R^{cj}, together with the carbon and sulphur atoms to which they are joined, form an optionally substituted 5-6 membered ring;
p and q are each independently an integer of 0, 1, or 2
R^{ck}, R^{cl}, and R^{cm} are each independently an optionally substituted alkoxy group; or R^{ck} and R^{cl} and R^{cm} together with the carbon to which they are attached form an optionally substituted trioxabicyclo[2.2.2]octane ring system;
and R^{cn} is hydrogen or an optionally substituted alkylcarbonyl group.

2. The method according to claim 1, wherein:
A is a ring system selected from:
thiophene, furan, pyrrole, isoxazole, isothiazole, oxazole, thiazole, imidazole, pyrazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,3,4-oxatriazole, 1,2,3,4-thiatriazole, tetrazole, pyridine, pyridazine, pyrimidine, pyrazine, 1,2,3-triazine, 1,2,4-triazine, 1,3,5-triazine, and 1,2,4,5-tetrazine, wherein said ring system is optionally substituted by 1-3 groups R¹;
each R¹ is independently: halogen; cyano; nitro; hydroxy; C₁₋₆ alkyl optionally substituted by 1-4 groups R^{a}; C₁₋₆ haloalkyl optionally substituted by 1-4 groups R^{a}; OR^{aa}; S(O)ₐR^{bb}; C(O)R^{cc}; NR^{dd}R^{ee}; or 2 adjacent groups R¹ together with the atoms to which they are joined form a 5-6 membered ring, optionally containing 1 or 2 heteroatoms selected from O, N and S, said 5-6 membered ring being optionally substituted with 1-4 groups R³;
each R³ is independently: halogen; cyano; nitro; hydroxy; C₁₋₆alkyl optionally substituted by 1-4 groups R^{a}; C₁₋₆haloalkyl optionally substituted by 1-4 groups R^{a}; OR^{aa}; S(O)ₐR^{bb}; C(O)R^{cc}; NR^{dd}R^{ee}; or any two geminal groups R³ together form a group selected from: oxo, ═CR^{mm}Rⁿⁿ, ═NOR^{oo}, and ═NNR^{pp}R^{qq};
each R^{a} is independently: cyano, hydroxy, C₃₋₆ cycloalkyl, OR^{aa}, S(O)ₐR^{bb}, C(O)R^{cc}, or NR^{dd}R^{ee};
each R^{aa} is independently: C₁₋₆ alkyl, C₁₋₆ haloalkyl, C₂₋₈ alkoxyalkyl, or C₁₋₆ alkylcarbonyl;
each R^{bb} is independently: C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino;
a is as defined in claim 1;
R^{cc} is hydrogen, hydroxy, C₁₋₆ alkyl, C₁₋₆ haloalkyl, C₃₋₆ cycloalkyl, C₁₋₆ alkoxy, phenylC₁₋₆alkoxy, C₃₋₆ cycloalkoxy, amino, C₁₋₆ alkylamino, C₂₋₈ dialkylamino, or C₁₋₆ alkylsulphonylamino;
R^{dd} is hydrogen, C₁₋₆ alkyl, C₁₋₆ alkylcarbonyl, C₂₋₆ alkoxycarbonyl, C₁₋₆ alkylsulphonyl, C₁₋₆ haloalkylsulphonyl, aminocarbonyl, C₁₋₆ alkylaminocarbonyl, or C₂₋₈ dialkylaminocarbonyl;
R^{ee} is hydrogen, C₁₋₆ alkyl, or C₃₋₆ cycloalkyl;
or R^{dd} and R^{ee}, together with the N atom to which they are joined form a 5 or 6 membered saturated ring, optionally containing one further heteroatom selected from O, N and S, said ring being optionally substituted by 1-2 groups selected from C₁₋₆ alkyl and oxo;
R^{mm} is hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl;
Rⁿⁿ is hydrogen, halogen, cyano, nitro, C₁₋₆ alkyl, C₁₋₆ alkylcarbonyl, C₁₋₆ alkoxycarbonyl, C₁₋₆ alkylsulphonyl, or aminocarbonyl;
R^{oo} and R^{pp} are each independently: hydrogen, C₁₋₆ alkyl, or C₃₋₆ cycloalkyl;
R^{qq} is hydrogen, or C₁₋₆ alkyl;
X is: NR⁵R⁶, wherein
R⁵ is: hydrogen; alkyl optionally substituted by one or more R^{p}; alkenyl optionally substituted by one or more R^{t}; alkynyl optionally substituted by one or more groups R^{u}; OR^{rr}; SO₂R^{ss}; C(O)R^{uu}; NR^{vv}R^{ww}; N═CR^{g}R^{h}; cyano; trialkylsilyl; or dialkyl phosphonyl;
R⁶ is: hydrogen, alkyl optionally substituted by one or more R^{p}, alkenyl optionally substituted by one or more R^{t}, or alkynyl optionally substituted by one or more groups R^{u}; or R⁵ and R⁶ together form a group ═C(Rⁱ)OR^{j}, ═C(R^{k})SR^{L}, ═C(R^{m})NRⁿR^{o}; or R⁵ and R⁶ together with the N atom to which they are attached form a 3-8 membered ring system, said ring system optionally containing 1-3 further heteroatoms independently selected from O, N and S and said ring system being optionally substituted by 1-4 groups R^{v};
R^{g} and R^{h} are each independently: hydrogen; alkyl optionally substituted by one or more R^{xx}; cycloalkyl substituted by one of more R^{yy}; phenyl substituted by one or more R^{zz}; heteroaryl substituted by one of more R^{ab}; NR^{ac}R^{ad}; OR^{ae}; or SR^{af}; or R^{g} and R^{h} together with the C atom to which they are joined form a 5-7 membered ring system optionally substituted by 1-4 groups R^{ag};
Rⁱ is hydrogen; alkyl optionally substituted by one or more R^{xx}; NR^{ac}R^{ad}; OR^{ae}; or SR^{af};
R¹ is alkyl optionally substituted by one or more R^{ah} or cycloalkyl substituted by one of more R^{ai};
R^{k} is hydrogen; alkyl optionally substituted by one or more R^{xx}; NR^{ac}R^{ad}; or SR^{af};
R^{L} is alkyl optionally substituted by one or more R^{ah} or cycloalkyl optionally substituted by one or more R^{ai};
R^{m} is: hydrogen; alkyl optionally substituted by one or more R^{xx}; cycloalkyl optionally substituted by one or more R^{yy}; phenyl optionally substituted by one or more R^{zz}; heteroaryl substituted by one or more R^{ab}; or NR^{ac}R^{ad};
Rⁿ and R^{o} are each independently hydrogen, alkyl optionally substituted by one or more R^{ah}, or cycloalkyl optionally substituted by one or more R^{ai};
R^{p} is halogen, cyano, nitro, hydroxy, alkoxy, alkoxyalkoxy, S(O)_{b}R^{am}, C(O)R^{an}, or NR^{ao}R^{ap};
R^{t} is halogen, cyano, or alkoxycarbonyl;
R^{u} is halogen, cyano, alkoxy, or alkoxycarbonyl;
each R^{v} is independently: halogen; cyano; nitro; hydroxy; alkyl optionally substituted by one or more R^{aq}; alkenyl optionally substituted by one or more R^{ar}; alkynyl optionally substituted by one or more R^{as}; cycloalkyl substituted by one or more R^{at}; OR^{al}; S(O)_{b}R^{am}; C(O)R^{an}; or NR^{au}R^{av}; or any two geminal groups R^{v} together form an oxo group;
each R^{rr} is hydrogen, alkyl, haloalkyl, cycloalkyl, alkylcarbonyl, haloalkylcarbonyl, alkoxycarbonyl, aminocarbonyl, alkylaminocarbonyl, dialkylaminocarbonyl, or phenyl optionally substituted by one or more groups R^{zz};
each R^{ss} is alkyl, haloalkyl, alkenyl, haloalkenyl, alkynyl, haloalkynyl, cycloalkyl, or phenyl optionally substituted by one or more groups R^{zz};
each R^{uu} is: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; NR^{ac}R^{ad}; or OR^{ae};
each R^{vv} is independently: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; SO₂R^{ss}; or C(O)R^{uu};
each R^{ww} is independently: hydrogen, alkyl optionally substituted by one or more R^{xx}; cycloalkyl optionally substituted by one or more R^{yy}; phenyl optionally substituted by one or more R^{zz}; or heteroaryl optionally substituted by one or more R^{ab}; or R^{vv} and R^{ww} together with the N atom to which they are attached form a 5-6 membered ring, said ring optionally containing one further heteroatom selected from O, N, and S and being optionally substituted by 1-2 groups selected from alkyl and alkylcarbonyl;
each R^{xx} is independently halogen, cyano, alkoxy, or alkoxycarbonyl;
each R^{yy} is independently: halogen; cyano; alkyl; cycloalkyl; phenyl optionally substituted by one or more R^{zz}; heteroaryl optionally substituted by one or more R^{ab}; or alkoxycarbonyl;
each R^{zz} is independently: halogen; cyano; nitro; hydroxy; alkyl optionally substituted by one or more R^{aq}; alkenyl optionally substituted by one or more R^{ar}, alkynyl optionally substituted by one or more R^{as}; OR^{al}, S(O)_{b}R^{am}; or C(O)R^{an};
each R^{ab} is independently: halogen; cyano; hydroxy; alkyl optionally substituted by one or more R^{aq}; alkenyl optionally substituted by one or more R^{ar}, alkynyl optionally substituted by one or more R^{as}; OR^{al}, S(O)_{b}R^{am}; or C(O)R^{an};
R^{ae} is hydrogen, alkyl, haloalkyl, alkoxyalkyl, alkylcarbonyl, alkoxycarbonyl, or phenyl optionally substituted by one or more R^{zz};
R^{ad} is hydrogen, alkyl, haloalkyl, alkoxyalkyl, or phenyl optionally substituted by one or more R^{zz};
R^{ae} is alkyl, phenylalkyl optionally substituted by one or more R^{zz}, or phenyl optionally substituted by one or more R^{zz};
R^{af} is alkyl or phenyl;
each R^{ag} is independently alkyl or haloalkyl;
each R^{ah} is independently halogen or phenyl;
each R^{ai} is independently halogen or alkyl;
R^{al} is alkyl, haloalkyl, alkoxyalkyl, or alkylsulphonyl;
R^{am} is alkyl, haloalkyl, alkenyl, alkynyl, phenyl, or alkylcarbonylamino;
b is an integer selected from 0, 1, and 2;
R^{an} is hydrogen, alkyl, haloalkyl, alkoxy, haloalkoxy, amino, alkylamino, dialkylamino, or alkylsulphonylamino;
R^{ao} is hydrogen, alkyl, alkoxyalkyl, phenylalkyl, formyl, alkylcarbonyl, alkoxycarbonyl, aminocarbonyl, alkylaminocarbonyl, dialkylaminocarbonyl;
R^{ap} is hydrogen, alkyl, alkoxyalkyl, or phenylalkyl;
each R^{aq} independently halogen, alkoxy, cyano, alkoxycarbonyl, or alkylsulphonyl;
each R^{ar} is independently halogen, cyano, nitro, alkoxy, alkylcarbonyl, alkoxycarbonyl, aminocarbonyl, alkylsulphonyl, or dialkyl phosphonyl;
each R^{as} is independently halogen, cyano, trialkylsilyl, alkylcarbonyl, alkoxycarbonyl, aminocarbonyl, or alkylsulphonyl;
each R^{at} is independently halogen, alkyl, or alkoxycarbonyl;
R^{au} is hydrogen, alkyl, haloalkyl, alkoxyalkyl, alkylcarbonyl, alkoxycarbonyl, aminocarbonyl, or phenyl optionally substituted by one or more R^{zz};
R^{av} is hydrogen, alkyl, haloalkyl, alkoxyalkyl, or phenyl optionally substituted by one or more R^{zz}; or R^{au} and R^{av} together with the N atom to which they are attached form a 5-6 membered ring, said ring optionally containing one further heteroatom selected from O, S, and N, and being optionally substituted by 1-2 groups selected from alkyl and alkylcarbonyl;
Y is: halogen; cyano; C₁₋₆ alkyl optionally substituted by 1-3 groups R^{ba}; C₁₋₆ haloalkyl optionally substituted by 1-3 groups R^{ba}; C₃₋₆ cycloalkyl optionally substituted by 1-3 groups R^{bc}; C₂₋₆ alkenyl optionally substituted by 1-3 groups R^{bd}; C₂₋₆ alkynyl optionally substituted by 1-3 groups R^{be}; phenyl optionally substituted by 1-4 groups R^{bf}; heteroaryl optionally substituted by 1-3 groups R^{bg}; OR^{bh}; NR^{bi}R^{bj}; S(O)_{c}R^{bk}; C₂₋₈dialkyl phosphonyl; or C₃₋₁₂-trialkylsilyl; wherein
each R^{ba} is independently cyano, nitro, hydroxy, C₁₋₄ alkoxy, C₁₋₄ alkylthio, C₁₋₄ alkylcarbonyl, or C₁₋₄ alkoxycarbonyl;
each R^{bc} is independently halogen, cyano, C₁₋₄ alkyl, C₁₋₄ alkoxy, or C₁₋₄ alkoxycarbonyl;
each R^{bd} is independently halogen, cyano, C₁₋₄ alkylcarbonyl, or C₁₋₄ alkoxycarbonyl;
each R^{be} is independently halogen, cyano, hydroxy, C₁₋₄ alkoxycarbonyl, or C₃₋₁₂ trialkylsilyl;
each R^{bf} and R^{bg} are independently halogen, cyano, C₁₋₄ alkyl, C₁₋₄ haloalkyl, C₂₋₈ alkoxyalkyl, C₁₋₄ alkoxy, C₁₋₄ haloalkoxy, C₁₋₄ alkylsulphonyl, or C₁₋₄ alkoxycarbonyl;
R^{bh} is C₁₋₄ alkyl, C₁₋₄ haloalkyl, or C₁₋₄ alkylsulphonyl;
R^{bi} is hydrogen, C₁₋₄ alkyl, C₁₋₄ alkylcarbonyl, C₂₋₄ alkoxycarbonyl, aminocarbonyl, C₁₋₄ alkylaminocarbonyl, or C₂₋₈ dialkylaminocarbonyl;
R^{bj} is hydrogen, or C₁₋₄ alkyl;
R^{bk} is C₁₋₄ alkyl, or C₁₋₄ haloalkyl;
c is an integer selected from 0, 1, and 2;
Z is (O)ₘ—(U)ₙ—V;
m and n are as defined in claim 1;
U is CR^{w}R^{x};
R^{w} is hydrogen, halogen, hydroxy, C₁₋₄ alkyl, C₁₋₄ haloalkyl, C₁₋₃ alkoxy(C₁₋₃)alkyl, C₁₋₆ alkoxycarbonyl, C₁₋₄ alkoxy, or C₁₋₄ alkylcarbonyloxy;
R^{x} is hydrogen, halogen, C₁₋₄ alkyl, C₁₋₄ haloalkyl, C₂₋₆alkoxyalkyl; C₁₋₄ alkoxy; or any geminal R^{w} and R^{x} together form a group selected from oxo, or ═NOR^{ca}, wherein R^{ca} is hydrogen or C₁₋₄ alkyl;
V is C(O)R^{cb}, C(S)R^{cd}, C(═NR^{ce})R^{cf}, CHR^{cg}R^{ch}, CH(S[O]ₚR^{ci})(S[O]_{q}R^{cj}), CR^{ck}R^{cl}R^{cm}, or CH₂OR^{cn};
R^{cb} is hydrogen, hydroxy, C₁₋₁₀ alkylthio, amino, C₁₋₆ alkylamino, di(C₁₋₄)alkylamino, or OR^{co};
R^{cd} is C₁₋₂₀ alkoxy, C₁₋₁₀ alkylthio, amino, C₁₋₆ alkylamino, or di(C₁₋₄)alkylamino;
R^{ce} is hydrogen, C₁₋₆ alkyl, amino, C₁₋₆ alkylamino, di(C₁₋₄)alkylamino, C₁₋₆ alkoxy, or C₃₋₆ cycloalkoxy;
R^{cf} is hydrogen, C₁₋₆ alkoxy, C₁₋₆ alkylthio, amino, C₁₋₆ alkylamino, or di(C₁₋₄)alkylamino;
R^{cg} and R^{ch} are each independently C₁₋₄ alkoxy; or R^{cg} and R^{ch} together with the carbon to which they are joined form a dioxolane or dioxane ring, which is optionally substituted by 1-2 C₁₋₂ alkyl groups;
R^{ci} and R^{cj} are each independently C₁₋₄ alkyl; or R^{ci} and R^{cj} together with the carbon and sulphur atoms to which they are joined form a 5-6 membered ring optionally substituted by 1-2 C₁₋₂ alkyl groups;
p and q are independently either 0 or 1,
R^{ck}, R^{cl}, and R^{cm} are each independently C₁₋₄ alkoxy;
R^{cn} is hydrogen or C₁₋₄ alkylcarbonyl;
R^{co} is C₁₋₂₀ alkyl optionally substituted by 1-3 groups R^{cq}, C₁₋₂₀ haloalkyl optionally substituted by 1-3 groups R^{cq}, or C₃₋₆ cycloalkyl;
each R^{cq} is independently: C₃₋₆ cycloalkyl, C₁₋₆ alkoxy, phenyl optionally substituted with 1-4 groups R^{cr}, or heteroaryl optionally substituted with 1-3 groups R^{cs};
and,
each R^{cr} and R^{cs} are independently: halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, C₁₋₄ alkoxycarbonyl, or amino.

3. The method according to claim 1 or claim 2, wherein:
A is a ring system selected from:
thiophene, furan, pyrrole, isoxazole, isothiazole, oxazole, thiazole, imidazole, pyrazole, 1,2,4-oxadiazole, 1,3,4-oxadiazole, 1,2,4-thiadiazole, 1,3,4-thiadiazole, 1,2,3-triazole, 1,2,4-triazole, pyridine, pyridazine, pyrimidine, pyrazine, 1,2,4-triazine, and 1,3,5-triazine, wherein said ring system is optionally substituted by 1-3 groups R¹;
each R¹ is independently: halogen, cyano, C₁₋₄ alkyl, C₁₋₄ haloalkyl, C₁₋₄ alkoxy, C₁₋₄ haloalkoxy, amino, C₁₋₄alkylamino, di(C₁₋₂alkyl)amino, C₁₋₄ alkylthio, or C₁₋₄ haloalkylthio; or 2 adjacent groups R¹ together with the atoms to which they are joined form a 6 membered aromatic ring, optionally substituted with 1-2 groups selected from halogen, cyano, C₁₋₄ alkyl, C₁₋₄ haloalkyl, C₁₋₄ alkoxy, C₁₋₄ haloalkoxy, C₁₋₄ alkylthio, and C₁₋₄ haloalkylthio;
X is NR⁵R⁶, wherein
R⁵ is: hydrogen; C₁₋₆alkyl optionally substituted by 1-4 groups R^{p}; C₁₋₆ haloalkyl optionally substituted by 1-4 groups R^{p}; SO₂R^{ss}; or C(O)R^{uu};
R⁶ is: hydrogen; C₁₋₆ alkyl optionally substituted by 1-4 groups R^{p}; or C₁₋₆ haloalkyl optionally substituted by 1-4 groups R^{p}; or R⁵ and R⁶ together with the N atom to which they are joined form a 3-8 membered ring system, said ring system optionally containing 1 or 2 further heteroatoms selected from O, N and S and being optionally substituted by 1-2 groups R^{v}; or R⁵ and R⁶ together form a group ═C(Rⁱ)OR^{j}, ═C(R^{k})SR^{L}, or ═C(R^{m})NRⁿR^{o};
Rⁱ is: hydrogen; C₁₋₄ alkyl optionally substituted by 1-4 groups R^{xx}; C₁₋₄ haloalkyl optionally substituted by 1-4 groups R^{xx}; C₁₋₄ alkoxy, C₁₋₄ alkylthio; or NR^{ac}R^{ad};
R^{j} is C₁₋₄ alkyl, or C₁₋₄ haloalkyl;
R^{k} is: hydrogen; C₁₋₄ alkyl optionally substituted by 1-4 groups R^{xx}; C₁₋₄ haloalkyl optionally substituted by 1-4 groups R^{xx}; C₁₋₄ alkylthio; or NR^{ac}R^{ad};
R^{L} is C₁₋₄ alkyl, or C₁₋₄ haloalkyl;
R^{m} is: hydrogen; C₁₋₄ alkyl optionally substituted by 1-4 groups R^{xx}; C₁₋₄ haloalkyl optionally substituted by 1-4 groups R^{xx}; C₃₋₆ cycloalkyl optionally substituted by 1-4 groups R^{yy}; phenyl optionally substituted by 1-3 groups R^{zz}; or NR^{ac}R^{ad};
Rⁿ is hydrogen, C₁₋₄ alkyl, or C₁₋₄ haloalkyl;
R^{o} is hydrogen, C₁₋₄ alkyl, or C₁₋₄ haloalkyl;
R^{p} is halogen, cyano, hydroxy, C₁₋₄ alkoxy, or C₂₋₈alkoxyalkoxy;
each R^{v} is independently: halogen; cyano; hydroxy; C₁₋₄ alkyl; C₁₋₄ haloalkyl; C₂₋₈alkoxyalkyl; C₂₋₄ alkenyl optionally substituted by one or more R^{ar}; C₁₋₄ alkoxy; C₁₋₄ haloalkoxy; C₁₋₄ alkylsulphonyl; C₁₋₄ haloalkylsulphonyl; or C(O)R^{an}; or any two geminal groups R^{v} together form an oxo group;
R^{ss} is C₁₋₄ alkyl, phenyl optionally substituted by 1-3 groups R^{zz}, or C₁₋₄ haloalkyl;
R^{uu} is: hydrogen; C₁₋₄ alkyl optionally substituted by 1-4 groups R^{xx}; C₁₋₄ haloalkyl optionally substituted by 1-4 groups R^{xx}; C₃₋₆ cycloalkyl optionally substituted by 1-4 groups R^{yy}; phenyl optionally substituted by 1-3 groups R^{zz}; heteroaryl optionally substituted by 1-3 groups R^{ab}; NR^{ac}R^{ad}; or OR^{ae};
each R^{xx} is independently cyano, C₁₋₄ alkoxy, C₃₋₆ cycloalkyl, or C₁₋₄ alkoxycarbonyl;
each R^{yy} is independently halogen, cyano or C₁₋₄ alkyl;
each R^{zz} and each R^{ab} is independently halogen, cyano, nitro, C₁₋₄alkyl, C₁₋₄ haloalkyl, C₂₋₈alkoxyalkyl, C₁₋₄ alkoxy, C₁₋₄haloalkoxy, C₁₋₄alkylsulphonyl, C₁₋₄haloalkylsulphonyl, or C(O)R^{an};
R^{ac} is hydrogen, C₁₋₄ alkyl, C₁₋₄ haloalkyl, or C₂₋₈alkoxyalkyl;
R^{ad} is hydrogen, C₁₋₄ alkyl, C₁₋₄ haloalkyl, or C₂₋₈alkoxyalkyl;
R^{ae} is: C₁₋₄ alkyl; phenylC₁₋₄alkyl optionally substituted by 1-3 groups R^{zz}; or phenyl optionally substituted by 1-3 groups R^{zz};
R^{an} is C₁₋₄ alkyl, C₁₋₄ haloalkyl, C₁₋₄alkoxy, C₁₋₄haloalkoxy, amino, C₁₋₄ alkylamino, or C₂₋₈dialkylamino;
each R^{ar} is independently halogen, cyano, nitro, or C₁₋₄ alkoxycarbonyl;
Y is: halogen, C₁₋₃ alkyl, C₁₋₃ haloalkyl, C₂₋₅ alkoxyalkyl, cyclopropyl optionally substituted by 1-2 groups R^{bc}, C₂₋₄ alkenyl, C₂₋₄ haloalkenyl, C₂₋₄ alkynyl optionally substituted by 1-2 groups R^{be};
wherein each R^{bc} is independently halogen or C₁₋₂ alkyl, and each R^{be} is independently halogen or C₃₋₉ trialkylsilyl;
Z is: (O)ₘ—(CR^{w}R^{x})ₙ—C(O)R^{cb}, wherein:
m is an integer of 0 or 1, n is an integer of 0 or 1, and n≧m;
R^{w} is hydrogen or C₁₋₄ alkyl and R^{x} is hydrogen;
R^{cb} is hydroxy, C₁₋₁₀ alkylthio, or OR^{co};
R^{co} is C₁₋₂₀ alkyl optionally substituted by 1-2 groups R^{cq} or C₁₋₂₀ haloalkyl optionally substituted by 1-2 groups R^{cq};
each R^{cq} is independently phenyl optionally substituted with 1-3 groups R^{cr}, or heteroaryl optionally substituted with 1-2 groups R^{cs};
each R^{cr} is independently halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, or C₁₋₄ alkoxycarbonyl;
and,
each R^{cs} is independently halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, C₁₋₄ or alkoxycarbonyl.

4. The method according to any one of claims 1 to 3, wherein:
A is a ring selected from: thiophene, thiazole, 1,2,3-triazole, and pyridine, wherein said ring is optionally substituted by 1-3 groups R¹;
each R¹ is independently selected from: halogen, cyano, C₁₋₂ alkyl, C₁₋₂ haloalkyl, C₁₋₂alkoxy, C₁₋₂ haloalkoxy, and di(C₁₋₂alkyl)amino; or 2 adjacent groups R¹ together with the atoms to which they are joined form a 6 membered aromatic ring optionally substituted with 1-2 groups independently selected from: halogen, cyano, C₁₋₂ alkyl, C₁₋₂ haloalkyl, C₁₋₂alkoxy, and C₁₋₂haloalkoxy;
X is NR⁵R⁶, wherein
R⁵ is: hydrogen; C₁₋₄ alkyl optionally substituted by 1-2 groups R^{p}; C₁₋₄ haloalkyl optionally substituted by 1-2 groups R^{p}; SO₂R^{ss}; or C(O)R^{uu};
R⁶ is: hydrogen; C₁₋₄ alkyl optionally substituted by 1-2 groups R^{p}; or C₁₋₄ haloalkyl optionally substituted by 1-2 groups R^{p}; or R⁵ and R⁶ together with the N atom to which they are joined form a 4-6 membered ring system, said ring system optionally containing 1 further heteroatom selected from O, N, and S and being optionally substituted by 1-2 groups R^{v}; or R⁵ and R⁶ together form a group selected from: ═C(Rⁱ)OR^{j}, ═C(R^{k})SR^{L}, and ═C(R^{m})NRⁿR^{o};
Rⁱ is hydrogen, C₁₋₄ alkyl, C₁₋₄ alkoxy, C₁₋₄ alkylthio, or NR^{ac}R^{ad};
R^{j} is C₁₋₄ alkyl;
R^{k} is hydrogen, C₁₋₄ alkyl, C₁₋₄ alkylthio, or NR^{ac}R^{ad};
R^{L} is C₁₋₄ alkyl;
R^{m} is hydrogen, C₁₋₄ alkyl, Cₘcycloalkyl, phenyl, or NR^{ac}R^{ad};
Rⁿ and R^{o} are each independently hydrogen, or C₁₋₄ alkyl;
each R^{p} is independently halogen, hydroxy, or C₁₋₄ alkoxy;
each R^{v} is independently halogen, or C₁₋₄ alkyl;
R^{ss} is C₁₋₄alkyl, or phenyl optionally substituted with 1-3 groups R^{zz};
R^{uu} is: C₁₋₄alkyl; phenyl optionally substituted with 1-3 groups R^{zz}; C₁₋₄ alkoxy; or NR^{ac}R^{ad};
R^{zz} is halogen, C₁₋₄alkyl, C₁₋₄ alkoxy, or C₁₋₄ alkylsulphonyl;
R^{ac} and R^{ad} are each independently hydrogen or C₁₋₄ alkyl;
Y is: halogen, C₁₋₂ alkyl, C₁₋₂ haloalkyl, C₂₋₃ alkoxyalkyl, or C₂₋₃ alkenyl;
Z is C(O)R^{cb} wherein R^{cb} is hydroxy, C₁₋₁₀ alkoxy, or phenyl(C₁₋₂)alkoxy.

5. A compound of formula (IA) 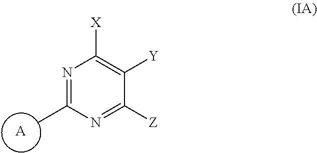
wherein A, X, and Z are independently as defined in any one of claims 1 to 4, Y is NR⁵R⁶ or halogen, and R⁵ and R⁶ are as defined in claim 1.

6. A herbicidal composition comprising a compound of formula (IA) as defined in claim 5 and at least one agriculturally acceptable formulation adjuvant or diluent.

7. The herbicidal composition according to claim 6, further comprising a crop safener.

8. A compound of formula (I) as defined in any one of claims 1 to 4, or a compound of formula (IA) as defined in claim 5, or a herbicidal composition according to claim 6 or claim 7, in admixture with at least one active ingredient selected from the group consisting of: an insecticide, an acaricide, a nematocide, a molluscicide, an herbicide, a fungicide, and a plant growth regulator.

9. Use of a compound of formula (I) as defined in any one of claims 1 to 4, or a compound of formula (IA) as defined in claim 5, as an herbicide.

10. A method of controlling undesired plant growth, which comprises applying to said undesired plants a compound of formula (IA) as defined in claim 5, or an herbicidal composition as defined in claim 6 or claim 7, or mixture according to claim 8.

11. A method of controlling weeds in crops of useful plants which comprises applying to said weeds or to the locus of said weeds, or to said crop of useful plants, a compound of formula (I) as defined in any one of claims 1 to 4, a compound of formula (IA) as defined in claim 5, an herbicidal composition according to claim 6 or claim 7, or a mixture according to claim 8.
